(19) United States
(12) Patent Application Publication
(10) Pub. No.: US 2005/0096331 A1
(43) Pub. Date: May 5, 2005

(54) Novel compounds and their use in medicine process for their preparation and pharmaceutical compositions containing them

(76) Inventors: Saibal Kumar Das, Hyderabad, Andhra Pradesh (IN); Debnath Bhuniya, Hyderabad, Andhra Pradesh (IN); Gurram, Ranga Madhavan, Hyderabad, Andhra Pradesh (IN); Javed Iqbal, Hyderabad, Andhra Pradesh (IN); Ranjan Chakrabarti, Hyderabad, Andhra Pradesh (IN)

Correspondence Address: LADAS & PARRY, 26 WEST 61ST STREET, NEW YORK, NY, 10023, US

(21) Appl. No.: 10/499,852

(22) PCT Filed: Dec. 17, 2002
(86) PCT No.: PCT/IB02/05442

(30) Foreign Application Priority Data
Dec. 21, 2001 (IN) 1029/MAS/2001

(51) Int. Cl. A61K 31/519; C07D 487/04

(52) U.S. Cl. 514/259.3; 514/259.31; 544/280; 544/281

(57) ABSTRACT

The present invention relates to novel antidiabetic, hypolipidemic, antiobesity and hypocholesterolemic compounds, their derivatives, their analogs, their tautomeric forms, their stereoisomers, their polymorphs, their pharmaceutically acceptable salts, their pharmaceutically acceptable solvates and pharmaceutically acceptable compositions containing them. More particularly, the present invention relates to novel alkyl carboxylic acids of the general formula (I), their derivatives, their analogs, their tautomeric forms, their stereoisomers, their polymorphs, their pharmaceutically acceptable salts, their pharmaceutically acceptable solvates and pharmaceutically acceptable compositions containing them.

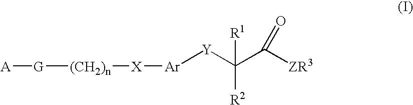

### FIELD OF THE INVENTION

The present invention relates to novel antidiabetic, hypolipidemic, antiobesity and hypocholesterolemic compounds, their derivatives, their analogs, their tautomeric forms, their stereoisomers, their polymorphs, their pharmaceutically acceptable salts, their pharmaceutically acceptable solvates and pharmaceutically acceptable compositions containing them. More particularly, the present invention relates to novel alkyl carboxylic acids of the general formula (I), their derivatives, their analogs, their tautomeric forms, their stereoisomers, their polymorphs, their pharmaceutically acceptable salts, their pharmaceutically acceptable solvates and pharmaceutically acceptable compositions containing them.

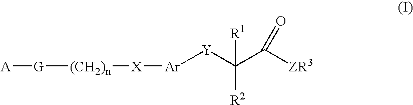

The present invention also relates to a process for the preparation of the above said compounds, their analogs, their derivatives, their tautomeric forms, their stereoisomers, their polymorphs, their pharmaceutically acceptable salts, their pharmaceutically acceptable solvates and pharmaceutical compositions containing them.

The present invention also relates to novel intermediates, processes for their preparation, their use in the preparation of compounds of formula (I) and their use as antidiabetic, hypolipidemic, antiobesity and hypocholesterolemic compounds.

The present invention also relates to novel intermediates, processes for their preparation and their use in the preparation of compounds of formula (I).

The compounds of the present invention lower plasma glucose, triglycerides, lower total cholesterol (TC) and increase high density lipoprotein (HDL) and decrease low density lipoprotein (LDL), which have a beneficial effect on coronary heart disease and atherosclerosis.

The compounds of general formula (I) are useful in reducing body weight and for the treatment and/or prophylaxis of diseases such as atherosclerosis, stroke, peripheral vascular diseases and related disorders. These compounds are useful for the treatment of hyperlipidemia, hyperglycemia, hypercholesterolemia, lowering of atherogenic lipoproteins, VLDL (very low density lipoprotein) and LDL. The compounds of the present invention can be used for the treatment of certain renal diseases including glomerulonephritis, glomerulosclerosis, nephrotic syndrome, hypertensive nephrosclerosis and nephropathy. The compounds of general formula (I) are also useful for the treatment and/or prophylaxis of leptin resistance, impaired glucose tolerance, disorders related to syndrome X such as hypertension, obesity, insulin resistance, coronary heart disease and other cardiovascular disorders. These compounds may also be useful as aldose reductase inhibitors, for improving cognitive functions in dementia, treating diabetic complications, disorders related to endothelial cell activation, psoriasis, polycystic ovarian syndrome (PCOS), inflammatory bowel diseases, osteoporosis, myotonic dystrophy, pancreatitis, arteriosclerosis, retinopathy, xanthoma, eating disorders, inflammation and for the treatment of cancer. The compounds of the present invention are also useful in the treatment and/or prophylaxis of the above said diseases in combination/concomittant with one or more HMG CoA reductase inhibitor; cholesterol absorption inhibitor; antiobesity drug; lipoprotein disorder treatment drug; hypoglycemic agent; insulin; biguanide; sulfonylurea; thiazolidinedione; dual PPARα and γ agonists or a mixture thereof.

### BACKGROUND OF THE INVENTION

Atherosclerosis and other peripheral vascular diseases effect the quality of life of millions of people. Therefore, considerable attention has been directed towards understanding the etiology of hypercholesterolemia and hyperlipidemia and development of effective therapeutic strategies.

Hypercholesterolemia has been defined as plasma cholesterol level that exceeds arbitrarily defined value called “normal” level. Recently, it has been accepted that “ideal” plasma levels of cholesterol are much below the “normal” level of cholesterol in the general population and the risk of coronary artery disease (CAD) increases as cholesterol level rises above the “optimum” (or “ideal”) value. There is clearly a definite cause and effect-relationship between hypercholesterolemia and CAD, particularly for individuals with multiple risk factors. Most of the cholesterol is present in the esterified forms with various lipoproteins such as Low density lipoprotein (LDL), Intermediate density lipoprotein (IDL), High density lipoprotein (HDL) and partially as Very low density lipoprotein (VLDL). Studies clearly indicate that there is an inverse correlationship between CAD and atherosclerosis with serum HDL-cholesterol concentrations, (Stampfer et al., N. Engl. J. Med., 325 (1991), 373-381) and the risk of CAD increases with increasing levels of LDL and VLDL.

In CAD, generally “fatty streaks” in carotid, coronary and cerebral arteries, are found which are primarily free and esterified cholesterol. Miller et al, (Br. Med. J, 282 (1981), 1741-1744) have shown that increase in HDL-particles may decrease the number of sites of stenosis in coronary arteries of human, and high level of HDL-cholesterol may protect against the progression of atherosclerosis. Picardo et al, Arteriosclerosis 6 (1986) 434-441 have shown by in vitro experiment that HDL is capable of removing cholesterol from cells. They suggest that HDL may deplete tissues of excess free cholesterol and transfer it to liver, which is known as reverse cholesterol transport, (Macikinnon et al., J. Biol. chem. 261 (1986), 2548-2552). Therefore, agents that increase HDL cholesterol would have therapeutic significance for the treatment of hypercholesterolemia and coronary heart diseases (CHD).

Obesity is a disease highly prevalent in affluent societies and in the developing world and is a major cause of morbidity and mortality. It is a state of excess body fat accumulation. The causes of obesity are unclear. It is believed to be of genetic origin or promoted by an interaction between the genotype and environment. Irrespective of the cause, the result is fat deposition due to imbalance between the energy intake versus energy expenditure. Dieting, exercise and appetite suppression have been a part of obesity treatment. There is a need for efficient therapy to fight this disease since it may lead to coronary heart disease, diabetes, stroke, hyperlipidemia, gout, osteoarthritis, reduced fertility and many other psychological and social problems.

Diabetes and insulin resistance is yet another disease which severely effects the quality of large population in the world. Insulin resistance is the diminished ability of insulin to exert its biological action across a broad range of concentrations. In insulin resistance, the body secretes abnormally high amounts of insulin to compensate for this defect; failing which, the plasma glucose concentration inevitably raises and develops into diabetes. Among the developed countries, diabetes mellitus is a common problem and is associated with a variety of abnormalities including obesity, hypertension, hyperlipidemia (J. Clin. Invest., 75 (1985) 809-817; N. Engl. J. Med 317 (1987) 350-357; J. Clin. Endocrinol. Metab., 66 (1988) 580-583; J. Clin. Invest., 68 (1975) 957-969) and other renal complications (patent publication No. WO 95/21608). It is now increasingly being recognized that insulin resistance and relative hyperinsulinemia have a contributory role in obesity, hypertension, atherosclerosis and type 2 diabetes mellitus. The association of insulin resistance with obesity, hypertension and angina has been described as a syndrome having insulin resistance as the central pathogenic link-Syndrome-X.

Hyperlipidemia is the primary cause for cardiovascular (CVD) and other peripheral vascular diseases. High risk of CVD is related to the higher LDL (Low Density Lipoprotein) and VLDL (Very Low Density Lipoprotein) seen in hyperlipidemia. Patients having glucose intolerance/insulin resistance in addition to hyperlipidemia have higher risk of CVD. Numerous studies in the past have shown that lowering of plasma triglycerides and total cholesterol, in particular LDL and VLDL and increasing HDL cholesterol help in preventing cardiovascular diseases.

Peroxisome proliferator activated receptors (PPAR) are members of the nuclear receptor super family. The gamma (γ) isoform of PPAR (PPARγ) has been implicated in regulating differentiation of adipocytes (Endocrinology, 135 (1994) 798-800) and energy homeostasis (Cell, 83 (1995) 803-812), whereas the alpha (α) isoform of PPAR (PPARα) mediates fatty acid oxidation (Trend Endocrin. Metab., 4 (1993) 291-296) thereby resulting in reduction of circulating free fatty acid in plasma (Current Biol. 5 (1995) 618-621). PPARα agonists have been found useful for the treatment of obesity (WO 97/36579). It has been recently disclosed that compounds which are agonists for both PPARα and PPARγ are suggested to be useful for the treatment of syndrome X (WO 97/25042). Similar effect between the insulin sensitizer (PPARγ agonist) and HMG CoA reductase inhibitor has been observed which may be useful for the treatment of atherosclerosis and xanthoma (EP 0 753 298).

It is known that PPARγ plays an important role in adipocyte differentiation (Cell, 87 (1996) 377-389). Ligand activation of PPAR is sufficient to cause complete terminal differentiation (Cell, 79 (1994) 1147-1156) including cell cycle withdrawal. PPARγ is consistently expressed in certain cells and activation of this nuclear receptor with PPARγ agonists would stimulate the terminal differentiation of adipocyte precursors and cause morphological and molecular changes characteristics of a more differentiated, less malignant state (Molecular Cell, (1998), 465-470; Carcinogenesis, (1998), 1949-53; Proc. Natl. Acad. Sci., 94 (1997) 237-241) and inhibition of expression of prostate cancer tissue (Cancer Research 58 (1998) 3344-3352). This would be useful in the treatment of certain types of cancer, which express PPARγ and could lead to a quite nontoxic chemotherapy.

Leptin resistance is a condition wherein the target cells are unable to respond to leptin signal. This may give rise to obesity due to excess food intake and reduced energy expenditure and cause impaired glucose tolerance, type 2 diabetes, cardiovascular diseases nd such other interrelated complications. Kallen et al (Proc. Natl. Acad. Sci. (1996) 93, 5793-5796) have reported that insulin sensitizers which perhaps due to the PPAR agonist expression lower plasma leptin concentrations. However, it has been recently disclosed that compounds having insulin sensitizing property also possess leptin sensitization activity. They lower the circulating plasma leptin concentrations by improving the target cell response to leptin (WO 98/02159).

A few alkyl carboxylic acids, their derivatives and their analogs have been reported to be useful in the treatment of hyperglycemia and hypercholesterolemia. Some of such compounds described in the prior art are outlined below:
i). In our international publication No. WO 99/08501 we have disclosed the compounds of general formula (IIa) 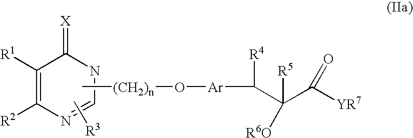
   where X represents O or S; the groups R¹, R² and group R³ when attached to the carbon atom, may be same or different and represent hydrogen, halogen, hydroxy, nitro, cyano, formyl or optionally substituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, acyl, acyloxy, hydroxyalkyl, amino, acylamino, alkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R¹, R² along with the adjacent atoms to which they are attached may also form a 5-6 membered substituted or unsubstituted cyclic structure containing carbon atoms with one or more double bonds, which may optionally contain one or more heteroatoms selected from oxygen, nitrogen and sulfur; R³ when attached to nitrogen atom represents hydrogen, hydroxy, formyl or optionally substituted groups selected from alkyl, cycloalkyl, alkoxy, cycloalkoxy, aryl, aralkyl, heterocyclyl, heteroaryl, heteroaralkyl, acyl acyloxy, hydroxyalkyl, amino, acylamino, alkylamino, arylamino, aralkylamino, aminoalkyl, aryloxy, aralkoxy, heteroaryloxy, heteroaralkoxy, alkoxycarbonyl, aryloxycarbonyl, aralkoxycarbonyl, alkoxyalkyl, aryloxyalkyl, aralkoxyalkyl, alkylthio, thioalkyl groups, carboxylic acid derivatives, or sulfonic acid derivatives; the linking group represented by —(CH₂)ₙ—O— may be attached either through nitrogen atom or through carbon atom where n is an integer ranging from 1-4; Ar represents an optionally substituted divalent single or fused aromatic or heterocyclic group; R⁴ represents hydrogen atom, hydroxy, alkoxy, halogen, lower alkyl, optionally substituted aralkyl group or forms a bond together with the adjacent group R⁵; R⁵ represents hydrogen, hydroxy, alkoxy, halogen, lower alkyl group, acyl, optionally substituted aralkyl or R⁵ forms a bond together with R⁴; R⁶ may be hydrogen, optionally substituted groups selected from alkyl cycloalkyl, aryl, aralkyl, alkoxyalkyl, alkoxycarbonyl, aryloxycarbonyl, alkylaminocarbonyl, arylaminocarbonyl, acyl, heterocyclyl, heteroaryl, heteroaralkyl groups, with a provision that R⁶ does not represent hydrogen when R⁷ represents hydrogen or lower alkyl group; R⁷ may be hydrogen or optionally substituted groups selected from alkyl, cycloalkyl, aryl, aralkyl, heterocyclyl, heteroaryl, heteroaralkyl groups; Y represents oxygen or NR⁸, where R⁸ represents hydrogen, alkyl, aryl, hydroxyalkyl, aralkyl, heterocyclyl, heteroaryl, heteroaralkyl groups; R⁷ and R⁸ together may form a substituted or unsubstituted 5 or 6 membered cyclic structure containing carbon atoms, which may optionally contain one or more heteroatoms selected from oxygen, sulfur or nitrogen.

An example of these compounds is shown in formula (IIj) 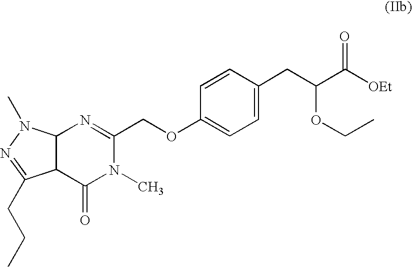
ii) International publication No. WO 00/64888 disclose the compounds of general formula (IIc) 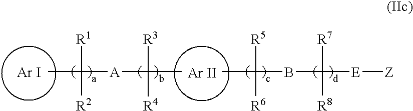
   wherein Ar¹ and Ar² are independently aryl, fused arylcycloalkenyl, fused arylcycloalkyl, fused arylheterocyclenyl, fused arylheterocyclyl, heteroaryl, fused heteroarylcycloalkenyl, fused heteroarylcycloalkyl, fused heteroarylcyclenyl or fused heteroarylheterocyclyl; A is O, S, SO, SO₂, NR¹³, C(O), NR¹⁴C(O), C(O)NR¹⁴, NR¹⁴C(O)N(R¹⁵), C(R¹⁴)═N; chem bond and the like; B is O, S, NR¹⁹, a chemical bond, C(O), N(R²⁰)C(O) or C(O)N(R²⁰); E is a chemical bond or an ethylene group; a is 0-6; b is 0-4; c is 0-4; d is 0-6; g is 1-5; h is 1-4; R¹, R³, R⁵ and R⁷ are independently hydrogen, halogen, alkyl, carbonyl, alkoxycarbonyl, or aralkyl; R², R⁴, R⁶ and R⁸ are independently —(CH₂)_{q}—X; q is 0-3; X is hydrogen, halogen, alkyl, alkenyl, cycloalkyl, heterocyclyl, aryl, heteroaryl, aralkyl, heteroaralkyl, hydroxy, alkoxy, aralkoxy, heteroaralkoxy, carbonyl, alkoxycarbonyl, tetrazolyl, acyl, acylHNSO₂, and the like; Z is R²¹O₂C, R²¹OC, cyclo-imide; CN, R²¹O₂SHNCO, R²¹O₂SNH, R²¹NCO, R²¹O-2,4-thiazolidinonyl or tetrazolyl.

An example of these compounds is shown in formula (IId) 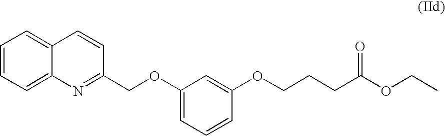
iii) International publication Nos. WO 95/03038 and WO 96/04260 disclose compounds of formula (II e) 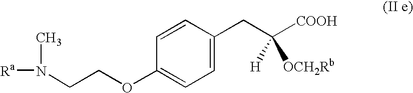
   wherein R^{a} represents 2-benzoxazolyl or 2-pyridyl and R^{b} represent CF₃, CH₂OCH₃ or CH₃. A typical example is (S)-3-[4-[2-[N-2-benzoxazolyl)N-methylamino]ethoxy]phenyl]-2-(2,2,2-trifluoroethoxy)propanoic acid (II f). 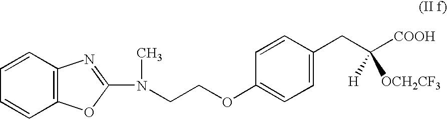
iv) International publication Nos. WO 94/13650, WO 94/01420 and WO 95/17394 disclose the compounds of general formula (II g)
   A¹-X—(CH₂)ₙ—O-A²-A³-Y.R² (II g)
   wherein A¹ represent aromatic heterocycle, A² represents substituted benzene ring and A³ represents moiety of formula (CH₂)ₘ—CH—(OR¹), wherein R¹ represents alkyl groups, m is an integer of 1-5; X represents substituted or unsubstituted N; Y represents C═O or C═S, R² represents OR³ where R³ may be hydrogen, allyl, aralkyl, or aryl group and n is an integer of 2-6.

An example of these compounds is shown in formula (II h) 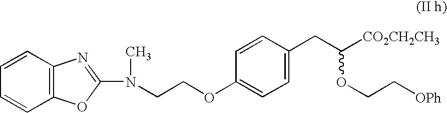
v) International publication No. WO 00/49005 disclose the compounds of general formula (II i) 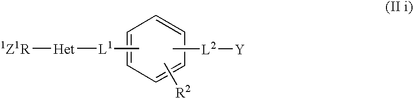
   wherein Het is an optionally substituted, saturated partially saturated or fully unsaturated 8 to 10 membered bicyclic ring, R¹ is optionally substituted aryl or optionally substituted heteroaryl, R² is hydrogen halogen, lower alkyl or lower alkoxy, L¹ is an —R³-R⁴ linkage where R³ is alkylene, alkenylene or alkynylene and R⁴ is a direct bond, cycloalkylene, heterocycloalkylene, arylene, heteroarylidinyl, —C(=Z²)-NR⁵, NR⁵—C(=Z²), -Z²-, —C(═O), —C(═NOR⁵)—, —NR⁵—, NR⁵—C(=Z²)—NR⁵, SO₂—NR⁵NR⁵—SO₂, —O—C(═O)—, —C(═O)—O, —O—C(═O)—NR⁵, —NR⁵—C(═O)—O—; L² is optionally substituted alkylene or alkenylene, Y is carboxy or an acid bioisostere and Z¹ is NR⁵ and the corresponding N-oxides and their prodrugs and pharmaceutically acceptable salts and solvates.

An example of these compounds is shown in formula (II j) 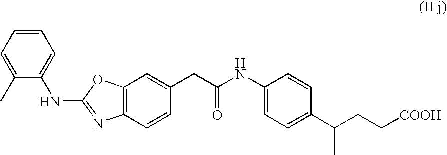
vi) International publication No. WO 94/12181 disclose the compounds of general formula (II k)
   X—Y-Z-Aryl-A-B (II k)
   aryl is a 6 membered aromatic ring containing 0, 1, 2 or 3 nitrogen atoms and either unsubstituted or substituted with R⁸ and R⁹; X represents NH₂, NH—C(═NH)—, and the like or 4 to 10 membered mono or polycyclic aromatic or nonaromatic ring system and containing 0, 1, 2, 3 or 4 heteroatoms selected from N, O or S either unsubstituted or substituted; Y is selected from C₀₋₈ alkyl, C₄₋₁₀ cycloalkyl, C₀₋₈ alkyl-NR³—C₀₋₈ alkyl, C₀₋₈alkyl-CONR³—C₀₋₈ alkyl, C₀₋₈ alkyl-O—C₀₋₈ alkyl, C₀₋₈ alkyl-S(O)ₙ—C₀₋₈ alkyl, (CH₂)₀₋₈ aryl-(CH₂)₀₋₈, (CH₂)₀₋₆ aryl-SOₙ—, (CH₂)₀₋₈ aryl-CO—(CH₂)₀₋₈, (CH₂)₀₋₆ aryl-SO₂—(CH₂)₀₋₆, (CH₂)₀₋₆ NR³—(CH₂)₀₋₆, (CH₂)₀₋₆ aryl CH(OH)—(CH₂)₀₋₆—, (CH₂)₀₋₈—CONH—(CH₂)₀₋₈—, C₀₋₈ alkyl-SO₂—NR³—C₀₋₈ alkyl, C₀₋₈ alkyl-CO—C₀₋₈ alkyl, C₀₋₈ alkyl-CH(OH)—C₀₋₈ alkyl, where n is an integer from 0-2; Z and A are independently chosen from (CH₂)ₘ, (CH₂)ₘO(CH₂)ₙ, (CH₂)ₘNR³(CH₂)ₙ, (CH₂)ₘNR³(CH₂)ₙ, (CH₂)ₘCONR¹¹(CH₂)ₙ, (CH₂)ₘCO(CH₂)ₙ, (CH₂)ₘCS(CH₂)ₙ, (CH₂)ₘSO₂(CH₂)ₙ, (CH₂)ₘS(CH₂)ₙ, (CH₂)ₘSO₂(CH₂)ₙ, (CH₂)ₘSO(CH₂)ₙ, (CH₂)ₘSO₂NR³(CH₂)ₙ, (CH₂)ₘNR³SO₂(CH₂)ₘCR³═CR⁴(CH₂)ₙ, (CH₂)ₘC≡C(CH₂)ₙ, (CH₂)ₘCH(OH)(CH₂)ₙ; where m and n are each independently an integer from 0 to 6; Aryl is a 6 membered aromatic ring system containing 0, 1, 2, 3 or 4 N atoms and either unsubstituted or substituted with R⁵, provided that when A is (CH₂)ₙ, the Aryl ring, bonded by Z and A must contain at least one heteroatom;
B is 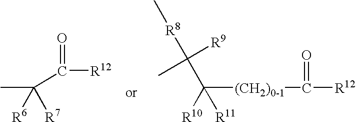
R⁶, R⁷, R⁸, R⁹, R¹⁰ and R¹¹, are independently selected from hydrogen, fluorine, (C₁₋₈) alkyl, hydroxy, hydroxy(C₁₋₆)alkyl, carboxy(C₀₋₆)alkyl, (C₁₋₆)alkyloxy, aryl(C₀₋₆)alkyloxy, (C₃₋₈)cycloalkyl, aryl(C₀₋₆)alkyl, (C₁₋₆)alkylcarbonyloxy, (C₀₋₆)alkylamino(C₀₋₆)alkyl and the like; R¹² is selected from hydroxy, (C₁₋₈)alkyloxy, aryl(C₀₋₆)alkyl and the like;

An example of these compounds is shown in formula (II l) 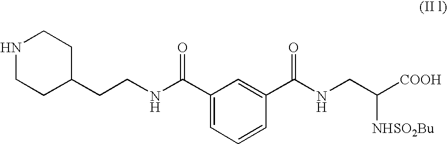
vii) International publication No. WO 93/16697 and U.S. Pat. No. 5,227,490 disclose the compounds of general formula (II m) 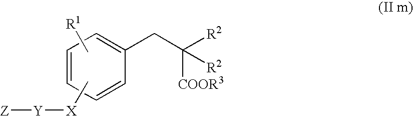
   R¹ is chosen from hydrogen, C₁₋₆ alkyl, aryl C₄₋₁₀ alkyl, aryl, carboxy, C₁₋₆ alkyloxy, carboxy C₀₋₆ alkyl, carboxy C₁₋₆ alkyloxy, hydroxy C₀₋₆ alkyl, C₁₋₄ alkylsulfonyl C₀₋₆ alkyl, C₀₋₄ alkylamino C₀₋₆ alkyl, aryl C₀₋₁₀ alkylamino C₀₋₆ alkyl, C₂₋₁₀ acylamino C₀₋₆ alkyl, C₁₋₄ carboalkoxy C₀₋₆ alkyl halogen, R² is independently chosen from hydrogen, halogen, hydroxy, C₁₋₆ alkyl, wherein the alkyl group is substituted or unsubstituted, C₁₋₆alkyloxy, aryl C₀₋₄ alkyl, aryl C₀₋₆ alkyloxy and the like; R³ hydrogen, C₀₋₆ alkyl, aryl C₁₋₁₀ alkyl; Z is NR⁴R⁵ or a 4-9 membered mono or bicyclic ring system containing 1, 2 or 3 heteroatoms selected from N, O or S and either unsubstituted or substituted; Y is C₁₋₆ alkyl either unsubstituted or substituted, C₄₋₈ cycloalkyl, aryl, —C(═O)NH—, —NH(C═O)— and the like; X is O, SO, SO₂, S, CO, —NReCO—, CONR⁴—, CH₂ and the like;

An example of these compounds is shown in formula (II n)

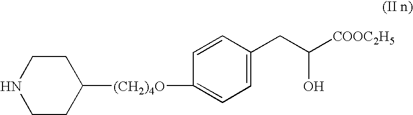

### SUMMARY OF THE INVENTION

With an objective to develop novel compounds for reducing blood glucose, lipid levels, lowering cholesterol and reducing body weight with beneficial effects in the treatment and/or prophylaxis of diseases related to increased levels of lipids, atherosclerosis, coronary artery diseases, Syndrome-X, impaired glucose tolerance, insulin resistance, insulin resistance leading to type 2 diabetes and diabetic complications thereof, for the treatment of diseases wherein insulin resistance is the pathophysiological mechanism and for the treatment of hypertension, with better efficacy, potency and lower toxicity, we focused our research to develop new compounds effective in the treatment of the above mentioned diseases. Effort in this direction has led to compounds having general formula (I).

The main objective of the present invention is therefore, to provide novel alkyl carboxylic acids, their derivatives, their analogs, their tautomeric forms, their stereoisomers, their polymorphs, their pharmaceutically acceptable salts, their pharmaceutically acceptable solvates and pharmaceutical compositions containing them, or their mixtures.

Another objective of the present invention is to provide novel alkyl carboxylic acids, their derivatives, their analogs, their tautomeric forms, their stereoisomers, their polymorphs, their pharmaceutically acceptable salts, their pharmaceutically acceptable solvates and pharmaceutical compositions containing them or their mixtures which may have agonist activity against PPARα and/or PPARγ, and optionally inhibit HMG CoA reductase, in addition to having agonist activity against PPARα and/or PPARγ.

Another objective of the present invention is to provide novel alkyl carboxylic acids, their derivatives, their analogs, their tautomeric forms, their stereoisomers, their polymorphs, their pharmaceutically acceptable salts, their pharmaceutically acceptable solvates and pharmaceutical compositions containing them or their mixtures having enhanced activities, without toxic effect or with reduced toxic effect.

Yet another objective of the present invention is to provide a process for the preparation of alkyl carboxylic acids of formula (I), their derivatives, their analogs, their tautomeric forms, their stereoisomers, their polymorphs, their pharmaceutically acceptable salts and their pharmaceutically acceptable solvates.

Still another objective of the present invention is to provide pharmaceutical compositions containing compounds of the general formula (I), their analogs, their derivatives, their tautomers, their stereoisomers, their polymorphs, their salts, solvates or their mixtures in combination with suitable carriers, solvents, diluents and other media normally employed in preparing such compositions.

Another objective of the present invention is to provide novel intermediates, a process for their preparation and use of the intermediates in processes for preparation of alkyl carboxylic acids of formula (I), their derivatives, their analogs, their tautomers, their stereoisomers, their polymorphs, their salts and their pharmaceutically acceptable solvates and their use as antidiabetic, hypolipidemic, antiobesity and hypocholesterolemic compounds.

### DETAILED DESCRIPTION OF THE INVENTION

Novel alkyl carboxylic acids of compound of the general formula (I) 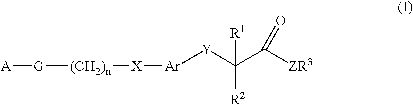
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, alkanoyl, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, eteroaryl or heteroaralkyl groups; Z represents oxygen or NR⁴, where R⁴ presents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, alkanoyloxy, aroyl, aralkanoyl, heterocyclyl, heteroaryl, heteroaralkyl groups or (C₁-C₁₂)alkylcarboxylic acid or its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 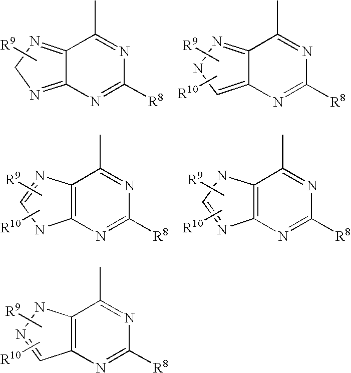
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups, selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives.

Suitable groups represented by R¹ may be selected from hydrogen, hydroxy, (C₁-C₆)alkyl groups such as methyl, ethyl, propyl and the like; (C₁-C₆)alkoxy such as methoxy, ethoxy, propoxy and the like; halogen atom such as fluorine, chlorine, bromine or iodine; (C₂-C₁₀)alkanoyl group such as acetyl, propanoyl, butanoyl, pentanoyl, benzoyl and the like; aralkyl such as benzyl, phenethyl and the like, which may be unsubstituted or substituted or R¹ together with R⁷ forms a bond. The substituents are selected from halogen, hydroxy or alkyl groups.

Suitable groups represented by R² may be selected from hydrogen, hydroxy, halogen atom such as chlorine, bromine, iodine or fluorine; linear or branched (C₁-C₁₆) alkyl, preferably (C₁-C₁₂)alkyl group such as methyl, ethyl, n-propyl, i-propyl, n-butyl, i-butyl, pentyl, hexyl, heptyl, octyl and the like, the alkyl group may be substituted; (C₁-C₁₀)alkoxy such as methoxy, ethoxy, propyloxy, butyloxy, iso-propyloxy, hexyloxy, octyloxy and the lice, which may be substituted; (C₃-C₇)cycloalkyl group such as cyclopropyl, cyclobutyl, cyclopentyl, cyclohexyl and the like, the cycloalkyl group may be substituted; (C₃-C₇)cycloalkyl(C₁-C₁₀)alkyl group such as cyclohexylmethyl, cyclohexylethyl, cyclohexylpropyl, cyclohexylbutyl and the like, which may be substituted; aryl group such as phenyl, naphthyl and the like, the aryl group may be substituted; aralkyl group such as benzyl, phenethyl and the like, the aralkyl group may be substituted; aryloxy group such as phenoxy, naphthyloxy and the like, the aryloxy group may be substituted; aralkoxy group such as benzyloxy, phenethyloxy, naphthylmethyloxy, phenylpropyloxy and the like, the aralkoxy group may be substituted; heteroaryl group such as pyridyl, thienyl, pyrrolyl, furyl and the like, the heteroaryl group may be substituted; heteroaralkyl group such as furanmethyl, pyridinemethyl, oxazolemethyl, oxazolethyl and the like, the heteroaralkyl group may be substituted; heteroaryloxy and heteroaralkoxy, wherein heteroaryl and heteroaralkyl moieties are as defined earlier and may be substituted; heterocyclyl group such as aziridinyl, pyrrolidinyl, piperidinyl and the like, the heterocyclyl group may be substituted; linear or branched (C₂-C₁₆)alkanoyl group such as acetyl, propanoyl, butanoyl, benzoyl, octanoyl, decanoyl and the like, which may be substituted; alkanoyloxy group such as OOCMe, OOCEt, OOCPh and the like which may be substituted; alkoxyalkyl group such as methoxymethyl, ethoxymethyl, methoxyethyl, ethoxyethyl and the like, the alkoxyalkyl group may be substituted; (C₁-C₆)alkoxycarbonyl group such as methoxycarbonyl, ethoxycarbonyl and the like, the alkoxycarbonyl group may be substituted; aryloxycarbonyl such as phenoxycarbonyl, naphthyloxycarbonyl and the like, the aryl group may be substituted; (C₁-C₆)alkylaminocarbonyl such as methylaminocarbonyl, ethylaminocarbonyl, propylaminocarbonyl and the like, which may be substituted; arylaminocarbonyl such as PhNHCO, naphthylaminocarbonyl and the like, the aryl moiety may be substituted. The substituents may be selected from halogen, hydroxy, nitro or unsubstituted or substituted groups selected from alkyl, cycloalkyl, alkoxy, cycloalkoxy, aryl, aralkyl, aralkoxyalkyl, heterocyclyl, heteroaryl, heteroaralkyl, alkanoyl, alkanoyloxy, hydroxyalkyl, amino, alkanoylamino, arylamino, aminoalkyl, aryloxy, aralkoxy, alkoxycarbonyl, alkylamino, alkoxyalkyl, aryloxyalkyl, alkylthio, thioalkyl groups, carboxylic acid or its derivatives or sulfonic acid or its derivatives.

Suitable groups represented by R³ may be selected from hydrogen, linear or branched (C₁-C₁₆)alkyl, preferably (C₁-C₁₂)alkyl group such as methyl, ethyl, n-propyl, i-propyl, n-butyl, i-butyl, pentyl, hexyl, heptyl, octyl and the like, the alkyl group may be substituted; (C₃-C₇)cycloalkyl such as cyclopropyl, cyclopentyl, cyclohexyl and the like, the cycloalkyl group may be substituted; aryl group such as phenyl, naphthyl and the like, the aryl group may be substituted; heteroaryl group such as pyridyl, thienyl, pyrrolyl, furyl and the like, the heteroaryl group may be substituted; heteroaralkyl group such as furanmethyl, pyridinemethyl, oxazolemethyl, oxazolethyl and the like, the heteroaralkyl group may be substituted; aralkyl group such as benzyl, phenethyl and the like, the aralkyl group may be substituted; heterocyclyl group such as aziridinyl, pyrrolidinyl, piperidinyl and the like, the heterocyclyl group may be substituted. The substituents on R³ may be selected from halogen, hydroxy, nitro or unsubstituted or substituted groups selected from alkyl, cycloalkyl, alkoxy, cycloalkoxy, aryl, aralkyl, aralkoxyalkyl, heterocyclyl, heteroaryl, heteroaralkyl, alkanoyl, alkanoyloxy, hydroxyalkyl, amino, alkanoylamino, arylamino, aminoalkyl, aryloxy, aralkoxy, alkoxycarbonyl, alkylamino, alkoxyalkyl, alkylthio, thioalkyl groups, carboxylic acid or its derivatives such as amides, like CONH₂, CONHMe, CONMe₂, CONHEt, CONEt₂, CONHPh and the like, or esters such as COOCH₃, COOC₂H₅, COOC₃H₇ and the like, the carboxylic acid derivatives may be substituted; sulfonic acid or its derivatives such as SO₂NH₂, SO₂NHMe, SO₂NMe₂, SO₂NHCF₃ and the like, or sulfonates such as mesylate, tosylate, triflate, OSO₂C₂H₅ and the like, the sulfonic acid derivatives may be substituted.

Suitable groups represented by R⁴, R⁵ and R⁶ may be selected from hydrogen, substituted or unsubstituted linear or branched (C₁-C₁₆)alkyl, preferably (C₁-C₁₂)alkyl group such as methyl, ethyl, n-propyl, i-propyl, n-butyl, i-butyl, pentyl, hexyl, heptyl, octyl and the like; hydroxy(C₁-C₆)alkyl such as hydroxymethyl, hydroxyethyl, hydroxybutyl and the like; aryl group such as phenyl, naphthyl and the like, the aryl group may be substituted; aralkyl group such as benzyl, phenethyl, phenyl propyl, phenyl butyl, phenyl pentyl, phenyl hexyl, phenyl heptyl and the like, the aralkyl group may be substituted; carboxyalkyl group such as carboxymethyl, carboxyethyl, carboxypropyl and the like, which may be substituted; (C₂-C₆)alkanoyl group such as acetyl, propanoyl, butanoyl, pentanoyl, benzoyl and the like, which may be substituted; aralkanoyl group such as phenyl acetyl, naphthyl acetyl and the like, which may be substituted; aroyl such as benzoyl, substituted benzoyl and the like; alkanoyloxy group such as OOCMe, OOCEt, OOCPh and the like which may be substituted; heterocyclyl group such as aziridinyl, pyrrolidinyl, piperidinyl and the like; heteroaryl group such as pyridyl, thienyl, pyrrolyl, furyl and the like; heteroaralkyl group such as furanmethyl, pyridinemethyl, oxazolemethyl, oxazolethyl and the like; (C₁-C₁₂)alkylcarboxylic acid such as methyl carboxylic acid, ethyl carboxylic acid, propyl carboxylic acid, butyl carboxylic acid, hexy carboxylic acid, heptyl carboxylic acid and the like and their derivatives such as esters or amides. The substituents may be selected from halogen, hydroxy, nitro or substituted or unsubstituted groups selected from alkyl, cycloalkyl, alkoxy, cycloalkoxy, aryl, aralkyl, aryloxy, aralkoxy, aralkoxyalkyl, heterocyclyl, heteroaryl, heteroaralkyl, hydroxyalkyl, amino, arylamino, aminoalkyl, alkylamino, alkoxyalkyl, alkylthio, thioalkyl groups, carboxylic acid or its derivatives such as amides, like CONH₂, CONHMe, CONMe₂, CONHBEt, CONEt₂, CONHPh and the like, or esters such as COOCH₃, COOC₂H₅, COOC₃H₇ and the like, the carboxylic acid derivatives may be substituted; sulfonic acid or its derivatives such as SO₂NH₂, SO₂NHMe, SO₂NMe₂, SO₂NHCF₃ and the like, or sulfonates such as mesylate, tosylate, triflate, OSO₂C₂H₅ and the like. The sulfonic acid derivatives may be substituted.

Suitable ring structures formed by R³ and R⁴ together may be selected from pyrrolidinyl, piperidinyl morpholinyl, piperazinyl and the like.

Suitable groups represented by R⁷ may be selected from hydrogen, hydroxy, halogen atom such as fluorine, chlorine, bromine or iodine; lower alkyl groups such as methyl, ethyl, propyl and the like; (C₁-C₃)alkoxy such as methoxy, ethoxy, propoxy and the like; substituted or unsubstituted aralkyl such as benzyl, phenethyl and the like or R⁷ together with R¹ represents a bond. When the aralkyl group is substituted, the substituents may be selected from hydroxy, halogen atom, nitro or amino groups.

Suitable groups represented by R⁸, R⁹ and R¹⁰ may be selected from hydrogen, halogen atom such as fluorine, chlorine, bromine, or iodine; hydroxy, cyano, nitro, formyl; substituted or unsubstituted (C₁-C₁₂)alkyl group, especially, linear or branched (C₁-C₁₀)alkyl group, such as methyl, ethyl, n-propyl, i-propyl, n-butyl, i-butyl, t-butyl, n-pentyl, i-pentyl, hexyl, heptyl, octyl and the like; cyclo(C₃-C₆)alkyl group such as cyclopropyl, cyclobutyl, cyclopentyl cyclohexyl and the like, the cycloalkyl group may be substituted; (C₁-C₆)alkoxy such as methoxy, ethoxy, propyloxy, butyloxy, iso-propyloxy and the like, the alkoxy group may be substituted; cyclo(C₃-C₆)alkoxy group such as cyclopropyloxy, cyclobutyloxy, cyclopentyloxy, cyclohexyloxy and the like, the cycloalkoxy group may be substituted; aryl group such as phenyl, naphthyl and the like, the aryl group may be substituted; aryloxy group such as phenoxy, naphthyloxy and the like, the aryloxy group may be substituted; aralkyl such as benzyl, phenethyl, C₆H₅CH₂CH₂CH₂, naphthylmethyl and the like, the aralkyl group may be substituted; aralkoxy group such as benzyloxy, phenethyloxy, naphthylmethyloxy, phenylpropyloxy and the like, the aralkoxy group may be substituted; heterocyclyl groups such as aziridinyl, pyrrolidinyl morpholinyl piperidinyl, piperazinyl and the like, the heterocyclyl group may be substituted; heteroaryl group such as pyridyl, thienyl, furyl, pyrrolyl, oxazolyl, thiazolyl, imidazolyl, oxadiazolyl, tetrazolyl, benzopyranyl, benzofuranyl and the like, the heteroaryl group may be substituted; heteroaralkyl group such as furanmethyl, pyridinemethyl oxazolemethyl, oxazolethyl and the like, the heteroaralkyl group may be substituted; heteroaryloxy and heteroaralkoxy, wherein heteroaryl and heteroaralkyl moieties are as defined earlier and may be substituted; alkanoyl group such as acetyl, propanoyl and the like, the alkanoyl group may be substituted; aroyl such as benzoyl substituted benzoyl and the like; alkanoyloxy group such as OOCMe, OOCEt, OOCPh and the like which may be substituted; hydroxy(C₁-C₆)alkyl, which may be substituted; amino; alkanoylamino groups such as NHCOCH₃, NHCOC₂H₅, NHCOC₃H₇, NHCOC₆H₅, which may be substituted; mono(C₁-C₆)alkylamino group such as NHCH₃, NHC₂H₅, NHC₃H₇, NHC₆H₁₃ and the like, which may be substituted; (C₁-C₆)dialkylamino group such as N(CH₃)₂, NCH₃(C₂H₅) and the like, which may be substituted; arylamino group such as HNC₆H₅, NCH₃(C₆H₅), NHC₆H₄CH₃, NHC₆H₄-Hal and the like, which may be substituted; aralkylamino group such as C₆H₅CH₂NH, C₆H₅CH₂CH₂NH, C₆H₅CH₂NCH₃ and the like, which may be substituted; amino(C₁-C₆)alkyl, which may be substituted; alkoxycarbonyl such as methoxycarbonyl, ethoxycarbonyl and the like, which may be substituted; aryloxycarbonyl group such as phenoxycarbonyl, naphthyloxycarbonyl and the like, which may be substituted; aralkoxycarbonyl group such as benzyloxycarbonyl, phenethyloxycarbonyl, naphthylmethoxycarbonyl and the like, which may be substituted; alkoxyalkyl group such as methoxymethyl, ethoxymethyl, methoxyethyl, ethoxyethyl and the like, the alkoxyalkyl groups may be substituted; aryloxyalkyl group such as C₆H₅OCH₂, C₆H₅OCH₂CH₂, naphthyloxymethyl and the like, which may be substituted; aralkoxyalkyl group such as C₆H₅CH₂OCH₂, C₆H₅CH₂OCH₂CH₂ and the like, which may be substituted; thio(C₁-C₆)alkyl such as thiomethyl, thioethyl and the like, which may be substituted; (C₁-C₆)alkylthio such as methylthio, ethylthio and the like, which may be substituted; alkoxycarbonylamino group such as NHCOOC₂H₅, NHCOOCH₃ and the like, which may be substituted; aryloxycarbonylamino group such as NHCOOC₆H₅, N(CH₃)COOC₆H₅, N(C₂H₅)COOC₆H₅, NHCOOC₆H₄CH₃, NHCOOC₆H₄OCH₃ and the like, which may be substituted; aralkoxycarbonylamino group such as NHCOOCH₂C₆H₅, NHCOOCH₂CH₂C₆H₅, N(CH₃)COOCH₂C₆H₅, N(C₂H₅)COOCH₂C₆H₅, NHCOOCH₂C₆H₄CH₃, NHCOOCH₂C₆H₄OCH₃ and the like, which may be substituted; carboxylic acid or its derivatives such as amides, like CONH₂, CONHMe, CONMe₂, CONHEt, CONEt₂, CONHPh and the like, or esters such as COOCH₃, COOC₂H₅, COOC₃H₇ and the like, the carboxylic acid derivatives may be substituted; sulfonic acid or its derivatives such as SO₂NH₂, SO₂NHMe, SO₂NMe₂, SO₂NHCF₃, or sulfonates such as mesylate, tosylate, triflate, OSO₂C₂H₅ and the like.

When the groups represented by R⁸, R⁹ and R¹⁰ are substituted, the substituent may be selected from halogen, hydroxy, nitro or substituted or unsubstituted groups selected from alkyl, cycloalkyl, alkoxy, cycloalkoxy, aryl, aralkyl, aryloxy, aralkoxy, aralkoxyalkyl, heterocyclyl, heteroaryl, heteroaralkyl, hydroxyalkyl, amino, arylamino, aminoalkyl, alkylamino, alkoxyalkyl, alkylthio, thioalkyl groups, carboxylic acid or sulfonic acid.

Suitable groups represented by Ar may be selected from substituted or unsubstituted groups selected from divalent phenylene, naphthylene, pyrrolyl, pyridyl, quinolinyl, benzofuryl, dihydrobenzofuryl, benzopyranyl, dihydrobenzopyranyl, indolyl, indolinyl, azaindolyl, azaindolinyl, pyrazolyl, benzothiazolyl, benzoxazolyl and the like. The substituents on the group represented by Ar may be selected from linear or branched optionally halogenated (C₁-C₆)alkyl, optionally halogenated (C₁-C₃)alkoxy, halogen, acyl, amino, acylamino, thio or carboxylic or sulfonic acids and their derivatives. The substituents are defined as they are for R⁸-R¹⁰.

It is more preferred that Ar represent substituted or unsubstituted divalent, phenylene, naphthylene, benzofuryl, pyrrolyl, indolyl, indolinyl, quinolinyl, azaindolyl, azaindolinyl, benzothiazolyl or benzoxazolyl groups.

Suitable n is an integer ranging from 1-4.

Suitable m and p are integers ranging from 04.

Pharmaceutically acceptable salts forming part of this invention include salts derived from inorganic bases such as Li, Na, K, Ca, Mg, Fe, Cu, Zn, Al, Mn; salts of organic bases such as N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, theobromine, glycinol, diethylamine, triethylamine, trimethylamine, tripropylamine, tromethamine, adamentyl amine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, thiamine, aminopyrimidine, aminopyridine, purine, spermidine, and the like; chiral bases like alkylphenylamine, phenyl glycinol and the like, salts of natural amino acids such as glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids such as D-isomers or substituted amino acids; guanidine, substituted guanidine wherein the substituents are selected from nitro, amino, alkyl, alkenyl, alkynyl, ammonium or substituted ammonium salts. Salts may include acid addition salts where appropriate which are, sulphates, nitrates, phosphates, perchlorates, borates, hydrohalides, acetates, tartrates, maleates, citrates, succinates, palmoates, methanesulfonates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

Particularly useful compounds according to the present invention include:
Ethyl 2-[4-(5-ethyl-1-methyl-3-propyl-1H-pyrazolo[4,3-d]pyrimidin-7-yloxymethyl(heptyl)carboxamido)phenylsulfanyl]pentanoate or its salts in its single enantiomeric form or as a racemate;
2-[4-5-Ethyl-1-methyl-3-propyl-1H-pyrazolo[4,3-d]pyrimidin-7-yloxymethyl-(heptyl)carboxamido)phenylsulfanyl]pentanoic acid or its salts in its single enantiomeric form or as a racemate;
Ethyl 2-ethoxy-3-[4-{2-(5-ethyl-1-methyl-3-propyl-1H-pyrazol[4,3-d]pyrimidin-7-yloxy)ethoxy}phenyl]propanoate or its salts in its single enantiomeric form or as a racemate;
Methyl 2-ethoxy-3-[4-{2-(5-ethyl-1-methyl-3-propyl-1H-pyrazolo[4,3-d]-pyrimidin-7-yloxy)ethoxy}phenyl]propanoate or its salts in its single enantiomeric form or as a racemate;
2-Ethoxy-3-[4-{2-(5-ethyl-1-methyl-3-propyl-1H-pyrazolo[4,3-d]pyrimidin-7-yloxy)ethoxy}phenyl]propanoic acid or its salts in its single enantiomeric form or as a racemate;
Ethyl 3-[4-{2-(1,5-dimethyl-3-propyl-1H-pyrazolo[4,3-d]pyrimidin-7-yloxy)ethoxy}phenyl]-2-ethoxypropanoate or its salts in its single enantiomeric form or as a racemate;
3-[4-{2-(1,5-Dimethyl-3-propyl-1H-pyrazolo[4,3-d]pyrimidin-7-yloxy)-ethoxy}phenyl]ethoxypropanoic acid or its salts in its single enantiomeric form or as a racemate;
Ethyl 2-ethoxy-3-[4-{2-(1-methyl-5-phenyl-3-propyl-1H-pyrazolo-[4,3-d]pyrimidin-7-yloxy)ethoxy}phenyl]propanoate or its salts in its single enantiomeric form or as a racemate;
3-[4-{2-(1-Methyl-5-phenyl-3-propyl-1H-pyrazolo-[4,3-d]pyrimidin-7-yloxy)ethoxy}phenyl]-2-ethoxypropanoic acid or its salts in its single enantiomeric form or as a racemate;
Ethyl 3-[4-{2-(5-cyclopropyl-1-methyl-3-propyl-1H-pyrazolo[4,3-d]pyrimidin-7-yloxy)ethoxy}phenyl]2-ethoxypropanoate or its salts in its single enantiomeric form or as a racemate;
3-[4-{2-5-Cyclopropyl-1-methyl-3-propyl-1H-pyrazolo[4,3-d]pyrimidin-7-yloxy)ethoxy}phenyl]-2-ethoxypropanoic acid or its salts in its single enantiomeric form or as a racemate;
Ethyl 6-[5-ethyl-1-methyl-3-propyl-1H-pyrrolo[4,3-d]pyrimidin-7-yloxy-methyl {4-(1-ethyloxycarbonylbutoxyl)phenyl}carboxamido]hexanoate or its salts in its single enantiomeric form or as a racemate;
6-[4-(1-Carboxybutoxy)phenyl(5-ethyl-1-methyl-3-propyl-1H-pyrazolo[4,3-d]pyrimidin-7-yloxymethyl)carboxamido]hexanoic acid or its salts in its single enantiomeric form or as a racemate;
Ethyl 3-[4-{1,5-dimethyl-3-propyl-1H-pyrazolo[4,3-d]pyrimidin-7-yloxy-methyl(4-fluorobenzyl)carboxamido}phenyl]-2-ethoxypropanoate or its salts in its single enantiomeric form or as a racemate;
3-[4-{1,5-Dimethyl-3-propyl-1H-pyrazolo[4,3-d]pyrimidin-7-yloxymethyl(4-fluorobenzyl)carboxamido}phenyl]-2-ethoxypropanoic acid or its salts in its single enantiomeric form or as a racemate;
Ethyl 2-(4-[5-ethyl-1-methyl-3-propyl-1H-pyrazolo[4,3-d]pyrimidin-7-yloxy-methyl {4-(4-ethyloxycarbonylphenyl)butyl}carboxamido]phenoxy)pentanoate or its salts in its single enantiomeric form or as a racemate;
2-[4-{4-(4-Carboxyphenyl)butyl(5-ethyl-1-methyl-3-propyl-1H-pyrazolo[4,3-d]pyrimidin-7-yloxymethyl)carboxamido}phenoxy]pentanoic acid or its salts in its single enantiomeric form or as a racemate;

According to a feature of the present invention, the compounds of general formula (I) where Y represents CHR⁷ group, R⁷ and R¹ together represent a bond, G is O or S; Z represents oxygen atom; and all other symbols are as defined earlier, can be prepared by any of the following routes shown in Scheme-I below.

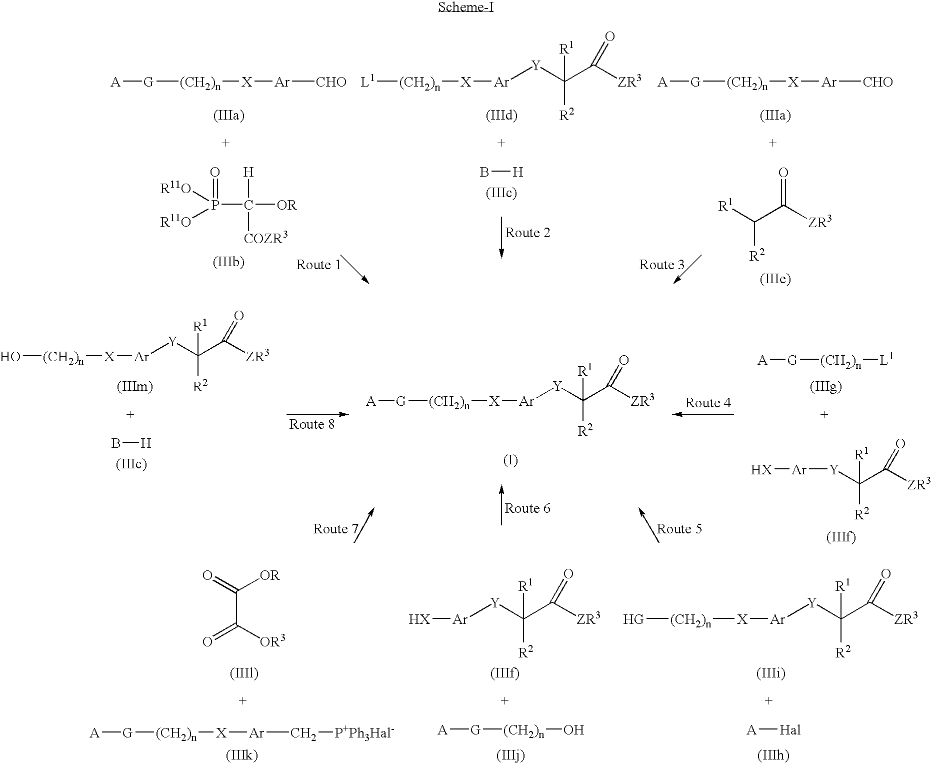

Route 1: The reaction of a compound of the general formula (IIIa) where all symbols are as defined earlier with a compound of formula (IIIb) where R¹¹ represents (C₁-C₆)alkyl, R represents substituted or unsubstituted groups selected from alkyl, aryl, aralkyl, heteroaryl or heteroaralkyl and all other symbols are as defined earlier to yield compound of general formula (I) where R² represents substituted or unsubstituted groups selected from alkoxy, aryloxy, aralkoxy, heteroaryloxy, heteroaralkoxy and all other symbols are as defined above may be carried out in the presence of a base such as alkali metal hydrides like NaH or KH; organolithiums such as LDA, TMEDA and the like; alkoxides such as NaOMe, NaOEt, t-BuO⁻K⁺ and the like or mixtures thereof. The reaction may be carried out in the presence of solvents such as THF, dioxane, DMF, DMSO, DME and the like or mixtures thereof. HMPA may be used as cosolvent. The reaction temperature may range from −78° C. to 50° C., preferably at a temperature in the range of −10° C. to 30° C. The reaction is more effective under anhydrous conditions. The compound of general formula (IIIb) may be prepared by Arbuzov reaction.

Alternatively, the compound of formula (I) may be prepared by reacting the compound of formula (IIIa) where all symbols are as defined earlier with Wittig reagents such as Hal⁻Ph₃P⁺CH—(OR)CO₂R³ under similar reaction conditions as described above.

Route 2: The compound of general formula (IIIc) where B represents pyrazolopyrimidine or imidazolopyrimidine of the formula given below: 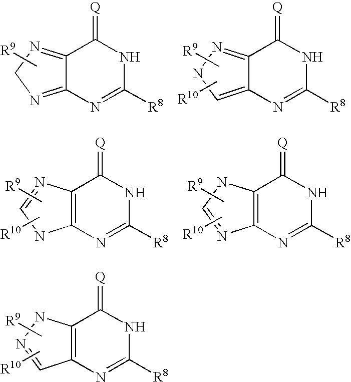
wherein Q represents O or S and all other symbols are as defined earlier, because of the keto-enol tautomersim, enol form reacts with compound of general formula (IIId) where L¹ is a leaving group such as halogen atom, p-toluenesulfonate, methanesulfonate, trifluoromethanesulfonate and the like; R¹ and R⁷ together represent a bond and all other symbols are as defined earlier to produce compound of formula (I) where all symbols are as defined above. The reaction may be carried out in the presence of solvents such as DMSO, DMF, DME, THF, dioxane, ether and the like or mixtures thereof. The reaction may be carried out in an inert atmosphere that may be maintained by using inert gases such as N₂, argon, He and the like. The reaction may be effected in the presence of a base such as alkalis like sodium hydroxide or potassium hydroxide; alkali metal carbonates such as sodium carbonate or potassium carbonate; alkali metal hydrides such as sodium hydride or potassium hydride; organometallic bases like n-butyl lithium; alkali metal amides like sodamide, organic base like triethyl amine or mixtures thereof. The amount of base may range from 1 to 5 equivalents, based on the amount of the compound of formula (IIIc), preferably the amount of base ranges from 1 to 3 equivalents. Phase transfer catalysts such as tetraalkylammonium halide or hydroxide may be added. Additives such as alkali metal halides such as LiBr may be added. The reaction may be carried out at a temperature in the range of 0° C.-150° C., preferably at a temperature in the range of 15° C.-100° C. The duration of the reaction may range from 0.25 to 72 hours, preferably from 0.25 to 24 hours.

Route 3: The reaction of a compound of the general formula (IIIa) where all symbols are as defined earlier, with a compound of formula (IIIe) where R¹ represents hydrogen atom and all other symbols are as defined earlier may be carried out in the presence of a base. The nature of the base is not critical. Any base normally employed for aldol condensation reaction may be employed; bases like metal hydride such as NaH, KH, metal alkoxides such as NaOMe, t-BuO⁻K⁺, NaOEt metal amides such as LiNH₂, LiN(ipr)₂ may be used. Aprotic solvents such as THF, ether, dioxane may be used. The reaction may be carried out in an inert atmosphere that may be maintained by using inert gases such as N₂, Ar, or He and the reaction is more effective under anhydrous conditions. Temperature in the range of −80° C. to 35° C. may be used. The β-hydroxy product initially produced may be dehydrated under conventional dehydration conditions such as treating with p-TSA in solvents such as benzene or toluene. The nature of solvent and dehydrating agent is not critical. Temperature in the range of 20° C. to reflux temperature of the solvent used may be employed, preferably at reflux temperature of the solvent by continuous removal of water using a Dean-Stark water separator.

Route 4: The reaction of compound of formula (IIIg) where L¹ represents a leaving group such as halogen atom, p-toluenesulfonate, methanesulfonate, trifluoromethanesulfonate ard the like; and all other symbols are as defined earlier with compound of formula (IIIf) where R¹ and R⁷ together represent a bond and all other symbols are as defined earlier to produce a compound of the formula (I) defined above may be carried out in the presence of aprotic solvents such as THF, DMF, DMSO, DME and the like or mixtures thereof. The reaction may be carried out in an inert atmosphere that may be maintained by using inert gases such as N₂, Ar, He and the like. The reaction may be effected in the presence of a base such as K₂CO₃, Na₂CO₃ or NaH or mixtures thereof. Acetone may be used as solvent when Na₂CO₃ or K₂CO₃ is used as a base. The reaction temperature may range from 0° C.-120° C., preferably at a temperature in the range of 30° C.-100° C. The duration of the reaction may range from 1 to 48 hours, preferably from 2 to 12 hours. The compound of formula (IIIf) can be prepared according to a known procedure by Wittig Homer reaction between the hydroxy protected aryl aldehyde such as benzyloxyaryl aldehyde and compound of formula (IIIb), followed by deprotection.

Route 5: The reaction of compound of general formula (IIIh) where A is as defined earlier and Hal represents halogen atom such as chlorine, bromine, fluorine or iodine, with compound of formula (IIIi) where R¹ and R⁷ together represent a bond and all other symbols are as defined earlier to yield the compound of general formula (I) where all symbols are as defined above may be carried out in the presence of a base such as alkali metal hydrides like NaH or KH; organolithiums such as TMEDA, LDA and the like; alkoxides such as NaOMe, NaOEt, t-BuO⁻K⁺ and the like or mixtures thereof. The reaction may be carried out in the presence of solvents such as THF, dioxane, DMF, DMSO, DME and the like or mixtures thereof. The reaction temperature may range from −78° C. to 120° C., preferably at a temperature in the range of −10° C. to 80° C. The reaction is more effective under anhydrous conditions.

Route 6: The reaction of compound of general formula (IIIj) where all symbols are as defined earlier with a compound of general formula (ED where R¹ and R⁷ together represent a bond and all other symbols are as defined earlier may be carried out using suitable coupling agents such as EDCI, dicyclohexyl urea, triarylphosphine/dialkylazadicarboxylate such as PPh₃/DEAD or DIAD and the like. The reaction may be carried out in the presence of solvents such as THF, DME, CH₂Cl₂, CHCl₃, toluene, acetonitrile, carbon tetrachloride and the like. The inert atmosphere may be maintained by using inert gases such as N₂, Ar, He and the like. The reaction may be effected in the presence of DMAP, HOBt and they may be used in the range of 0.05 to 2 equivalents, preferably 0.25 to 1 equivalents. The reaction temperature may be in the range of −20° C. to 100° C., preferably at a temperature in the range of 0° C. to 80° C. The duration of the reaction may range from 0.5 to 24 hours, preferably from 6 to 12 hours. The above condensation may also be made using mixed anhydride methodology.

Route 7: The reaction of a compound of formula (IIIk) where all symbols are as defined earlier with a compound of formula (IIIl) where R represents substituted or unsubstituted groups selected from alkyl, aryl, aralkyl, heteroaryl or heteroaralkyl and where R³ is as defined earlier excluding hydrogen to yield compound of general formula (I) where R² represents substituted or unsubstituted groups selected from alkoxy, aryloxy, aralkoxy, heteroaryloxy, heteroaralkoxy and all other symbols are as defined above may be carried out neat in the presence of a base such as alkali metal hydrides like NaH, KH or organolithiums like CH₃Li, BuLi, TMEDA, LDA and the like or alkoxides such as NaOMe, NaOEt, t-BuO⁻K⁺ and the like or mixtures thereof. The reaction may be carried out in the presence of aprotic solvents such as THF, dioxane, DMF, DMSO, DME and the like or mixtures thereof. HMPA may be used as cosolvent. The reaction temperature may range from −78° C. to 100° C., preferably at a temperature in the range of −10° C. to 50° C. The reaction is more effective under anhydrous condition.

Route 8: The compound of general formula (IIIc) where B represents pyrazolopyrimidine or imidazolopyrimidine of the formula given below: 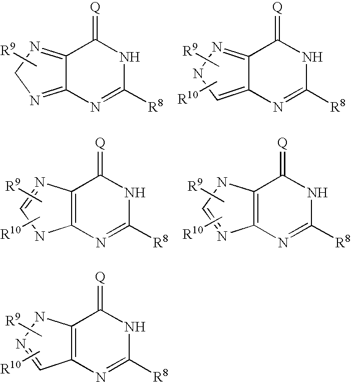
wherein Q represents O or S and all other symbols are as defined earlier, because of the keto-enol tautomersim, enol form reacts with compound of general formula (IIIm) which represents compound of formula (IIIi) when G represents oxygen, R¹ and R⁷ together represent a bond and all other symbols are as defined earlier to produce compound of formula (I) where all symbols are as defined above. The reaction may be carried out using suitable coupling agents such as EDCI, dicyclohexyl urea, triarylphosphine/dialkylazadicarboxylate such as PPh₃/DEAD or DIAD and the like. The reaction may be carried out in the presence of solvents such as THF, DME, CH₂Cl₂, CHCl₃, toluene, acetonitrile, carbon tetrachloride and the like. The inert atmosphere may be maintained by using inert gases such as N₂, Ar, He and the like. The reaction may be effected in the presence of DMAP, HOBt and they may be used in the range of 0.05 to 2 equivalents, preferably 0.25 to 1 equivalents. The reaction temperature may be in the range of −20° C. to 100° C., preferably at a temperature in the range of 0° C. to 80° C. The duration of the reaction may range from 0.5 to 120 hours, preferably from 6 to 24 hours.

In yet another embodiment of the present invention, the compounds of general formula (I) where Y represent CHR⁷ group, wherein R⁷ represents hydrogen atom, halogen, hydroxy, alkyl, alkoxy, substituted or unsubstituted aralkyl group; R¹ represents hydrogen atom, hydroxy, alkoxy, halogen, lower alkyl, substituted or unsubstituted aralkyl group; X, R², R³, n, Ar and A are as defined earlier, G is O or S and Z represents oxygen atom can be prepared by one or more of the processes shown in Scheme-II below:

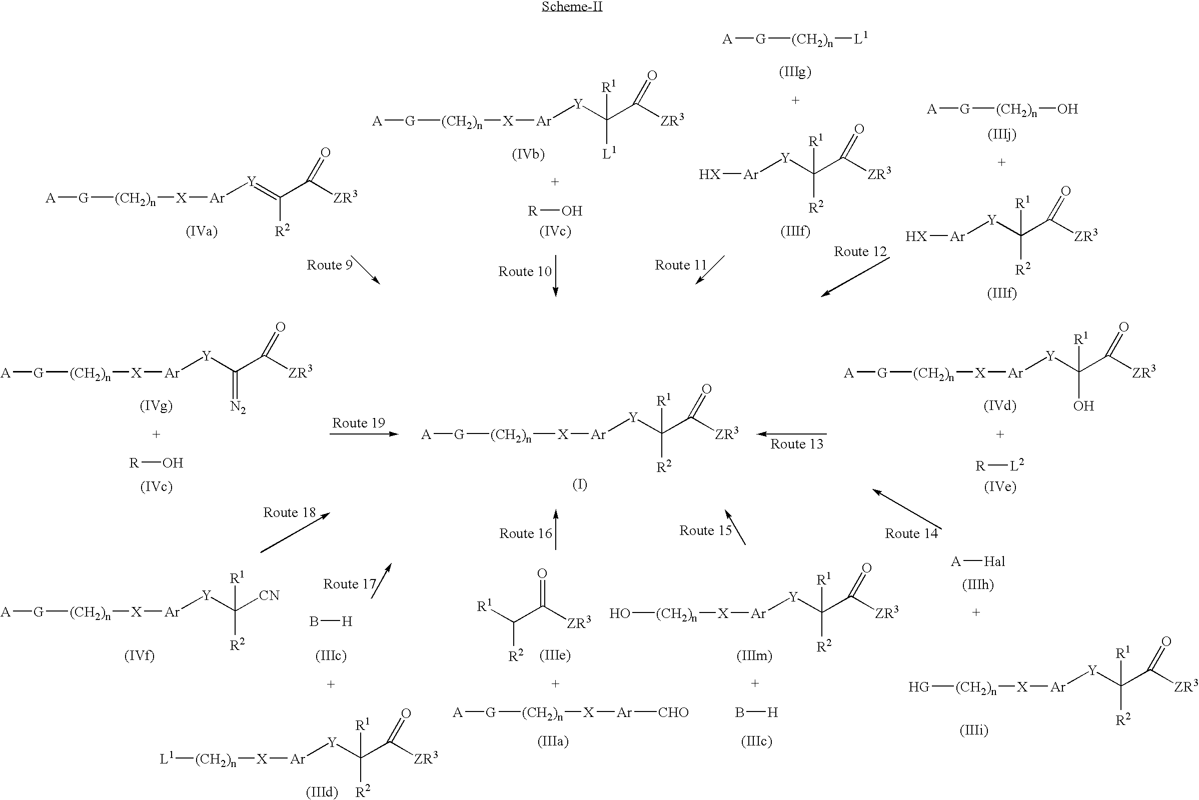

Route 9: The reduction of compound of the formula (IVa) which represents a compound of formula (I) where R¹ and R⁷ together represent a bond and Z represents oxygen atom and all other symbols are as defined earlier, obtained as described earlier (Scheme-I), to yield a compound of the general formula (I) where R¹ and R⁵ each represent hydrogen atom and all symbols are as defined earlier, may be carried out in the presence of gaseous hydrogen and a catalyst such as Pd(OH)₂/C, Pd/C, Rh/C, Pt/C, Ra—Ni, and the like. Mixtures of catalysts may be used. The reaction may be conducted in the presence of solvents such as dioxane, acetic acid, ethyl acetate, alcohol such as methanol, ethanol and the like. A pressure between atmospheric pressure and 80 psi may be employed. The catalyst may be preferably 5-10% Pd/C and the amount of catalyst used may range from 5-100% w/w. The reaction may also be carried out by employing metal solvent reduction such as magnesium in alcohol or sodium amalgam in alcohol, preferably methanol. The reaction temperature may range from 20° C.-120° C., preferably at a temperature in the range of 25° C.-100° C. The duration of the reaction may range from 1 to 48 hours, preferably from 2 to 6 hours. The hydrogenation may be carried out in the presence of metal catalysts containing chiral ligands to obtain a compound of formula (I) in optically active form. The metal catalyst may contain Rhodium, Ruthenium, Indium and the like. The chiral ligands may preferably be chiral phosphines such as optically pure enantiomers of 2,3-bis(diphenylphosphino)butane, 1,2-bis(diphenylphosphino)ethane, 1,2-bis(2-methoxyphenyl phenylphosphino)ethane, 2,3-isopropylidene-2,3-dihydroxy-1,4-bis(diphenylphosphino)butane and the like. Any suitable chiral catalyst may be employed which would give required optical purity of the product (I) (Ref: Principles of Asymmetric Synthesis, Tetrahedron Series Vol 14, pp 311-316, Ed. Baldwin J. E.).

Route 10: The reaction of compound of formula (IVb) where L¹ is a leaving group such as halogen atom, p-toluenesulfonate, methanesulfonate, trifluoromethanesulfonate and the like, R³ is as defined earlier excluding hydrogen and all other symbols are as defined earlier with an alcohol of general formula (IVc), where R represents substituted or unsubstituted groups selected from alkyl, aryl, aralkyl, heteroaryl or heteroaralkyl and all other symbols are as defined earlier to yield compound of general formula (I) where R² represents substituted or unsubstituted groups selected from alkoxy, aryloxy, aralkoxy, heteroaryloxy, heteroaralkoxy and all symbols earlier may be carried out in the presence of solvents such as TNF, DMF, DMSO, DME and the like or mixtures thereof. The reaction may be carried out in an inert atmosphere that may be maintained by using inert gases such as N₂, Ar, He and the like. The reaction may be effected in the presence of a base such as KOH, NaOH, NaOMe, NaOEt, t-BuO⁻K⁺ or NaH or mixtures thereof. Phase transfer catalysts such as tetraalkyl ammonium halides or bisulphates or hydroxides may be employed. The reaction temperature may range from 20° C.-120° C., preferably at a temperature in the range of 30° C.-100° C. The duration of the reaction may range from 1 to 24 hours, preferably from 2 to 6 hours.

Route 11: The reaction of compound of formula (IIIg) defined earlier with compound of formula (IIIf) where all symbols are as defined earlier to produce a compound of the formula (I) defined above, may be carried out in the presence of solvents such as THF, DMF, DMSO, DME and the like or mixtures thereof. The reaction may be carried out in an inert atmosphere that is maintained by using inert gases such as N₂, Ar, He and the like. The reaction may be effected in the presence of a base such as Na₂CO₃, K₂CO₃, NaH and the like or mixtures thereof. Acetone may be used as a solvent when Na₂CO₃ or K₂CO₃ is used as a base. The reaction temperature may range from 20° C.-120° C., preferably at a temperature in the range of 30° C.-80° C. The duration of the reaction may range from 1 to 48 hours, preferably from 2 to 12 hours. The compound of formula (IIIf) may be prepared by using Wittig Homer reaction between the protected hydroxyaryl aldehyde and compound of formula (IIIb) followed by reduction of the double bond and deprotection.

Route 12: The reaction of compound of general formula (IIIj) defined earlier with a compound of general formula (IIIf) where all symbols are as defined above may be carried out using suitable coupling agents such as EDCI, dicyclohexyl urea, triarylphosphine/dialkylazadicarboxylate such as PPh₃/DEAD or DIAD and the like. The reaction may be carried out in the presence of solvents such as THF, DME, CH₂Cl₂, CHCl₃, toluene, acetonitrile, carbon tetrachloride and the like. The inert atmosphere may be maintained by using inert gases such as N₂, Ar, He and the like. The reaction may be effected in the presence of DMAP, HOBt and they may be used in the range of 0.05 to 2 equivalents, preferably 0.25 to 1 equivalents. The reaction temperature may be in the range of −20° C. to 100° C., preferably at a temperature in the range of 0° C. to 80° C. The duration of the reaction may range from 0.5 to 24 hours, preferably from 6 to 12 hours. The above condensation may also be made using mixed anhydride methodology.

Route 13: The reaction of compound of formula (IVd), which represents a compound of formula (I), when R² represents hydroxy group and all other symbols are as defined above with a compound of formula (IVe) where R represents substituted or unsubstituted groups selected from alkyl, aryl, aralkyl, heteroaryl or heteroaralkyl and L² is a halogen atom to yield compound of general formula (I) where R² represents substituted or unsubstituted groups selected from alkoxy, aryloxy, aralkoxy, heteroaryloxy, heteroaralkoxy and all symbols are as defined earlier may be carried out in the presence of solvents such as THF, DMF, DMSO, DME and the like. The inert atmosphere may be maintained by using inert gases such as N₂, Ar, He and the like. The reaction may be effected in the presence of a base such as KOH, NaOH, NaOMe, t-BuO⁻K⁺, NaH, KH and the like. Phase transfer catalyst such as tetraalkyl ammonium halides or bisulphates or hydroxides may be employed. The reaction temperature may range from 20° C. to 150° C., preferably at a temperature in the range of 25° C. to 100° C. The duration of the reaction may range from 1 to 24 hours, preferably from 2 to 6 hours.

Route 14: The reaction of compound of general formula (IIIh) where Hal represents halogen atom and A is as defined earlier with the compound of formula (IIIi) where all other symbols are as defined earlier to yield the compound of general formula (I) where all symbols are as defined above may be carried out in the presence of a base such as alkali metal hydrides like NaH or KH; organolithiums such as TMEDA, LDA and the like; alkoxides such as NaOMe, NaOEt, t-BuO⁻K⁺ and the like or mixtures thereof. The reaction may be carried out in the presence of solvents such as THF, dioxane, DMF, DMSO, DME and the like or mixtures thereof. The reaction temperature may range from −78° C. to 120° C., preferably at a temperature in the range of −10° C. to 80° C. The reaction is more effective under anhydrous conditions.

Route 15: The compound of general formula (IIIc) where B represents pyrazolopyrimidine or imidazolopyrimidine of the formula given below: 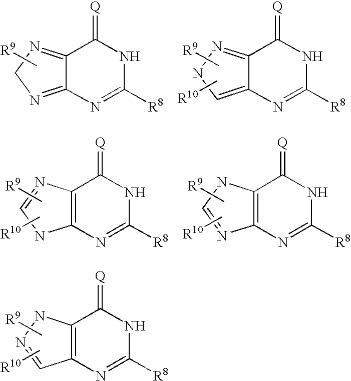
wherein Q represents O or S and all other symbols are as defined earlier, because of the keto-enol tautomersim, enol form reacts with reacts with compound of general formula (IIIm) which represents compound of formula (IIIi) when G is oxygen and all other symbols are as defined earlier to produce compound of formula (I) where all symbols are as defined above. The reaction may be carried out using suitable coupling agents such as EDCI, dicyclohexyl urea, triarylphosphine/dialkylazadicarboxylate such as PPh₃/DEAD or DIAD and the like. The reaction may be carried out in the presence of solvents such as THF, DME, CH₂Cl₂, CHCl₃, toluene, acetonitrile, carbon tetrachloride and the like. The inert atmosphere may be maintained by using inert gases such as N₂, Ar, He and the like. The reaction may be effected in the presence of DMAP, HOBt and they may be used in the range of 0.05 to 2 equivalents, preferably 0.25 to 1 equivalents. The reaction temperature may be in the range of 0° C. to 100° C., preferably at a temperature in the range of 20° C. to 80° C. The duration of the reaction may range from 0.5 to 120 hours, preferably from 6 to 24 hours.

Route 16: The reaction of a compound of the general formula (IIIa) as defined above with a compound of formula (IIIe) where R¹ represents hydrogen atom and all other symbols are as defined earlier may be carried out in the presence of base. The base is not critical. Any base normally employed for aldol condensation reaction may be employed, metal hydride such as NaH or KH; metal alkoxides such as NaOMe, t-BuO⁻K^{t} or NaOEt; metal amides such as LiNH₂, LiN(iPr)₂. Aprotic solvent such as THF may be used. Inert atmosphere may be employed such as N₂ or argon or He and the reaction is more effective under anhydrous conditions. Temperature in the range of −80° C. to 25° C. may be used. The β-hydroxy aldol product may be dehydroxylated using conventional methods, conveniently by ionic hydrogenation technique such as by treating with a trialkyl silane in the presence of an acid such as trifluoroacetic acid. Solvent such as CH₂Cl₂ may be used. Favorably, reaction proceeds at 25° C. Higher temperature may be employed if the reaction is slow.

Route 17: The compound of general formula (IIIc) where B represents pyrazolopyrimidine or imidazolopyrimidine of the formula given below: 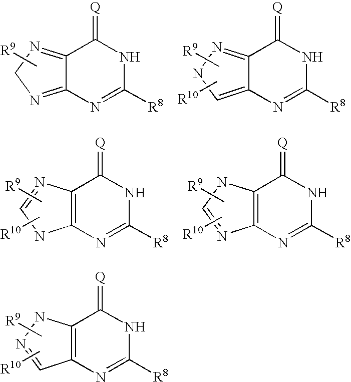
wherein Q represents O or S and all other symbols are as defined earlier, because of the keto-enol tautomersim, enol form reacts with reacts with compound of general formula (IIId) where L¹ is a leaving group such as halogen atom, p-toluenesulfonate, methanesulfonate, trifluoromethanesulfonate and the like; all other symbols are as defined earlier to produce compound of formula (I) where all symbols are as defined above. The reaction may be carried out in the presence of solvents such as DMSO, DMF, DME, THF, dioxane, ether and the like or a combination thereof. The reaction may be carried out in an inert atmosphere that may be maintained by using inert gases such as N₂, Ar, He and the like. The reaction may be effected in the presence of a base such as alkalis like sodium hydroxide, potassium hydroxide; alkali metal carbonates like sodium carbonate or potassium carbonate; alkali metal hydrides such as sodium hydride or potassium hydride; organometallic bases like LDA, TMEDA; alkali metal amides like sodamide or mixtures thereof. The amount of base may range from 1 to 5 equivalents, based on the amount of the compound of formula (IIIc), preferably the amount of base ranges from 1 to 3 equivalents. Additives such as alkali metal halides such as LiBr may be added. The reaction may be carried out at a temperature in the range of 0° C. to 150° C., preferably at a temperature in the range of 15° C. to 100° C. The duration of the reaction may range from 0.25 to 48 hours, preferably from 0.25 to 24 hours.

Route 18: The conversion of compound of formula (IVf) where all symbols are as defined earlier to a compound of formula (I) may be carried out either in the presence of base or acid and the selection of base or acid is not critical. Any base normally used for hydrolysis of nitrile to acid may be employed, metal hydroxides such as NaOH or KOH in an aqueous solvent or any acid normally used for hydrolysis of nitrile to ester may be employed such as HCl in an excess of alcohol such as methanol, ethanol, propanol etc. The reaction may be carried out at a temperature in the range of 0° C. to reflux temperature of the solvent used, preferably at a temperature in the range of 25° C. to reflux temperature of the solvent used. The duration of the reaction may range from 0.25 to 48 hrs.

Route 19: The reaction of a compound of formula (IVg) where R³ is as defined earlier excluding hydrogen and all other symbols are as defined earlier with a compound of formula (IVc) where R represents substituted or unsubstituted groups selected from alkyl, aryl, aralkyl, heteroaryl or heteroaralkyl and L² is a halogen atom to yield compound of general formula (I) where R² represents substituted or unsubstituted groups selected from alkoxy, aryloxy, aralkoxy, heteroaryloxy, heteroaralkoxy and all other symbols are as defined earlier (by a rhodium carbenoid mediated insertion reaction) may be carried out in the presence of rhodium (II) salts such as rhodium (II) acetate. The reaction may be carried out in the presence of solvents such as benzene, toluene, dioxane, ether, THF and the like or a combination thereof or when practicable in the presence of ROH as solvent at any temperature providing a convenient rate of formation of the required product, generally at an elevated temperature, such as reflux temperature of the solvent. The inert atmosphere may be maintained by using inert gases such as N₂, Ar, He and the like. The duration of the reaction may range from 0.5 to 24 h, preferably from 0.5 to 6 h.

In yet another embodiment of the present invention the compound of general formula (I) where Y represents O or S or NR⁶ group where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; X represents NHR⁵, —CO(CH₂)ₚNR⁵(CH₂)ₘ—, —(CH₂)ₚO—, —(CH₂)ₚNR⁵CO— where m and p are as defined earlier; G is O or S and Z represents oxygen atom; R¹, R², R³, A, n and Ar are as defined earlier, can be prepared by any of the following routes shown in Scheme-III below.

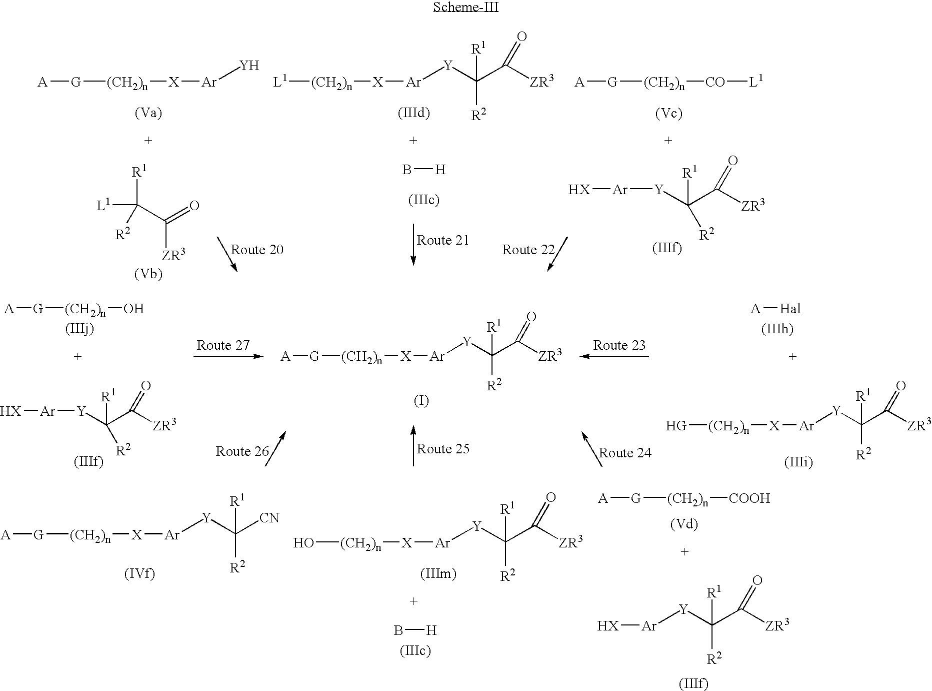

Route 20: The reaction of compound of formula (Va) where all symbols are as defined earlier with compound of formula (Vb) where L¹ is a leaving group such as halogen atom, p-toluenesulfonate, methanesulfonate, trifluoromethanesulfonate and the like, and all other symbols are as defined earlier to produce a compound of the formula (I) defined above may be carried out in the presence of aprotic solvents such as THF, DMF, DMSO, DME and the like; organic base like triethyl amine, lutidine, collidine and the like or mixtures thereof. The reaction may be carried out in an inert atmosphere that may be maintained by using inert gases such as N₂, Ar, He and the like. The reaction may be effected in the presence of a base such as K₂CO₃, Na₂CO₃, NaH, KH and the like or mixtures thereof. Acetone may be used as solvent when Na₂CO₃ or K₂CO₃ is used as a base. The reaction temperature may range from 0° C.-120° C., preferably at a temperature in the range of 25° C.-100° C. The duration of the reaction may range from 1 to 96 hours, preferably from 2 to 24 hours.

Route 21: The reaction of a compound of general formula (IIIc) where B represents pyrazolopyrimidine or imidazolopyrimidine of the formula given below: 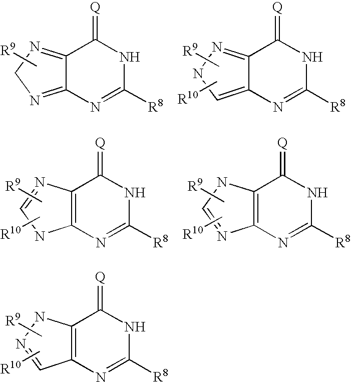
wherein Q represents O or S and all other symbols are as defined earlier, because of the keto-enol tautomerism, enol form reacts with reacts with compound of general formula (IIId) where L¹ is a leaving group such as halogen atom, p-toluenesulfonate, methanesulfonate, trifluoromethanesulfonate and the like, and all other symbols are as defined earlier to produce a compound of general formula (I) where all symbols are as defined above may be carried out in the presence of solvents such as DMSO, DMF, DME, THF, dioxane, ether, acetone and the like or mixtures thereof. The reaction may be carried out in an inert atmosphere that may be maintained by using inert gases such as N₂, argon, He and the like. The reaction may be effected in the presence of a base such as alkalis like sodium hydroxide or potassium hydroxide; alkali metal carbonates such as sodium carbonate or potassium carbonate; alkali metal hydrides such as sodium hydride or potassium hydride; organometallic bases like n-BuLi, TMEDA (N,N,N′,N′-tetramethylethylenediamine), LDA; alkali metal amides like sodamide, organic base like triethyl amine, lutidine, collidine or mixtures thereof. The amount of base may range from 1 to 5 equivalents, based on the amount of the compound of formula (IIIc), preferably the amount of base ranges from 1 to 3 equivalents. Phase transfer catalysts such as tetraalkylammonium halide or sulphonates or hydroxide may be added. Additives such as alkali metal halides like LiBr may be added. The reaction may be carried out at a temperature in the range of 0° C.-150° C., preferably at a temperature in the range of 15° C.-100° C. The duration of the reaction may range from 0.25 to 72 hours, preferably from 0.25 to 24 hours.

Route 22: The reaction of compound of formula (Vc) where L¹ represents a leaving group such as halogen atom, p-toluenesulfonate, methanesulfonate, trifluoromethanesulfonate and the like, and all other symbols are as defined earlier with compound of formula (IIIf) where all symbols are as defined earlier to produce a compound of the formula (I) defined above may be carried out in the presence of aprotic solvents such as THF, DMF, DMSO, DME and the like or mixtures thereof. The reaction may be carried out in an inert atmosphere that may be maintained by using inert gases such as N₂, Ar, He and the like. The reaction may be effected in the presence of a base such as K₂CO₃, Na₂CO₃ or NaH, KH, triethyl amine and the like or mixtures thereof. Acetone may be used as solvent when Na₂CO₃ or K₂CO₃ is used as a base. The reaction temperature may range from 0° C.-120° C., preferably at a temperature in the range of 25° C.-100° C. The duration of the reaction may range from 1 to 72 hours, preferably from 2 to 24 hours.

Route 23: The reaction of compound of general formula (IIIh) where A is as defined earlier and Hal represents halogen atom with the compound of formula (IIIi) where all symbols are as defined earlier to yield compound of general formula (I) where all symbols are as defined above may be carried out in the presence of a base such as alkali metal hydrides like NaH or KH; organolithiums such as TMEDA, LDA and the like; alkoxides such as NaOMe, NaOEt, t-BuO⁻K⁺ and the like or mixtures thereof. The reaction may be carried out in the presence of solvents such as THF, dioxane, DMF, DMSO, DME and the like or mixtures thereof. The reaction temperature may range from −78° C. to 120° C., preferably at a temperature in the range of −10° C. to 80° C. The reaction is more effective under anhydrous conditions.

Route 24: The reaction of compound of formula (Vd) where all symbols are as defined earlier with a compound of general formula (IIIf) where all symbols are as defined earlier may be carried out using pivaloyl chloride, ethyl chloroformate, isobutyl chloroformate and the like. The reaction may be carried out in the presence of solvents such as CH₂Cl₂, DMF, THF and the like or mixtures thereof. The inert atmosphere may be maintained by using inert gases such as N₂, Ar, He and the like. The reaction may be carried out in the presence of bases like triethyl amine, lutidine, collidine and the like. The reaction temperature may be in the range of −20° C. to 80° C., preferably at a temperature in the range of 0° C. to 50° C. The duration of the reaction may range from 0.5 to 24 hours, preferably from 0.5 to 12 hours.

This reaction can also be carried out by a method described in Route 25.

Route 25: The reaction of compound of general formula (IIIc) where B represents pyrazolopyrimidine or imidazolopyrimidine of the formula given below: 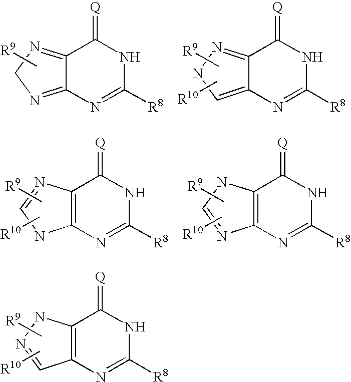
wherein Q represents O or S and all other symbols are as defined earlier, because of the keto-enol tautomerism, enol form reacts with reacts with compound of general formula (IIIm) where all symbols are as defined earlier to afford compound of general formula (I) where all symbols are as defined earlier may be carried out using suitable coupling agents such as EDCI (N-ethyl-N′-3-dimethylaminopropyl)carbodimide hydrochloride), dicyclohexyl urea, triarylphosphine/dialkylazadicarboxylate such as PPh₃/DEAD or DIAD and the like. The reaction may be carried out in the presence of solvents such as THF, DME, CH₂Cl₂, CHCl₃, toluene, acetonitrile, carbon tetrachloride and the like. The inert atmosphere may be maintained by using inert gases such as N₂, Ar, He and the like. The reaction may be effected in the presence of DMAP, HOBt and they may be used in the range of 0.05 to 2 equivalents, preferably 0.25 to 1 equivalents. The reaction temperature may be in the range of −20° C. to 100° C., preferably at a temperature in the range of 0° C. to 80° C. The duration of the reaction may range from 0.5 to 48 hours, preferably from 6 to 18 hours.

Route 26: The conversion of compound of formula (IVf) where all symbols are as defined earlier to a compound of formula (I) may be carried out either in the presence of base or acid and the selection of base or acid is not critical. Any base normally used for hydrolysis of nitrile to acid may be employed, metal hydroxides such as NaOH or KOH in an aqueous solvent or any acid normally used for hydrolysis of nitrile to ester may be employed such as HCl in an excess of alcohol such as methanol, ethanol, propanol etc. The reaction may be carried out at a temperature in the range of 0° C. to reflux temperature of the solvent used, preferably at a temperature in the range of 25° C. to reflux temperature of the solvent used. The duration of the reaction may range from 0.25 to 48 hrs.

Route 27: The reaction of compound of general formula (IIIj) where A, G and n are as defined earlier with a compound of general formula (IIIf) where all symbols are as defined earlier may be carried out using suitable coupling agents such as EDCI, dicyclohexyl urea, triarylphosphine/dialkylazadicarboxylate such as PPh₃/DEAD or DIAD and the like. The reaction may be carried out in the presence of solvents such as THF, DME, CH₂Cl₂, CHCl₃, toluene, acetonitrile, carbon tetrachloride and the like. The inert atmosphere may be maintained by using inert gases such as N₂, Ar, He and the like. The reaction may be effected in the presence of DMAP, HOBt and they may be used in the range of 0.05 to 2 equivalents, preferably 0.25 to 1 equivalents. The reaction temperature may be in the range of −20° C. to 100° C., preferably at a temperature in the range of 0° C. to 80° C. The duration of the reaction may range from 0.5 to 48 hours, preferably from 6 to 18 hours. The above condensation may also be made using mixed anhydride methodology as described in Route 24.

The compound of formula (I) where R³ represents hydrogen atom may be prepared by hydrolysing, using conventional methods, a compound of formula (I) where R³ represents all groups defined earlier excluding hydrogen. The hydrolysis may be carried out in the presence of a base such as Na₂CO₃, K₂CO₃, NaOH, KOH, LiOH and the like and a suitable solvent such as methanol, ethanol, dioxane, water and the like or mixtures thereof THF is used as solubilizing agent wherever necessary. The reaction may be carried out at a temperature in the range of 20° C. to reflux temperature of the solvent, preferably at 25° C. to reflux temperature. The reaction time may range from 1 to 48 h, preferably from 1 to 12 h.

The compound of general formula (I) where Z represents oxygen and R³ represents hydrogen or lower alkyl group may be converted to compound of formula (I), where Z represents NR⁴ by reaction with appropriate amines of the formula NHR³R⁴, where R³ and R⁴ are as defined earlier to yield a compound of formula (I) where Z represents NR⁴ and all other symbols are as defined earlier. Alternatively, the compound of formula (I) where ZR³ represents OH may be converted to acid halide, preferably ZR³═Cl by reacting with appropriate reagents such as oxalyl chloride, thionyl chloride and the like, followed by treatment with amines of the formula NHR³R⁴ where R³ and R⁴ are as defined earlier. Alternatively, mixed anhydrides may be prepared from compound of formula (I) where ZR³ represents OH and all other symbols are as defined earlier by treating with acid halides such acetyl chloride, acetyl bromide, pivaloyl chloride, dichlorobenzoyl chloride and the like. The reaction may be carried out in the presence of pyridine, triethylamine, diisopropyl ethylamine and the like. Coupling reagents such as DCC/DMAP, DCC/HOBt, EDCI/HOBt, ethylchloroformate, isobutylchloroformate can also be used to activate the acid. Solvents such as halogenated hydrocarbons like CHCl₃ or CH₂Cl₂; hydrocarbons such as benzene, toluene, xylene and the like may be used. The reaction may be carried out at a temperature in the range of −40° C. to 40° C., preferably at a temperature in the range of 0° C. to room temperature. The acid halide or mixed anhydride or activated acid obtained by coupling reagents described above thus prepared may further be treated with appropriate amines of the formula NHR³R⁴ where R³ and R⁴ are as defined earlier to yield a compound of formula (I) where Z represents NR⁴ and all other symbols are as defined earlier.

In still another embodiment of the present invention the novel intermediate of formula (IIIf) 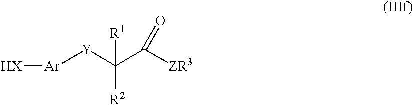
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4 is provided.

In yet another embodiment of the present invention, the compound of formula (IIIf) where X represents NHR⁵, —CO(CH₂)ₚNR⁵(CH₂)ₘ—, —(CH₂)ₚNR⁵CO—, where p is 0, m is 0-4, R⁵ is as defined above; Y represents O, S, NR⁶ or CHR⁷ all other symbols are as defined above may be prepared by any of the following the processes described in scheme-IV below:

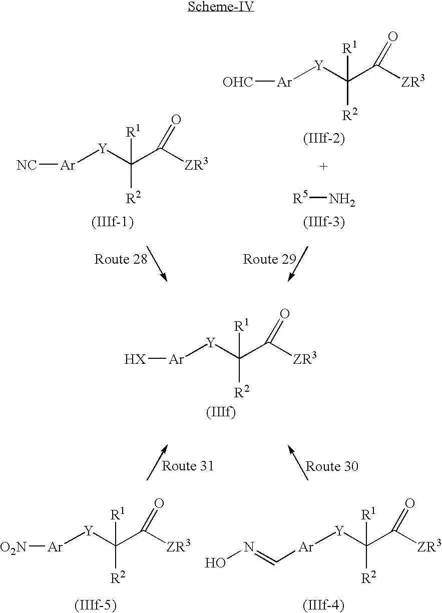

Route 28: The reduction of compound of the formula (IIIf-1) where all symbols are as defined above to yield compound of the general formula (IIIf) where R⁵ represents hydrogen; and all other symbols are as defined above may be carried out in the presence of gaseous hydrogen and a catalyst such as Pd/C, Rh/C, Pt/C, and the like. Mixtures of catalysts may be used. The reaction may also be carried out in the presence of solvents such as dioxane, acetic acid, ethyl acetate, alcohol such as methanol, ethanol and the like or mixture thereof. A pressure between atmospheric pressure and 40 to 80 psi may be employed. The catalyst may be preferably 5-10% Pd/C and the amount of catalyst used may range from 5-100% w/w. The reaction may also be carried out by employing metal solvent reduction such as magnesium or samarium in alcohol or sodium amalgam in alcohol, preferably methanol. When R¹ and R² represent double bond, the hydrogenation may be carried out in the presence of metal catalysts containing chiral ligands to obtain a compound of formula (IIIf) in optically active form. The metal catalyst may contain Rhodium, Ruthenium, Indium and the like. The chiral ligands may preferably be chiral phosphines such as optically pure enantiomers of 2,3-bis(diphenylphosphino)butane, 2,3-isopropylidene-2,3-dihydroxy-1,4-bis(diphenylphosphino)butane and the like. Any suitable chiral catalyst may be employed which would give required optical purity of the product (I) (Ref: Principles of Asymmetric Synthesis, Tetrahedron Series Vol 14, pp 311-316, Ed. Baldwin J. E.).

Route 29: The reaction of compound of formula (IIIf-2) with compound of formula (IIIf-3) to give a compound of formula (IIIf) where R⁵ represents alkyl group and all other symbols are as defined above may be carried out in two steps the first step being the imine formation, followed by reduction. Formation of imine may be carried out in solvents such as MeOH, EtOH, i-PrOH and the like. The reaction may be effected in the presence of a promoter such as NaOAc, KOAc and the like or the mixtures thereof. The temperature of reaction may range from room temperature to the reflux temperature of the solvent used. The reaction time may be 2 h to 24 h, preferably in the range 2 h to 12 h.

The imine can also be obtained by the reaction of a compound of general formula (IIIf-2) with a compound of formula (IIIf-3) using solvents such as CH₂Cl₂, CHCl₃, chlorobenzene, benzene, THF, in the presence of catalyst such as p-toluenesulfonic acid, methanesulfonic acid, TFA, TfOH, BF₃—OEt₂ and the like. The reaction may also be carried out in presence of activated molecular sieves. The temperature of the reaction may range from 10° C. to 100° C., preferably at a temperature in the range from 10° C. to 60° C. The reaction time may be 1 h to 48 h.

The imine product thus obtained above may be reduced by using Na(CN)BH₃—HCl (ref: Hutchins, R. O. et al. J. Org. Chem. 1983, 48, 3433), NaBH₁, H₂—Pd]/C, H₂—t/C; H₂—Rh/C and the like in solvents such as methanol, ethanol and the like.

Route 30: The conversion of compound of formula (IIIf-4) to a compound of formula (IIIf) where R⁵ represents hydrogen, and all other symbols are as defined above may be carried out in two steps the first step being the imine formation, followed by reduction. Formation of imine may be carried out in solvents such as MeOH, EtOH, i-PrOH and the like using hydroxylamine hydrochloride. The reaction may be effected in the presence of a promoter such as NaOAc, KOAc and the like or the mixtures thereof. The temperature of reaction may range from room temperature to the reflux temperature of the solvent used. The reaction time may be 2 h to 24 h, preferably in the range 2 h to 12 h.

The imine product thus obtained above may be reduced by using Na(CN)BH₃—HCl (ref: Hutchins, R. O. et al. J. Org Chem. 1983, 48, 3433), NaBH₄, H₂—Pd]/C, H₂—Pt/C, H₂—Rh/C and the like in solvents such as methanol, ethanol and the like.

Alternatively, the conversion of compound of formula (IIIf-4) to a compound of formula (IIIf) where R⁵ represents hydrogen, and all other symbols are as defined above may be carried out using TiCl₃ as reagent and a reducing agent such as NaCNBH₃, or Na₂BH₄. The coactivators like ammonium acetate can be used. The reaction may be carried out in the presence of solvents such as methanol, ethanol water and the like. The temperature of reaction may range from −20° C. to the reflux temperature of the solvent used. The reaction time may be 1 h to 48.

The compound of formula (IIIf-4) where all symbols are as defined earlier can be converted to corresponding methane sulfonate, p-toluene sulfonate, trifluoromethane sulfonate and the like. The intermediates thus obtained are then subjected to hydrogenation following the condition as described in route 7 and route 15 to yield compound of formula (IIIf) where all symbols are as defined above.

Route 31: The reduction of compound of the formula (IIIf-5) to yield compound of the general formula (IIIf) where R⁵ represents hydrogen and all other symbols are as defined above may be carried out in the presence of gaseous hydrogen and a catalyst such as Pd/C, Rh/C, Pt/C, Raney nickel and the like. Mixtures of catalysts may be used. The reaction may also be conducted in the presence of solvents such as dioxane, acetic acid, ethyl acetate and the like. A pressure between atmospheric pressure and 80 psi may be employed. The catalyst may be preferably 5-10% Pd/C and the amount of catalyst used may range from 1-50% w/w. The reaction may also be carried out by employing metal solvent reduction such as magnesium, iron, tin, samarium in alcohol or sodium amalgam in alcohol, preferably methanol or ethanol. The hydrogenation may be carried out in the presence of metal catalysts containing chiral ligands to obtain a compound of formula (IIIf) in optically active form. The metal catalyst may contain Rhodium, Ruthenium, Indium and the like. The chiral ligands may preferably be chiral phosphines such as optically pure enantiomers of 2,3-bis(diphenylphosphino)butane, 2,3-isopropylidene-2,3-dihydroxy-1,4-bis(diphenylphosphino)butane and the like. (Ref: Principles of Asymmetric Synthesis, Tet. Org. Chem Series Vol 14, pp 311-316, Ed. Baldwin J. E.).

In yet another embodiment of the present invention, the compound of formula (IIIf) where R² represents substituted or unsubstituted groups selected from alkoxy, aryloxy, aralkoxy, heteroaryloxy, heteroaralkoxy; X represents NHR⁵, Y represents O, S, NR⁶ or CHR⁷, Z is as defined above excluding NH and all other symbols are as defined above may be prepared by diazotizing the compound of formula (IIIf-6) to a compound of formula (IIIf-7) and reducing the compound of formula (IIIf-7) to yield compound of formula (IIIf). The reaction shown in scheme-V below:

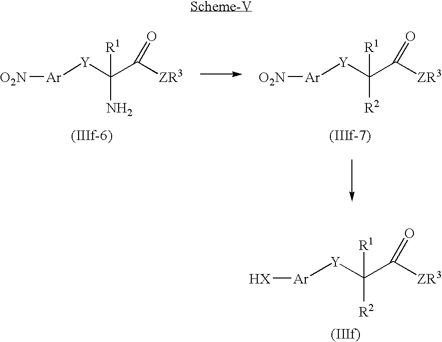

The diazotization of the compound of the formula (IIIf-6) where Z is as defined above excluding NH and all other symbols are as defined above to obtain compound of formula (IIIf-7) where R² represents substituted or unsubstituted groups selected from alkoxy, aryloxy, aralkoxy, heteroaryloxy, heteroaralkoxy and all other symbols are as defined earlier may be carried out using diazotizing agent such as sodium nitrite, isoamyl nitrite, potassium nitrite, ammonium nitrite and the like under aqueous acidic conditions using acids such as sulfuric acid, HCl, acetic acid and the like, in an organic solvent such as alcohols such as methanol, ethanol, propanol and the like; 1,4-dioxane, THF, acetone and the like. Etherifying the residue obtained using alkyl sulfates such as diethyl sulphate, dimethylsulphate and the like or alkyl halides such as ethyl iodide, methyliodide and the like, in solvents such as hydrocarbons like toluene, benzene and the like or DMF, DMSO, methyl isobutyl ketone (MIBK) and the like, in the presence of alkali bases such as sodium carbonate, potassium carbonate, sodium methoxide, sodium hydride, potassium hydride and the like.

The reduction of compound of the formula (IIIf-7) to yield a compound of the general formula (IIIf) where R⁵ represents hydrogen atom and all other symbols are as defined earlier may be carried out in the presence of gaseous hydrogen and a catalyst such as Pd/C, Rh/C, Pt/C, Raney nickel and the like. Mixtures of catalysts may be used. The reaction may also be conducted in the presence of solvents such as dioxane, acetic acid, ethyl acetate and the like. A pressure between atmospheric pressure and 80 psi may be employed. The catalyst may be preferably 5-10% Pd/C and the amount of catalyst used may range from 1-50% w/w. The reaction may also be carried out by employing metal solvent reduction such as magnesium, iron, tin, samarium in alcohol or sodium amalgam in alcohol, preferably methanol. The hydrogenation may also be carried out using ammonium formate, cyclohex-1,4-diene type of hydrogen donor under pd/c conditions using solvents such as methanol, ethanol, ethyl acetate and the like.

In yet another embodiment of the present invention, the compound of formula (IIIf) where R² represents substituted or unsubstituted groups selected from alkoxy, aryloxy, aralkoxy, heteroaryloxy, heteroaralkoxy, X represents NHR⁵, Z is as defined above excluding NH and all other symbols are as defined above may be prepared by diazotizing and reducing the compound of formula (IIIf-6) to a compound of formula (IIIf-8) and etherifying the compound of formula (IIIf-8) to yield compound of formula (IIIf). The reaction shown in scheme-VI below:

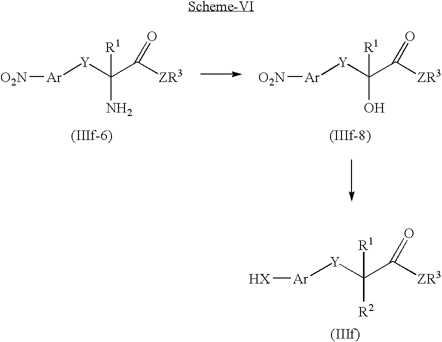

The diazotization of the compound of the formula (IIIf-6) where all symbols are as defined above to obtain compound of formula (IIIf-8) may be carried out using diazotizing agent such as sodium nitrite, isoamyl nitrite, potassium nitrite, ammonium nitrite and the like under aqueous acidic conditions using acids such as sulfuric acid, HCl, acetic acid and the like, in an organic solvent such as alcohols such as methanol, ethanol, propanol and the like; 1,4-dioxane, THF, acetone and the like. Reducing the residue obtained using gaseous hydrogen and a catalyst such as Pd/C, Rh/C, Pt/C, Raney nickel and the like. Mixtures of catalysts may be used. The reduction may also be conducted in the presence of solvents such as dioxane, acetic acid, ethyl acetate and the like. A pressure between atmospheric pressure and 80 psi may be employed. The catalyst may be preferably 5-10% Pd/C and the amount of catalyst used may range from 1-50% w/w. The reaction may also be carried out by employing metal solvent reduction such as magnesium, iron, tin, samarium in alcohol or sodium amalgam in alcohol, preferably methanol. The hydrogenation may also be carried out using ammonium formate, cyclohex-1,4-diene type of hydrogen donor under pd/c conditions using solvents such as methanol, ethanol, ethyl acetate and the like.

The Etherification of compound of formula (IIIf-8) to yield compound of formula (IIIf) where R² represents substituted or unsubstituted groups selected from alkoxy, aryloxy, aralkoxy, heteroaryloxy, heteroaralkoxy and all other symbols are as defined earlier may be carried out using alkyl sulfates such as diethyl sulphate, dimethylsulphate and the like or alkyl halides such as ethyl iodide, methyliodide and the like, in solvents such as hydrocarbons like toluene, benzene and the like or DMF, DMSO, methyl isobutyl ketone (MIBK) and the like, in the presence of alkali bases such as sodium carbonate, potassium carbonate, sodium methoxide, sodium hydride, potassium hydride and the like.

In yet another embodiment of the present invention, the compound of formula (IIIf) where R² represents hydroxy or substituted or unsubstituted groups selected from alkoxy, aryloxy, aralkoxy, heteroaryloxy, heteroaralkoxy; X represents NHR⁵, Z is as defined above excluding NH and all other symbols are as defined above may be prepared by following the process described in scheme-VII below:

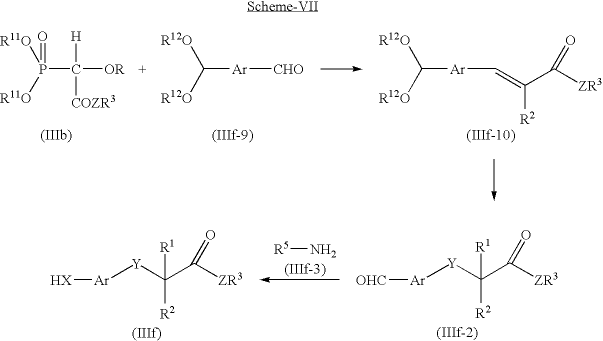

The reaction of a compound of the general formula (IIIb) where all symbols are as defined above with a compound of formula (IIIf-9) where R¹² represents (C₁-C₆)alkyl group to yield compound of formula (IIIf-10) may be carried out in the presence of a base such as metal hydride like NaH or KH; organolithiums such as CH₃Li, BuLi and the like; alkoxides such as NaOMe, NaOEt, t-BuO⁻K⁺ and the like or mixtures thereof. The reaction may be carried out in the presence of solvents such as diethyl ether, THF, dioxane, DMF, DMSO, DME, dimethyl acetamide and the like or mixtures thereof HMPA may be used as cosolvent. The reaction temperature may range from −78° C. to 50° C., preferably at a temperature in the range of −10° C. to 30° C.

The reduction of compound of the formula (IIIf-10) to yield a compound of the formula (IIIf-2) may be carried out in the presence of gaseous hydrogen and a catalyst such as Pd/C, Rh/C, Pt/C, Raney nickel and the like. Mixtures of catalysts may be used. The reaction may also be conducted in the presence of solvents such as dioxane, acetic acid, ethyl acetate and the like. A pressure between atmospheric pressure and 80 psi may be employed The catalyst may be preferably 5-10% Pd/C and the amount of catalyst used may range from 1-50% w/w. The reaction may also be carried out by employing metal solvent reduction such as magnesium, iron, tin, samarium in alcohol or sodium amalgam in alcohol, preferably methanol followed by an acidic work up. The hydrogenation may be carried out in the presence of metal catalysts containing chiral ligands to obtain a compound of formula (I) in optically active form. The metal catalyst may contain Rhodium, Ruthenium, Indium and the like. The chiral ligands may preferably be chiral phosphines such as optically pure enantiomers of 2,3-bis(diphenylphosphino)butane, 2,3-isopropylidene-2,3-dihydroxy-1,4-bis(diphenylphosphino)butane and the like.

The reaction of a compound of general formula (IIIf-2) with a compound of formula (IIIf-3) where R⁵ is as defined above to yield compound of formula (IIIf) may be carried out using solvents such as CH₂Cl₂, CHCl₃, chlorobenzene, benzene, THF, in the presence of catalyst such as p-toluenesulfonic acid, methanesulfonic acid, TFA, TfOH, BF₃—OEt₂ and the like. The reaction may also be carried out using activated molecular sieves. The temperature of the reaction may range from 10° C. to 100° C., preferably at a temperature in the range from 10° C. to 60° C. The, imine product initially produce may be reducing using Na(CN)BH₃—HCl (ref: Hutchins, R. O. et al. J. Org. Chem. 1983, vol. 48, 3433-3428), H₂—Pd/C, H₂—Pt/C, H₂—Ph/C and the like in solvents such as methanol, ethanol and the like.

Alternatively, the reaction of compound of formula (IIIf-2) to yield compound of formula (IIIf) where R⁵ represents hydrogen, m is 1, and all other symbols are as defined above may be carried out using substituted or unsubstituted hydroxylamine followed by treating with TiCl₃ as reagent and a reducing agent such as NaCNBH₃, or NaBH₄. The coactivators like ammonium acetate can be used. The reaction may be carried out in the presence of solvents such as methanol, ethanol, water and the like or mixtures thereof. The temperature of reaction may range from −20° C. to the reflux temperature of the solvent used. The reaction time may be 1 h to 48.

Alternatively, the reaction of compound of formula (IIIf-2) to yield compound of formula (IIIf) where R⁵ represents hydrogen, m is 1, and all other symbols are as defined above may be carried out using NH₃/methanol, NH₃/ethanol or NH₃/isopropanol in presence of Pd/C, Ra—Ni and the like as catalyst and atmospheric pressure to 80 psi pressure of hydrogen gas. The reaction temperature may range from room temperature to 60° C. The duration of reaction may range from 2 h to 48.

In still another embodiment of the present invention the novel intermediate of formula (IIId) 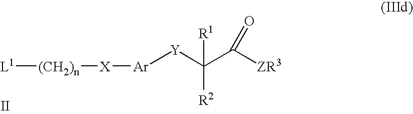
their derivatives, their analogs, their tautomeric forms, their stereoisomers, their polymorphs, their pharmaceutically acceptable salts, their pharmaceutically acceptable solvates where L¹ is a leaving group such as halogen atom, p-toluenesulfonate, methanesulfonate, trifluoromethanesulfonate; R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; n is an integer in the range of 1-4; m and p are integers ranging from 0-4 is provided.

In yet another embodiment of the present invention, the compound of formula (IIId) where X represents —CO(CH₂)ₚNR⁵(CH₂)ₘ—, —(CH₂)ₚNR⁵CO—; all symbols are as defined above may be prepared by a process which comprises, reacting the compound of formula (IIIf) 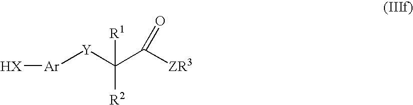
where X represents NHR⁵ and all other symbols are as defined above with compound of formula (IIIId-1)
L¹-(CH₂)ₙ—CO-L² (IIId-1)
where L¹ and n are as defined above and L² represents halogen atom.

The reaction of compound of formula (IIIf) with compound of formula (IIId-1) where may be carried out in the presence of solvents such as DCM, DCE, THF, DMF, DMSO, DME and the like or mixtures thereof. The reaction may be carried out in an inert atmosphere that may be maintained by using inert gases such as N₂, Ar, He and the like. The reaction may be effected in the presence of a base such as triethyl amine, lutidine, collidine and the like or mixtures thereof. The reaction temperature may range from −20° C.-120° C. The duration of the reaction may range from 1 to 48 hours, preferably from 2 to 12 hours.

In still another embodiment of the present invention the novel intermediate of formula (IIId) 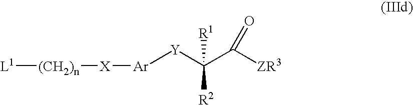
their derivatives, their analogs, their tautomeric forms, their polymorphs, their pharmaceutically acceptable salts, their pharmaceutically acceptable solvates where L¹ is a leaving group such as halogen, p-toluenesulfonate, methanesulfonate, trifluoromethanesulfonate; R¹ represents hydrogen; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from all, aryl, aralkyl, hydroxyalkyl, carboxyalkyl, alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; n is an integer in the range of 1-4; m and p are integers ranging from 0-4 is provided.

In yet another embodiment of the present invention, the compound of formula (IIId) where X represents —CO(CH₂)ₚNR⁵(CH₂)ₘ—, —(CH₂)NR⁵CO—; all symbols are as defined above may be prepared by a process which comprises, reacting the compound of formula (IIIf) 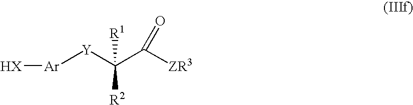
where X represents NHR⁵ and all other symbols are as defined above with compound of formula (IIId-1)
L¹-(CH₂)ₙ—CO-L² (IIId-1)
where L¹ and n are as defined above and L² represents halogen atom.

The reaction of compound of formula (IIIf) with compound of formula (IIId-1) where may be carried out in the presence of solvents such as DCM, DCE, THF, DMF, DMSO, DME and the like or mixtures thereof. The reaction may be carried out in an inert atmosphere that may be maintained by using inert gases such as N₂, Ar, He and the like. The reaction may be effected in the presence of a base such as triethyl amine, lutidine, collidine and the like or mixtures thereof. The reaction temperature may range from −20° C.-120° C. The duration of the reaction may range from 1 to 48 hours, preferably from 2 to 12 hours.

In still another embodiment of the present invention the novel intermediate of formula (IIId) 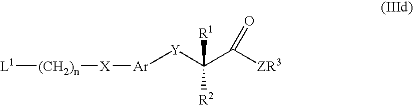
their derivatives, their analogs, their tautomeric forms, their polymorphs, their pharmaceutically acceptable salts, their pharmaceutically acceptable solvates where L¹ is a leaving group such as halogen, p-toluenesulfonate, methanesulfonate, trifluoromethanesulfonate; R¹ represents hydrogen; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; n is an integer in the range of 1-4; m and p are integers ranging from 0-4 is provided.

In yet another embodiment of the present invention, the compound of formula (IIId) where X represents —CO(CH₂)ₚNR⁵(CH₂)ₘ—, —(CH₂)ₚNR⁵CO—; all symbols are as defined above may be prepared by a process which comprises, reacting the compound of formula (IIIf) 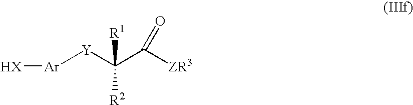
where X represents NHR⁵ and all other symbols are as defined above with compound of formula (IIId-1)
L¹-(CH₂)ₙ—CO-L² (IIId-1)
where L¹ and n are as defined above and L² represents halogen atom.

The reaction of compound of formula (IIIf) with compound of formula (IIId-1) where may be carried out in the presence of solvents such as DCM, DCE, THF, DMF, DMSO, DME and the like or mixtures thereof. The reaction may be carried out in an inert atmosphere that may be maintained by using inert gases such as N₂, Ar, He and the like. The reaction may be effected in the presence of a base such as triethyl amine, lutidine, collidine and the like or mixtures thereof. The reaction temperature may range from −20° C.-120° C. The duration of the reaction may range from 1 to 48 hours, preferably from 2 to 12 hours.

In still another embodiment of the present invention the novel intermediate of formula (IIIa)
A-G-(CH₂)ₙ—X—Ar—CHO (IIIa)
their derivatives, their analogs, their tautomeric forms, their stereoisomers, their polymorphs, their pharmaceutically acceptable salts, their pharmaceutically acceptable solvates where Ar represents substituted or unsubstituted, divalent, single or fused, aromatic, heteroaromatic or heterocyclic group; G represents O or S; X represents O, NHR⁵, —CO(CH₂)NR⁵(CH₂)ₘ—, (CH₂)ₚO, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; n is an integer in the range of 1-4; m and p are integers ranging from 0-4; A represents pyrazolopyrimidine or imidazolopyrimidine of the formula given below: 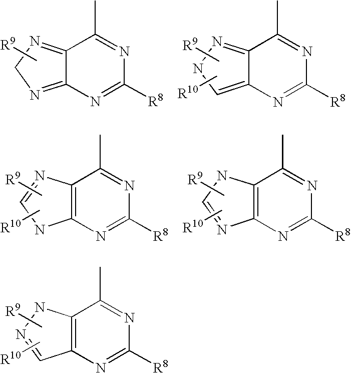
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives is provided.

In still another embodiment of the present invention the novel intermediate of formula (IVb) 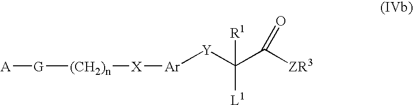
their derivatives, their analogs, their tautomeric forms, their stereoisomers, their polymorphs, their pharmaceutically acceptable salts, their pharmaceutically acceptable solvates where L¹ is a leaving group such as halogen atom, p-toluenesulfonate, methanesulfonate, trifluoromethanesulfonate and the like; R¹ represents hydrogen atom, halogen, hydroxy, alkyl, alkoxy, acyl, substituted or unsubstituted aralkyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, (CH₂)ₚO, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 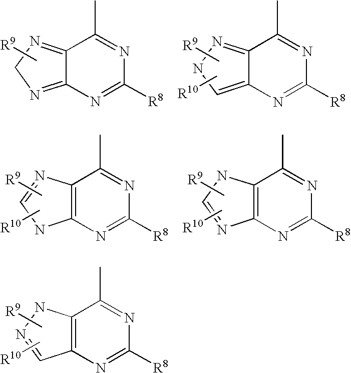
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives is provided.

In still another embodiment of the present invention the novel intermediate of formula (IVf) 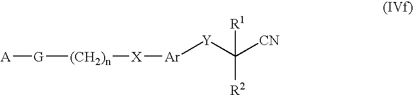
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; Ar represents substituted or unsubstituted, divalent, single or fused, aromatic, heteroaromatic, or heterocyclic group; G represents O or S; X represents O, NHR⁵, —CO(CH₂)ₚNR⁵(CH₂)—, (CH₂)ₚO, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 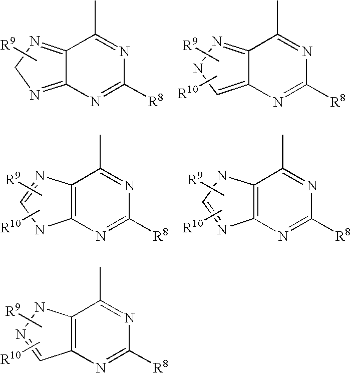
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl aralkyl, aryloxy, aralkoxy, heterocyclyl, heteroaryl, heteroaralkyl, heteroaryloxy, heteroaralkoxy, alkanoyl aroyl, alkanoyloxy, hydroxyalkyl, aminoalkyl, alkoxycarbonyl aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives is provided.

In still another embodiment of the present invention the novel intermediate of formula (IVg) 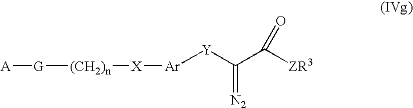
their derivatives, their analogs, their tautomeric forms, their stereoisomers, their polymorphs, their pharmaceutically acceptable salts, their pharmaceutically acceptable solvates where R³ represents hydrogen or substituted or unsubstituted groups selected from alkyl, cycloalkyl, aryl aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, (CH₂)ₚO, —(CH₂)ₚNR⁵CO—; where R⁵ represents hydrogen or substituted or unsubstituted groups selected from alkyl, aryl, alkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group; m and p are integers ranging from 0-4; n is an integer in the range of 1-4; A represents pyrazolopyrimidine or imidazolopyrimidine of the formula given below: 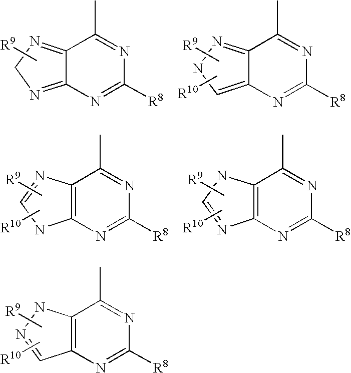
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives is provided.

It is appreciated that in any of the above mentioned reactions, any reactive group in the substrate molecule may be protected according to conventional chemical practice. Suitable protecting groups in any of the above mentioned reactions are tertiarybutyldimethylsilyl, methoxymethyl, triphenyl methyl, benzyloxycarbonyl, THP etc, to protect hydroxyl or phenolic hydroxy group; N-Poc, Boc, N-Cbz, N-Fmoc, benzophenoneimine etc, for protection of amino or anilino group, acetal protection for aldehyde, ketal protection for ketone and the like. The methods of formation and removal of such protecting groups are those conventional methods appropriate to the molecule being protected.

The pharmaceutically acceptable salts are prepared by reacting the compound of formula (I) with 1 to 4 equivalents of a base such as sodium hydroxide, sodium methoxide, sodium hydride, potassium hydroxide, potassium t-butoxide, calcium hydroxide, magnesium hydroxide and the like, in solvents like ether, THF, methanol, t-butanol, dioxane, isopropanol, ethanol, toluene etc. Mixtures of solvents may be used. Organic bases like lysine, arginine, diethanolamine, choline, guanidine, adamentyl amine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane and the like. Mixtures of solvents may also be used.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like wherever applicable or chiral bases such as brucine, cinchona alkaloids and their derivatives and the like. Commonly used methods are compiled by Jaques et al in “Enantiomers, Racemates and Resolution” (Wiley Interscience, 1981). More specifically the compound of formula (I) where ZR³ represents OH may be converted to a 1:1 mixture of diastereomeric amides by treating with chiral amines, aminoacids, aminoalcohols derived from aminoacids; conventional reaction conditions may be employed to convert acid into an amide; the diastereomers may, be separated either by fractional crystallization or chromatography and the stereoisomers of compound of formula (I) may be prepared by hydrolyzing the pure diastereomeric amide.

Various polymorphs of a compound of general formula (I) forming part of this invention may be prepared by crystallization of compound of formula (I) under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The present invention provides a pharmaceutical composition, containing the compounds of the general formula (I) as defined above, their derivatives, their analogs, their tautomeric forms, their stereoisomers, their polymorphs, their pharmaceutically acceptable salts or their pharmaceutically acceptable solvates in combination with the usual pharmaceutically employed carriers, diluents and the like are useful for the treatment and/or prophylaxis of diseases such as hypertension, coronary heart disease, atherosclerosis, stroke, peripheral vascular diseases and related disorders. These compounds are useful for the treatment of hyperlipemeia, hyperglycemia, familial hypercholesterolemia, hypertriglyceridemia, lowering of atherogenic lipoproteins, VLDL and LDL. The compounds of the present invention can be used for the treatment of certain renal diseases including glomenrulonephritis, glomerulosclerosis, nephrotic syndrome, hypertensive nephrosclerosis, nephropathy. The compounds of general formula (I) are also useful for the treatment/prophylaxis of insulin resistance (type II diabetes), leptin resistance, impaired glucose tolerance, dyslipidemia, disorders related to syndrome X such as hypertension, obesity, insulin resistance, coronary heart disease, and other cardiovascular disorders. These compounds may also be useful as aldose reductase inhibitors, for improving cognitive functions in dementia, treating diabetic complications, disorders related to endothelial cell activation, psoriasis, polycystic ovarian syndrome (PCOS), inflammatory bowel diseases, osteoporosis, myotonic dystrophy, pancreatitis, retinopathy, arteriosclerosis, xanthoma, inflammation and for the treatment of cancer. The compounds of the present invention are also useful in the treatment and/or prophylaxis of the above said diseases in combination/concomittant with one or more HMG CoA reductase inhibitors, hypolipidemic/hypolipoproteinemic agents such as fibric acid derivatives, nicotinic acid, cholestyramine, colestipol, probucol or their combination. The compounds of the present invention in combination with HMG CoA reductase inhibitors, hypolipidemic/hypolipoproteinemic agents can be administered together or within such a period to act synergistically. The HMG CoA reductase inhibitors may be selected from those used for the treatment or prevention of hyperlipidemia such as lovastatin, pravastatin, simvastatin, fluvastatin, atorvastatin, cerivastatin and their analogs thereof. Suitable fibric acid derivative may be gemfibrozil, clofibrate, fenofibrate, ciprofibrate, benzafibrate and their analogs thereof.

The present invention also provides a pharmaceutical composition, containing the compounds of the general formula (I) as defined above, their derivatives, their analogs, their tautomeric forms, their stereoisomers, their polymorphs, their pharmaceutically acceptable salts or their pharmaceutically acceptable solvates and one or more HMG CoA reductase inhibitors, hypolipidemic/hypolipoproteinemic agents such as fibric acid derivatives, nicotinic acid, cholestyramine, colestipol, probucol in combination with the usual pharmaceutically employed carriers, diluents and the like.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. Such compositions typically contain from 1 to 20%, preferably 1 to 10% by weight of active compound, the remainder of the composition being pharmaceutically acceptable carriers, diluents or solvents.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the compound of formula (I) can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the compound formula (I) can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the compound of formula (I), of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder or the like are particularly suitable for any oral application. Preferably, carriers for tablets, dragees or capsules include lactose, corn starch and/or potato starch. A syrup or elixir can be used in cases where a sweetened vehicle can be employed.

A typical tablet production method is exemplified below:

Tablet Production Example

| | | |
|---|---|---|
| a) | 1) Active ingredient | 30 g |
| | 2) Lactose | 95 g |
| | 3) Corn starch | 30 g |
| | 4) Carboxymethyl cellulose | 44 g |
| | 5) Magnesium stearate | 1 g |
| | | 200 g for 1000 tablets |

The ingredients 1 to 3 are uniformly blended with water and granulated after drying under reduced pressure. The ingredient 4 and 5 are mixed well with the granules and compressed by a tabletting machine to prepare 1000 tablets each containing 30 mg of active ingredient.

| | | |
|---|---|---|
| b) | 1) Active ingredient | 30 g |
| | 2) Calcium phosphate | 90 g |
| | 3) Lactose | 40 g |
| | 4) Corn starch | 35 g |
| | 5) Polyvinyl pyrrolidone | 3.5 g |
| | 6) Magnesium stearate | 1.5 g |
| | | 200 g for 1000 tablets |

The ingredients 1-4 are uniformly moistened with an aqueous solution of 5 and granulated after drying under reduced pressure. Ingredient 6 is added and granules are compressed by a tabletting machine to prepare 1000 tablets containing 30 mg of ingredient 1.

The compound of the formula (I) as defined above are clinically administered to mammals, including man, via either oral, nasal, pulmonary, transdermal or parenteral, rectal, depot, subcutaneous, intravenous, intraurethral, intramuscular, intranasal, ophthalmic solution or an ointment. Administration by the oral route is preferred, being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range of about 0.01 to about 100 mg/kg body weight of the subject per day or preferably about 0.01 to about 30 mg/kg body weight per day administered singly or as a divided dose. However, the optimum dosage for the individual subject being treated will be determined by the person responsible for treatment, generally smaller doses being administered initially and thereafter increments made to determine the most suitable dosage.

The invention is explained in detail in the examples given below which are provided by way of illustration only and therefore should not be construed to limit the scope of the invention.

Preparation 1

### Ethyl 2-bromopentanoate

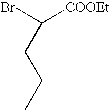

Red phosphorous (455 mg, 14.7 mmol) was added to valeric acid (15 g, 14.7 mmol) followed by drop wise addition of bromine (15 mL, 294 mmol) at room temperature. After complete addition, the reaction mixture was heated to 100° C. for 6 h. The excess bromine was then removed using water aspirator at the same temperature. Ethanol (25 mL) was added to the residue and refluxed for overnight. The reaction mixture was cooled to room temperature and poured into water (45 mL) when oil separated. The oil was washed with water, saturated sodium bicarbonate and water, dried (Na₂SO₄) and distilled under reduced pressure to obtain the title compound (26.2 g, 85%) as colorless oil.

¹H NMR (CDCl₃): δ 0.94 (t, J=7.3 Hz, 3H), 1.29 (t, 3=7.1 Hz, 3H), 1.35-1.56 (m, 2H), 1.90-2.13 (m, 2H), 4.15-4.29 (m, 3H).

Preparation 2

### Ethyl 2-(4-aminophenylsulfanyl)pentanoate

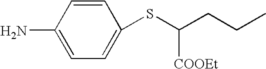

To a cooled solution of 4-aminothiophenol (5.40 g, 43.2 mmol) in N,N-dimethylformamide (30 mL) sodium hydride (60% oil coated, 1.9 g, 47.5 mmol) was added portion wise. After stirring at room temperature for 30 min ethyl 2-bromopentanoate (14.44 g, 69.1 mmol), obtained in preparation 1, in N,N-dimethylformamide (25 mL) was added to the above reaction mixture at 0° C. with vigorous stirring. The stirring was continued at room temperature for further 48 h. The reaction mixture was diluted with ethyl acetate (400 mL) and washed the organic layer successively with water (300 mL) and brine, dried over anhydrous sodium sulfate and concentrated. The residue was purified by column chromatography using 10% ethyl acetate in pet ether to afford the title compound (6.04 g, 55.3%) as yellow oil.

¹H NMR (CDCl₃): δ 0.90 (t, J=7.0 Hz, 311), 1.24 (t, J=7.1 Hz, 3H), 1.30-1.55-1.91 (m, 2H), 3.42 (dd, J=8.1 and 6.6 Hz, 1H), 4.09 (q, J=7.1 Hz, 2H) 8.8 Hz, 2H), 7.26 (d, 3=8.3 Hz, 2H).

Preparation 3

### Ethyl 2-(4-heptylaminophenylsulfanyl)pentanoate

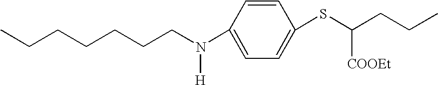

The title compound (586 mg, 14.25%) was obtained as light brown colored liquid from ethyl 2-(4-amionophenylsulfanyl)pentanoate (2.96 g, 11.7 mmol) obtained in preparation 2, heptyl bromide (2 mL, 12.9 mmol) and sodium hydride (60% oil coated, 516 mg, 12.9 mmol) by following the similar procedure as described in preparation 2.

¹H NMR (CDCl₃): δ 0.92-1.00 (m, 6H), 1.21 (t, J=7.1 Hz, 3H), 1.30-1.88 (m, 14H), 3.10 (t, 3=7.1 Hz, 2H), 3.41 (dd, J=8.3 and 6.8 Hz, 1H), 4.10 (q, J=7.2 Hz, 2H), 6.52 (d, J=8.8 Hz, 2H), 7.29 (d, J=8.3 Hz, 2H).

Preparation 4

### Ethyl 2-[4-chloromethyl(heptyl)carboxamidophenylsulfanyl]pentanoate

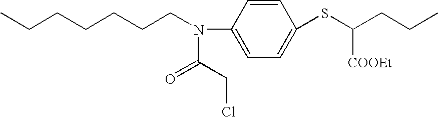

To a cooled solution of ethyl 2-(4-heptylaminophenylsulfanyl)pentanoate (586 mg, 1.66 mmol), obtained in preparation 3, in dichloromethane (10 mL) triethyl amine (0.58 mL, 4.17 mmol) was added drop wise. After stirring at room temperature for 15 min chloroacetyl chloride (0.3 mL, 3.77 mmol) was added at 0° C. and the stirring was continued at room temperature for further 12 h. The reaction mixture was diluted with dichloromethane (10 mL) and washed the organic layer successively with water (10 mL) and brine, dried over anhydrous sodium sulfate and concentrated. The residue was purified by column chromatography using 8% ethyl acetate in pet ether to afford the title compound (597 mg, 84%) as oil.

¹H NMR (CDCl₃): δ 0.88 (t, J=6.1 Hz, 3H), 0.98 (t, J=7.3 Hz, 3H), 1.15-1.60 (m, 15H), 1.71-2.03 (m, 2H), 3.65-3.88 (m, 5H), 4.17 (q, J=7.2 Hz, 2H), 7.17 (d, J=8.3 Hz, 2H), 7.52 (d, J=8.3 Hz, 2H).

Preparation 5

### Ethyl(2S)-3-[4-(2-bromoethoxy)phenyl]-2-ethoxypropanoate

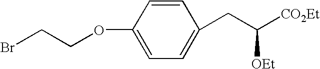 Step-i:

4-Hydroxybenzaldehyde (100 g), potassium carbonate (226 g) and dimethylformamide (500 ml) were taken in a reaction flask and stirred for 15 minutes. Benzylchloride (114 g) was then added at room temperature and the reaction mass was stirred at the same temperature for a period of 8-10 h. The progress of the reaction was monitored with TLC. After completion of the reaction, the reaction mixture was dumped into ice water and extracted with toluene. The combined organic extracts were washed with 10% aq. sodium hydroxide solution followed by water. The solvent was removed under reduced pressure and the resulting residue was triturated with pet. ether to afford 4-benzyloxybenzaldehyde (160-165 g, 93-95%).

Step-ii:

4-Benzyloxybenzaldehyde obtained in step (i) above, was added to a solution of sodium ethoxide (64 g) in absolute ethanol followed by the addition of ethylchloroacetate (87 g) over a period of 30-45 minutes, maintaining ambient temperature. The reaction mixture was stirred at the same temperature for 3-5 h. The progress of the reaction was monitored with TLC. After completion of the reaction, the reaction mixture was dumped into ice water and stirred for 5-10 minutes. The solid thus obtained was filtered, washed with water and dried to afford ethyl-2,3-epoxy-3-(4-benzyloxyphenyl)propionate (126-129 g, 90-92%).

Step-iii:

A mixture of ethyl-2,3-epoxy-3-(4-benzyloxyphenyl)propionate (125 g) obtained in step (ii) above, 5% Pd/C catalyst (12.5 g) and 1,4-dioxane (750 ml) was hydrogenated at room temperature at 5-10 psi of hydrogen pressure for a period of 10-15 h. The progress of the reaction was monitored by TLC. After completion of the reaction, the catalyst was filtered and the solvent was removed under reduced pressure to afford racemic ethyl 2-hydroxy-3-(4-benzyloxyphenyl)propionate (100-105 g, 80-83%).

Step-iv:

A mixture of racemic ethyl 2-hydroxy-3-(4-benzyloxyphenyl)propionate (100 g) obtained in step (iii) above and 10% aq. sodium hydroxide solution (500 ml) was stirred at room temperature for a period of 1-2 h. The progress of the reaction was monitored by TLC. After completion of the reaction, the reaction mixture was acidified with dil. hydrochloric acid at 15-20° C. and the solid thus obtained was filtered and washed with water to afford racemic 2-hydroxy-3-(4-benzyloxyphenyl)propionic acid (72-77 g, 80-85%).

To a solution of racemic 2-hydroxy-3-(4-benzyloxyphenyl)propionic acid (75 g) obtained according to the procedure described above in ethylacetate (1.1 L) was added R(+)-α-methylbenzylamine (33 g) and the mixture was stirred at room temperature for a period of 2-4 h. The solid thus obtained was filtered and the acid was regenerated after acidification to afford S(−)-2-hydroxy-3-(4-benzyloxyphenyl)propionic acid of the formula (11), (33-35 g, 44-47%). The R(+)-α-hydroxy acid obtained from the mother liquor was racemised and mixed with subsequent batches for resolution. The overall yield obtained by several such reprocesses was 90-95%.

Step-v:

To a slurry of sodium hydride (60% suspension in oil, 13 g) and dimethyl formamide (70 ml) was added a solution of S(−)-2-hydroxy-3-(4-benzyloxyphenyl)propionic acid (35 g) obtained in step (iv) above, in dimethylformamide (105 ml) at 5-10° C. over a period of 15-30 minutes. The temperature was allowed to attain room temperature and maintained at the same temperature for 1-3 hours. Ethyl iodide (62 g) was added slowly by maintaining the temperature at 25-30° C. over a period of 2-3 hours. The progress of the reaction was monitored by TLC. After completion of the reaction, the reaction mixture was dumped into ice water and extracted with toluene. The combined organic extracts were washed with water and the solvent was removed under reduced pressure to afford S(−)-ethyl 2-ethoxy-3-(4-benzyloxyphenyl)propionate (41-42 g, 98-99%).

Step-vi:

A mixture of S(−)-ethyl 2-ethoxy-3-4-benzyloxyphenyl)propionate (40 g) obtained in step (v) above, 5% Pd/C catalyst (8 g) and tetrahydrofuran (120 ml) was subjected to hydrogenation at room temperature and 50-60 psi of hydrogen pressure for a period of 8-12 hours. After completion of the reaction, the catalyst was filtered off and the solvent was removed under reduced pressure to afford S(−)-ethyl 2-ethoxy-3-(4-hydroxyphenyl)propionate (28-29 g, 97-98%).

Step-vii:

Ethyl(2S)-3-(4-hydroxyphenyl)-2-ethoxypropanoate obtained in step vi above (18 g, 75.63 mmol) and anhydrous K₂CO₃ (31.14 g, 225.65 mmol) were brought to reflux temperature in dry acetone (180 mL). 1,2-Dibromoethane (32.58 mL, 377.45 mmol) was added to the reaction mixture at reflux temperature and refluxed for 48 h. Solid K₂CO₃ was filtered off and the filtrate was concentrated under vacuum. The residue was dissolved in ethyl acetate and washed with water, dried (Na₂SO₄) and concentrated. The residue was chromatographed on silica gel using 10% ethyl acetate in pet ether to yield the title compound (12.5 g, 47.9%) as liquid and the starting material was recovered (6 g), mp 170-173° C.

[α]_{D}²⁵=−14.0° (c=1.0, CH₃OH).

¹H NMR (CDCl₃): δ 1.18 (t, J=6.8 Hz, 3H), 1.25 (t, 3=6.8 Hz, 3H), 2.96 (d, J=6.8 Hz, 2H), 3.28-3.47 (m, 1H), 3.51-3.88 (m, 3H), 3.98 (t, J=6.6 HZ, 1H), 4.18 (q, J=7.1 Hz, 2H), 4.28 (t, J=6.3 Hz, 2H), 6.84 (d, J=8.8 Hz, 2H), 7.1 (d, J=8.3 Hz, 2H).

Preparation 6

### 2-Ethoxy-3-[4-(2-hydroxyethoxy)phenyl]propanoic Acid

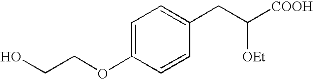 Step i:

A solution of triethyl-2-ethoxyphosphonoacetate prepared (5.61 g, 20.89 mmol) in dry tetrahydrofuran (20 mL) was added slowly to a stirred ice cooled suspension of sodium hydride (60% dispersion of oil) (1 g, 42 mmol) in dry tetrahydrofuran (10 mL), under a nitrogen atmosphere. The mixture was stirred at 0° C. for 30 min. prior to the addition of a 4-(2-bromoethoxy)benzaldehyde (4.0 g, 17.4 mmol) in dry tetrahydrofuran (30 mL). The mixture was allowed to warm up to room temperature and stirred at that temperature for further 20 h. The solvent was evaporated, water (100 mL) was added and extracted with ethyl acetate (2×75 mL). The combined organic extracts were washed with water (50 mL), brine (50 mL), dried (Na₂SO₄), filtered and the solvent was evaporated under reduced pressure. The residue was chromatographed over silica gel using a mixture of ethyl acetate and pet. ether (2:8) as an eluent to afford ethyl(E/Z)-3-[4-2-bromoethoxy)phenyl]-2-ethoxypropenoate (4.0 g, 66%) as an oil in 45:55 ratio of E:Z isomers (as measured by ¹H NMR).

¹H NMR (CDCl₃, 200 MHz): δ 1.17 and 1.42 (6H, E and Z triplets, isomeric OCH₂CH₃ and OCH₂CH₃), 3.62-3.72 (complex, 2H), 3.90-4.28 (complex, 2H), 4.30-4.37 (complex, 4H), 6.09 (s, 0.45H, olefinic proton of E isomers), 6.85 and 6.92 (2H, d and d, J=8.67 Hz and 8.7 Hz), 6.98 (s, 0.55H, Z isomer of olefinic proton), 7.16 and 7.78 (d and d, combined 2H, J=8.63 Hz and 8.72 Hz).

Step ii.

Ethyl(E/Z)-3-[4-(2-bromoethoxy)phenyl]-2-ethoxypropenoate obtained in step i. above (5.0 g, 14.5 mmol) and H₂/10% Pd—C (4 g) in dioxane (50 ml) was stirred at 25° C. under 60 psi hydrogen pressure for 24 h. The catalyst was filtered and the solvent was evaporated under reduced pressure. The residue was chromatographed over silica gel using a mixture of ethyl acetate and pet. ether (2:8) as an eluent to afford ethyl 3-[4-(2-bromoethoxy)phenyl]-2-ethoxypropanoate (4.0 g, 80%) as a colorless oil.

¹H NMR (CDCl₃, 200 MHz): δ 1.12-1.36 (complex, 6H), 2.95 (d, J=6.64 Hz, 2H), 3.25-3.45 (complex, 1H), 3.56-3.68 (complex, 3H), 3.96 (t, J=6.65 Hz, 1H), 4.16 (q, J=7.1 Hz, 2H), 4.27 (t, J=6.3 Hz, 2H), 6.81 (d, J=8.67 Hz, 2H), 7.16 (d, J=8.63 Hz, 2H).

Step iii.

Method A: Ethyl 3-[4-(2-bromoethoxy)phenyl]-2-ethoxypropanoate obtained in step ii. above (1.7 g, 4.93 mmol) was taken in potassium hydroxide solution (1.1 g, 19.71 mmol in 18 mL of water) and stirred at room temperature for 24 h. Then the reaction mixture was heated to 80-85° C. for 4-5 days. The reaction mixture was cooled to 0° C. and acidified with 2N HCl solution to pH ˜2 and extracted with ethyl acetate (3×50 mL). The combined organic extracts were washed with brine, dried (Na₂SO₄) and evaporated to dryness. The residue was purified by column chromatography on silica gel using 25% pet ether in ethyl acetate to afford the title compound (0.93 g, 74%).

Method B: To a solution of ethyl 3-[4-(2-bromoethoxy)phenyl]-2-ethoxypropanoate obtained in step ii. above (2.5 g, 7.25 mmol) in methanol (40 mL) was added a solution of sodium carbonate (3.84 g, 36.2 mmol) in water (20 mL), and stirred the reaction mixture for 24 h at room temperature. Methanol was removed under reduced pressure and the residue was diluted with water (50 mL) and extracted with ethyl acetate (2×50 mL) to remove impurities, if any. The aqueous layer was cooled to 0° C. and acidified with 2N HCl solution to pH-2 and extracted with ethyl acetate (2×200 mL). The organic extracts were washed with brine, dried (Na₂SO₄) and evaporated to dryness to give the crude intermediate (2.2 g, 96%).

The crude residue (2.2 g) was dissolved in potassium hydroxide solution (1.56 g in 20 mL of water) and the reaction mixture was stirred at room temperature for 96 h. The reaction mixture was cooled to 0° C. and acidified with 2N HCl solution to pH ˜2 and extracted with ethyl acetate (3×100 mL). The combined organic extracts were washed with brine, dried (Na₂SO₄) and evaporated to dryness. The residue was purified by column chromatography on silica gel using 25% pet ether in ethyl acetate to afford the title compound (1.5 g, 81%).

¹H NMR (CDCl₃): δ 1.19 (t, J=7.1 Hz, 3H), 2.88-3.18 (m, 2H), 3.39-3.70 (m, 2H), 3.93-4.25 (m, 5H), 6.86 (d, J=8.8 Hz, 2H), 7.17 (d, J=8.8 Hz, 2H).

Preparation 7

### Ethyl 2-ethoxy-3-[4-(2-hydroxyethoxy)phenyl]propanoate

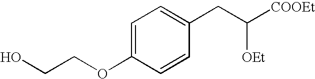

To a cooled solution of 2-ethoxy-3-[4-(2-hydroxyethoxy)phenyl]propanoic acid obtained in preparation 6 (0.92 g, 3.62 mmol) in ethanol (10 mL), conc. H₂SO₄ (1 mL) was added slowly and the reaction mixture was refluxed for 6 h. Ethanol was removed under reduced pressure. The residue was taken in ethyl acetate (100 mL), washed with saturated sodium bicarbonate solution, water, brine and dried over Na₂SO₄, and concentrated. The residue was purified on silica gel column using 10% ethyl acetate in pet ether to afford the title compound (0.72 g, 70.5%).

¹H NMR (CDCl₃): δ 1.16 (t, J=6.8 Hz, 3H), 1.22 (t, J=6.8 Hz, 3H), 2.95 (d, J=6.3 Hz, 2H), 3.27-3.42 (m, 1H), 3.51-3.70 (m, 1H), 3.92-4.08 (m, 5H), 4.16 (q, J=7.2 Hz, 2H), 6.83 (d, J=8.3 Hz, 2H), 7.16 (d, J=8.8 Hz, 2H).

Preparation 8

### (2S)-2-Ethoxy-3-[4-(2-hydroxyethoxy)phenyl]propanoic Acid

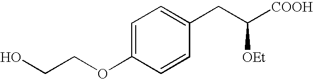

The title compound (0.72 g, 49%) was prepared from ethyl(2S)-3-[4-(2-bromoethoxy)phenyl]-2-ethoxypropanoate obtained in preparation 5 (2 g, 5.8 mmol) by following similar procedure as described in preparation 6, method B.

¹H NMR (CDCl₃): δ 1.17 (t, J=7.1 Hz, 3H), 2.90-3.15 (m, 2H), 3.39-3.70 (m, 2H), 3.95 (t, J=4.1 Hz, 21), 3.99-4.19 (m, 3H), 6.84 (d, J=8.3 Hz, 2H), 7.16 (d, J=8.8 Hz, 2H).

Preparation 9

### Ethyl(2S)-2-ethoxy-3-[4-(2-hydroxyethoxy)phenyl]propanoate

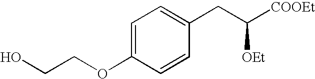

The title compound (0.37 g, 48%) was prepared from (2S)-2-ethoxy-3-[4-(2-hydroxyethoxy)phenyl]propanoic acid obtained in preparation 8 (0.7 g, 2.76 mmol) by following the similar procedure as described in preparation 7.

[α]_{D}²⁵=−17.8° (c=0.5, CHCl₃).

¹H NMR (CDCl₃): δ 1.16 (t, J=6.8 Hz, 3H), 1.23 (t, J=7.3 Hz, 3H), 2.95 (d, J=6.4 Hz, 2H), 3.26-3.43 (m, 1H), 3.51-3.69 (m, 1H), 3.92-4.10 (m, 51), 4.17 (q, J=7.2 Hz, 2H), 6.84 (d, J=8.3 Hz, 2H), 7.17 (d, J=8.3 Hz, 2H).

Preparation 10

### 4-Aminobenzoyl-1-methyl-3-propyl-1H-pyrazole-5-carboxamide

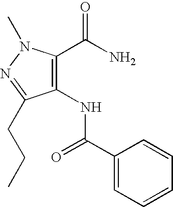 Step i.

Sodium (11.5 g, 500 mmol) was added to ethanol (130 mL) in portions at room temperature with vigorous stirring. After sodium got dissolved, the solution was cooled to 0° C. Methyl propyl ketone (53.0 mL, 500 mmol) was added dropwise and the stirring was continued for further 15-20 min. Diethyl oxalate (68.0 mL, 500 mmol) was added dropwise to the resulting solution at 0° C. and the stirring was continued for another 15 min at 0° C. The reaction mixture was then allowed to attain room temperature and the stirring was continued for further 12 h at this temperature. The reaction mixture was then kept in refrigerator for 24 h. The solvent was removed under vacuum at room temperature. The resulting residue was diluted with dil. hydrochloric acid on an ice bath and extracted the aqueous layer with diethyl ether (4×50 mL). The combined ether extracts were washed with water, brine, dried (Na₂SO₄) and concentrated under vacuum. The crude mass was chromatographed on silica gel using 3% ethyl acetate in pet ether as eluent to yield ethyl 2,4-dioxoheptanoate (60.0 g, 64.5%).

¹H NMR (CDCl₃): δ 14.45 (broad s, D₂O exchangeable, 1H), 6.33 (s, 1H), 4.32 (q, J=7.10 Hz, 2H), 2.44 (t, J=7.40 Hz, 2H), 1.72-1.57 (m, 2H), 1.35 (t, J=7.30 Hz, 3H), 0.94 (t, J=7.40 Hz, 3H).

Step ii.

To a stirred mixture of ethyl 2,4-dioxoheptanoate (28.0 g, 150 mmol) obtained in step i. above, acetic acid (225 mL) and methoxy ethanol (225 mL), hydrazine hydrochloride (31.6 g, 300 mmol) was added and heated to 105° C. for 3 h. Acetic acid and methoxy ethanol were removed under vacuum at 90° C. The residue was taken in water and extracted three times with ethyl acetate. The combined organic layers were washed with water, brine, dried (Na₂SO₄) and concentrated to obtain solids suspended in liquid. The filtrate was evaporated to yield Ethyl 3-propyl-1H-5-pyrazole carboxylate (16.0 g, 58.4%) as liquid.

¹H NMR (2A) (CDCl₃): δ 6.57 (s, 1H), 4.34 (q, J=7.10 Hz; 2H), 2.65 (t, J=7.50 Hz, 2H), 1.71-1.59 (m, 2H), 1.33 (t, J=7.20 Hz, 3H), 0.92 (t, J=7.30 Hz, 3H).

Step ii.

Dimethyl sulfate (6.0 g, 48.35 mmol) was added to the pyrazole ester obtained in step ii. above (16.0 g, 88 mmol) and the reaction mixture was heated at 160° C. for 2 h. The reaction mixture was cooled to ˜90° C. and 5N sodium hydroxide solution (64 mL) was added and the reaction mixture was stirred at 80-90° C. for 30 min. The reaction mixture was cooled in an ice bath and was acidified to pH 4 with 2N hydrochloric acid resulting precipitation of the product. The precipitate was filtered, washed with cold water and dried under vacuum to yield 1-Methyl-3-propyl-1H-5-pyrazole carboxylic acid (12.0 g, 81%). ¹HNMR (CDCl₃): δ 6.70 (s, 1H), 4.14 (s, 3H), 2.64 (t, J=7.60 Hz, 2H), 1.72-1.61 (m, 2H), 0.95 (t, J=7.30 Hz, 3H).

Step iv.

Nitrating agent was prepared by adding 90% nitric acid (7.18 mL) to concentrated sulphuric acid (13.8 mL) at 75-78° C. 1-Methyl-3-propyl-1H-5-pyrazole carboxylic acid (11.5 g, 68.4 mmol) obtained in step iii. above was added to the nitrating agent portion wise with stirring so that the temperature is maintained at ˜85° C. After the complete addition, the mixture was heated at 100° C. for 2 h. The reaction mixture was cooled and poured into crushed ice. The suspension was filtered cold (10° C.) and the solids were washed with ice cold brine and dried to yield 1-Methyl-4-nitro-3-propyl-1H-5-pyrazole carboxylic acid (5.0 g, 34%).

¹H NMR (CDCl₃): δ 6.90 (broad s, D₂O exchangeable, 1H), 4.20 (s, 3H), 2.90 (t, J=7.60 Hz, 2H), 1.79-1.64 (m, 2H), 1.02 (t, J=7.40 Hz, 3H).

Step v.

Thionyl chloride (7.2 mL, 98 mmol) was added to 1-methyl-4-nitro-3-propyl-1H-5-pyrazole carboxylic acid obtained in step iv. (3.3 g, 15.5 mmol) and refluxed for 3.5 h. The excess thionyl chloride was removed from the reaction mixture under vacuum and the residue was taken in dry acetone (20 mL). Ammonia gas was passed through this solution till pH reached 8-9. The precipitate formed was filtered. The filtrate was concentrated and then dissolved in ethyl acetate. The organic phase was washed with water, brine, dried (Na₂SO₄) and concentrated to yield 1-Methyl-4-nitro-3-propyl-1H-5-pyrazole carboxamide as a fluffy material (3.1 g, 94%).

¹H NMR (CDCl₃): δ 7.50 (broad s, D₂O exchangeable, 1H), 6.08 (broad s, D₂O exchangeable, 1H), 4.05 (s, 31), 2.86 (t, J=7.70 Hz, 2H), 1.75-1.60 (m, 2H), 0.98 (t, J=7.30 Hz, 3H).

Step vi.

To a solution of 1-methyl-4-nitro-3-propyl-1H-5-pyrazole carboxamide obtained in step v. (3.2 g, 15.0 mmol) in methanol (30 mL), Raney nickel (450 mg) was added and hydrogenated by passing hydrogen gas at 50 psi for 10 h. The reaction mixture was filtered through celite bed and washed with methanol. The filtrate and washings were combined, concentrated, purified by silica gel column chromatography using ethyl acetate-pet. ether (1:1) as eluent to yield 4-amino-1-methyl-3-propyl-1H-5-pyrazole carboxamide (2.0 g, 73%).

¹H NMR (CDCl₃): δ 7.05 (broad s, D₂O exchangeable, 2H), 4.07 (s, 3H), 2.75 (broad s, D₂O exchangeable, 2H), 2.51 (t, J=7.60 Hz, 2H), 1.69-1.50 (m, 2H), 0.95 (t, J=7.30 Hz, 3H).

Step vii.

To a solution of 4-amino-1-methyl-3-propyl-1H-5-pyrazole carboxamide obtained in step vi. above (550 mg, 3.0 mmol) in dichloromethane (8 mL) triethyl amine (0.92 mL, 6.6 mmol) was added at 0° C. and the reaction mixture was stirred for 15 min. Benzoyl chloride (0.39 mL, 3.32 mmol) was added drop wise at 0° C. and the stirring was continued for further 1 h at the same temperature. The reaction mixture was diluted with dichloromethane and washed successively with water and brine, dried (Na₂SO₄) and concentrated to give the title compound (680 mg, 70%) as a solid, mp 140° C.

¹H NMR (CDCl₃): δ 0.92 (t, J=7.6 Hz, 31), 1.50-1.75 (m, 2H), 2.51 (t, J=7.6 Hz, 2H), 3.95 (s, 3H), 7.35-7.65 (m, 31), 7.91 (d, 3=7.5 Hz, 2H).

Preparation 11

### 1-Methyl-5-phenyl-3-propyl-6,7-dihydro-1H-pyrazolo[4,3-d]pyrimidin-7-one

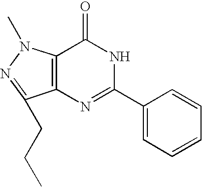

To a stirred solution of potassium tert-butoxide (440 mg, 4.4 mmol) in t-butanol (10 mL) 4-aminobenzoyl-1-methyl-3-propyl-1H-pyrazole-5-carboxamide (1.0 g, 3.67 mmol), obtained in the preparation 10, was added portion wise and the reaction mixture was refluxed for 6 h. The solvent was removed under vacuum and the resulting residue was diluted with water and acidified with dil. HCl to pH˜5 at 0° C. The resulting white precipitate was filtered, washed with water and dried to afford the title compound (700 mg, 74.7%).

¹H NMR (CDCl₃): δ 1.02 (t, J=7.3 Hz, 311), 1.79-2.10 (m, 2H), 2.93 (t, J=7.5 Hz, 2H), 4.27 (s, 3H), 7.5.1 (br s, 3H), 8.10-8.17 (m, 2H).

Preparation 12

### 1-Methyl-3-propyl-5-cyclopropyl-1H-pyrazolo[4,3-d]pyrimidin-7-one

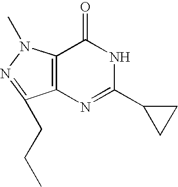

Cyclopropane carboxylic acid (600 mg, 6.97 mmol) was refluxed in thionyl chloride (1.01 mL, 1.64 mmol) for 2 h. Excess thionyl chloride was distilled off and the acid chloride was cooled to 0° C. In another set up, to a solution of 4-amino-1-methyl-3-propyl-1H-5-pyrazole carboxamide (prepared as disclosed in our U.S. application Ser. No. 09/507,373) (1.0 g, 5.49 mmol) in xylene (10 mL), cyclopropane carboxylic acid (5 mL) was added and cooled in ice bath. Triethyl amine was added to the above mixture and stirred for 20 min. This mixture was then added to acid chloride at 0° C. and stirred at room temperature for 1 h. The reaction mixture was refluxed for 48 h and the solvents were removed under reduced pressure. The residue was diluted with water and extracted with ethyl acetate (3×60 mL). The combined organic layers were washed with water, brine, dried (Na₂SO₄) and concentrated. The crude compound was purified by silica gel column chromatography using 30% ethyl acetate in pet ether to yield the title compound (1.22 g, 45%) as a white solid, mp 228-230° C.

¹H NMR (CDCl₃): δ 0.98 (t, J=7.3 Hz, 3H), 1.01-1.25 (m, 4H), 1.72-1.99 (m, 3H), 2.82 (t, J=7.6 Hz, 21), 4.23 (s, 3H), 11.05 (br s, 1H, D₂O exchangeable).

Preparation 13

### Ethyl 2-(4-nitrophenoxy)pentanoate

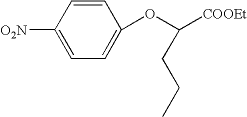

To a solution of 4-nitrophenol (11.08 g, 79.7 mmol) in N,N-dimethylformamide (70 mL) anhydrous K₂CO₃ (28.1 g, 203.8 mmol) was added and stirred for 30 min at room temperature. Ethyl 2-bromopentanoate obtained in preparation 1 (20.0 g, 95.7 mmol) in N,N-dimethylformamide (25 mL) was added to the above reaction mixture with vigorous stirring. The stirring was continued at room temperature for further 18 h. The reaction mixture was diluted with ethyl acetate (400 mL) and washed the organic layer successively with water (3×300 mL)_{y} and brine, dried over anhydrous sodium sulfate and concentrated to afford the title compound (23.0 g, 92%) as a gummy mass.

¹H NMR (CDCl₃): δ 0.99 (t, J=7.6 Hz, 3H), 1.25 (t, J=7.1 Hz, 3H), 1.42-1.64 (m, 2H), 1.92-2.04 (m, 2H), 4.22 (q, J=7.2 Hz, 2H), 4.71 (t, J=6.1 Hz, 1H), 6.92 (d, J=9.3 Hz, 2H), 8.18 (d, J=9.3 Hz, 2H).

Preparation 14

### Ethyl 2-(4-aminophenoxy)pentanoate

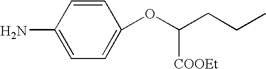

To a solution of ethyl 2-4-nitrophenoxy)pentanoate obtained in preparation 13 (22.5 g, 84.3 mmol) in dioxane (65 mL), 10% Pd—C (5.63 g) was added and hydrogenated by passing hydrogen gas at 40 psi at room temperature for 5 h. The reaction mixture was filtered through celite bed and washed with dioxane. The filtrate and washings were combined, concentrated, and purified by silica gel column chromatography using 20% ethyl acetate in pet ether as eluent to yield the title compound (14.0 g, 70%).

¹H NMR (CDCl₃): δ 0.94 (t, J=7.1 Hz, 3H), 1.22 (t, J=7.1 Hz, 3H), 1.40-1.62 (m, 2H), 1.78-1.96 (m, 2H), 4.17 (q, J=7.1 Hz, 2H), 4.45 (t, J=6.3 Hz, 1H), 6.5 (d, J=6.8 Hz, 2H), 6.73 (d, J=6.3 Hz, 2H).

Preparation 15

### Ethyl 6-[4-(1-ethoxycarbonylbutoxy)amino]hexanoate

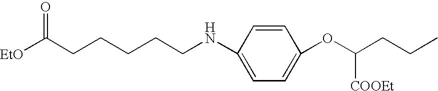

The title compound (1.55 g, 48.5%) was obtained as a liquid from ethyl 2-4-aminophenoxy)pentanoate (2.0 g, 8.4 mmol) obtained in preparation 14 by reacting with ethyl 6-bromohexanoate (2.26 g, 10.1 mmol) using anhydrous potassium carbonate (3.5 g, 25.31 mmol) at room temperature for 48 h by following the similar procedure as described in preparation 13.

¹H NMR (CDCl₃): δ 0.97 (t, J=7.3 Hz, 3H), 1.25 (t, J=7.3 Hz, 611), 1.36-1.76 (m, 8H), 1.80-1.96 (m, 2H), 2.31 (t, I=7.3 Hz, 2H), 3.06 (t, I=6.8 Hz, 2H), 4.07-4.26 (m, 4H), 4.46 (t, J=6.4 Hz, 1H), 6.52 (d, J=6.8 Hz, 2H), 6.77 (d, J=6.3 Hz, 2H).

Preparation 16

### Ethyl 6-chloromethyl[4-(1-ethoxycarbonylbutoxy)phenyl]carboxamido-hexanoate

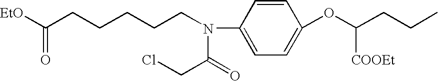

The title compound (1.45 g, 78%) was obtained from ethyl 6-[4-(1-ethoxycarbonylbutoxy)anilino]hexanoate (1.55 g, 4.1 mmol) obtained in preparation 15 by reacting with chloroacetyl chloride (0.47 mL, 6.13 mmol) using dichloromethane (18 mL) in presence of triethyl amine (1.7 mL, 12.27 mmol) at room temperature for 16 h by following the similar procedure as described in preparation 4.

¹H NMR (CDCl₃): δ 1.00 (t, J=7.3 Hz, 3H), 1.24 (t, J=7.1 Hz, 3H), 1.25-1.41 (m, 3H), 1.40-1.70 (m, 8H), 1.86-2.09 (m, 2H), 2.27 (t, J=7.6 Hz, 2H), 3.66 (t, J=7.3 Hz, 2H), 3.78 (s, 2H), 4.11 (q, J=7.1 Hz, 2H), 4.25 (q, J=6.8 Hz, 2H), 4.61 (t, J=6.8 Hz, 1H), 6.90 (d, J=8.8 Hz, 2H), 7.10 (d, J=8.8 Hz, 2H).

Preparation 17

### Ethyl 2-ethoxy-3-[4-nitrophenyl]propeonate

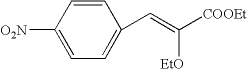

To an ice-cold slurry of sodium hydride (60% oil coated, 5.3 g, 132.5 mmol), a solution of the Wittig salt (triethyl 2-ethoxyphosphonoacetate) (23.16 g, 86.1 mmol), (prepared as disclosed in our U.S. Pat. No. 6,054,453), in THF (150 mL) was slowly added drop wise with vigorous stirring. The reaction temperature was brought to room temperature and heated until the reaction mixture became clear. The reaction mixture was cooled to 0° C. and a solution of p-nitrobenzaldehyde (10.0 g, 66.2 mmol) in THF (150 mL) was added drop wise and stirred at room temperature for overnight THF was then removed and the residue was taken in ethyl acetate, and washed successively with water and brine, dried over anhydrous sodium sulfate and concentrated. The residue was chromatographed using 5% ethyl acetate in pet ether to afford the title compound as E and Z isomers (15.1 g, 86%) as bright yellow liquid.

Z isomer. ¹H NMR (CDCl₃): δ 1.16 (t, J=7.2 Hz, 3H), 1.46 (t, J=7.0 Hz, 3H), 3.99 (q, J=7.0 Hz, 2H), 4.19 (q, J=7.1 Hz, 21), 6.04 (s, 1H), 7.34 (d, J=8.7 Hz, 2H), 8.15 (d, J=8.6 Hz, 2H).

E isomer. ¹H NMR (CDCl₃): δ 1.35-1.47 (m, 6H), 4.12 (q, J=7.0 Hz, 2H), 4.34 (q, J=7.2 Hz, 2H), 6.92 (s, 1H), 7.92 (d, J=8.8 Hz, 2H), 8.22 (d, J=8.7 Hz, 2H).

Preparation 18

### Ethyl 2-ethoxy-3-[4-aminophenyl]propanoate

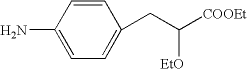

The title compound (3.2 g, 55%) was synthesized by hydrogenating ethyl 2-ethoxy-3-[4-nitrophenyl]propeonate obtained in preparation 17 (6.5 g, 24.4 mmol) by passing hydrogen gas at 60 psi, taken in dioxane (65 mL), in presence of 10% Pd—C (2.6 g) at room temperature for 36 h by following the same procedure as described in preparation 14.

¹H NMR (CDCl₃): δ 1.17 (t, J=6.1 Hz, 3H), 1.23 (t, J=6.1 Hz, 3H), 2.91 (d, J=6.7 Hz, 2H), 3.28-3.44 (m, 1H), 3.52-3.68 (m, 1H), 3.95 (t, 3=6.8 Hz, 1H), 4.17 (q, J=7.1 Hz, 2H), 6.62 (d, J=8.1 Hz, 2H), 7.04 (d, J=8.2 Hz, 2H).

Preparation 19

### Ethyl 2-ethoxy-3-[4-(4-fluorobenzylamino)phenyl]propanoate

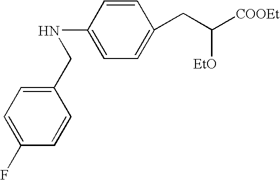

The title compound (100 mg, 14%) was obtained as yellow colored liquid from ethyl 2-ethoxy-3-[4-aminophenyl]propanoate (500 mg, 2.11 mmol) obtained in preparation 18, 1-bromomethyl-4-fluorobenzene (438 mg, 2.32 mmol) and anhydrous potassium carbonate (873 mg, 6.33 mmol) at room temperature for 18 h by following the similar procedure as described in preparation 13.

¹H NMR (CDCl₃): δ 1.16 (t, J=6.9 Hz, 3H), 1.21 (t, J=7.1 Hz, 3H), 2.89 (d, J=6.8 Hz, 2H), 3.28-3.43 (m, 1H), 3.50-3.66 (m, 1H), 3.94 (t, J=6.6 Hz, 1H), 4.15 (q, J=7.3 Hz, 2H), 4.27 (s, 2H), 6.54 (d, J=8.3 Hz, 2H), 6.94-7.09 (m, 4H), 7.32 (dd, J=8.3 and 5.4 Hz, 2H).

Preparation 20

### Ethyl 3-[4-chloromethyl(4-fluorobenzyl)carboxamidophenyl]-2-ethoxypropanoate

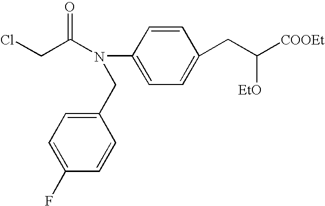

The title compound (355 mg, 74%) was obtained as straw yellow colored liquid from ethyl 2-ethoxy-3-[4-(4-fluorobenzylamino)phenyl]propanoate (390 mg, 1.13 mmol) obtained in preparation 19, chloroacetyl chloride (0.09 mL, 1.36 mmol) and dichloromethane in presence of triethyl amine (0.39 mL, 2.83 mmol) at room temperature for 16 h by following the similar procedure as described in preparation 4.

¹H NMR (CDCl₃): δ 1.14 (t, J=6.9 Hz, 3H), 1.24 (t, J=7.3 Hz, 3H), 2.95-3.06 (m, 2H), 3.25-3.42 (m, 1H), 3.55-3.70 (m, 1H), 3.83 (s, 2H), 3.94 (dd, J=7.6 and 5.6 Hz, 1H), 4.16 (q, J=7.0 Hz, 2H), 4.84 (s, 2H), 6.80-7.00 (m, 4H), 7.15 (dd, J=8.3 and 5.9 Hz, 2H), 7.25 (d, J=8.3 Hz, 2H).

Preparation 21

### Ethyl 2-[4-{4-(4-ethoxycarbonylphenyl)butylamino}phenoxy]pentanoate

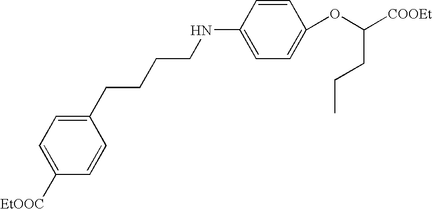

The title compound (1.35 g, 36.4%) was obtained as a liquid from ethyl 2-(4-aminophenoxy)pentanoate (2.0 g, 8.4 mmol), obtained in preparation 14, by reacting with ethyl 4-(4-bromobutyl)benzoate (2.65 g, 9.82 mmol) using anhydrous potassium carbonate (3.5 g, 25.31 mmol) at room temperature for 60 h following a similar procedure as described in the preparation 13.

¹H NMR (CDCl₃): δ 0.97 (t, J=7.3 Hz, 3H), 1.25 (t, J=7.1 Hz, 3H), 1.39 (t, J=7.3 Hz, 3H), 1.40-1.96 (m, 8H), 2.71 (t, J=7.1 Hz, 2H), 3.08 (t, J=6.6 Hz, 2H), 4.20 (t, J=7.1 Hz, 2H), 4.37 (q, J=7.5 Hz, 2H), 4.47 (t, J=7.1 Hz, 1H), 6.52 (d, J=8.8 Hz, 2H1), 6.77 (d, J=8.8 Hz, 2H), 7.25 (d, J=8.3 Hz, 2H), 7.97 (d, J=8.3 Hz, 2H).

Preparation 22

### Ethyl 2-[4-chloromethyl{4-(4-ethyloxycarbonylphenyl)butyl}carboxamido-phenoxy]pentanoate

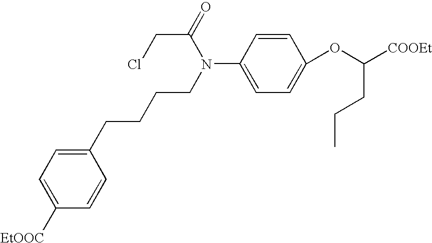

The title compound (1.4 g, 88%) was obtained from ethyl 2-[4-{4-(4-ethoxycarbonylphenyl)butylamino}phenoxy]pentanoate (1.35 g, 3.07 mmol), obtained in preparation 21, by reacting with chloroacetyl chloride (0.28 mL, 3.68 mmol) using dichloromethane (20 mL) in presence of triethyl amine (1.08 mL, 7.67 mmol) at room temperature for 40 h by following the similar procedure as described in preparation 4.

¹H NMR (CDCl₃): δ 1.00 (t, J=7.3 Hz, 311), 1.24 (t, J=7.3 Hz, 3H), 1.38 (t, J=7.3 Hz, 3H), 1.48-1.70 (m, 6H), 1.82-2.05 (m, 2H), 2.66 (t, J=6.8 Hz, 2H), 3.68 (t, J=6.8 Hz, 2H), 3.77 (s, 2H), 4.24 (t, J=7.0 Hz, 211), 4.36 (q, J=7.1 Hz, 2H), 4.60 (t, J=6.1 Hz, 1H), 6.88 (d, J=8.8 Hz, 2H), 7.05 (d, J=8.8 Hz, 2H), 7.19 (d, J=8.3 Hz, 2H), 7.93 (d, J=7.8 Hz, 2H).

Preparation 23

### 5-Ethyl-1-methyl-3-propyl-6,7-dihydro-1H-pyrazolo[4,3-]pyrimidin-7-one

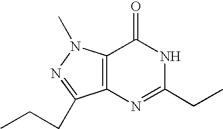

To 4-amino-1-methyl-3-propyl-1H-5-pyrazole carboxamide obtained in step vi. preparation 10 (1.3 g, 7.1 mmol) in xylene (10.5 mL), propionic acid (10.5 mL) was added and cooled in ice bath. Triethyl amine (2.2 mL, 15.7 mmol) was added to the reaction mixture and stirred for 15 min. Propionyl chloride was added and stirred at the same temperature for 30 min. The reaction mixture was refluxed for 36 h and the solvent was removed under vacuum. The resulting residue was diluted with water and extracted with ethyl acetate (3×10 mL). The combined organic extracts were washed with water, brine, dried (Na₂SO₄) and concentrated. The crude compound was purified by silica gel column chromatography using 30% ethyl acetate in pet. ether as eluent to yield the title compound (560 mg, 36%).

¹H NMR (CDCl₃): δ 11.1 (broad s, D₂O exchangeable, 1H), 4.23 (s, 3H), 2.89-2.69 (m, 4H), 1.85-1.71 (m, 2H), 1.37 (t, J=7.50 Hz, 3H), 0.98 (t, J=7.40 Hz, 3H).

Preparation 24.

### 1,5-Dimethyl-3-propyl-6,7-dihydro-1H-pyrazolo[4,3-d]pyrimidin-7-one

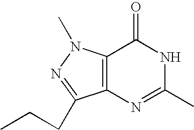

The title compound (360 mg, 36%) was obtained as fluffy solid from 4-amino-1-methyl-3-propyl-1H-5-pyrazole carboxamide (950 mg, 5.2 mmol) obtained in step vi preparation 10, acetyl chloride (450 mg, 5.74 mmol), triethyl amine (1.15 g, 11.48 mmol) and acetic acid (7.5 mL) by refluxing in xylene (7.5 mL) for 72 h following a similar procedure as described in preparation 23.

¹H NMR (CDCl₃): δ 11.18 (broad s, D₂O exchangeable, 1H), 4.23 (s, 3H), 2.84 (t, J=7.60 Hz, 2H), 2.51 (s, 3H), 1.84-1.75 (m, 2H), 0.98 (t, J=7.30 Hz, 3H).

### EXAMPLE 1

### Ethyl 2-[4-(5-ethyl-1-methyl-3-propyl-1H-pyrazolo [4,3-d]pyrimidin-7-yloxymethyl(heptyl)carboxamido)phenylsulfanyl]pentanoate

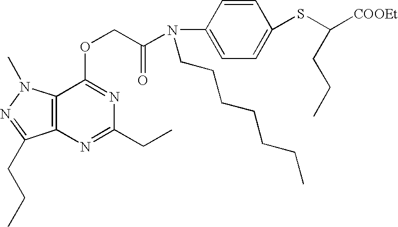

A mixture of 5-ethyl-1-methyl-3-propyl-6,7-dihydro-1H-pyrazolo[4,3-d]-pyrimidin-7-one obtained in preparation 23 (236 mg, 1.07 mmol) and anhydrous K₂CO₃ (444 mg, 3.21 mmol) in dry N,N-dimethylformamide (4 mL) was stirred under argon atmosphere for 30 mm at room temperature. Ethyl 2-[4-chloromethyl(heptyl)carboxamidophenylsulfanyl]-pentanoate (597 mg, 1.39 mmol), obtained in preparation 4, dissolved in dry N,N-dimethylformamide (3 mL) was added drop wise to the above reaction mixture at 0° C. The reaction mixture was stirred at room temperature for 48 h. The reaction mixture was diluted with ethyl acetate (25 mL), washed with water (3×15 mL), brine (20 mL), dried (Na₂SO₄) and concentrated. The residue was column chromatographed using 10% ethyl acetate in pet ether to afford the title compound (527 mg, 81%).

¹H NMR (CDCl₃): δ 0.85 (t, J=6.4 Hz, 3H), 0.96 (t J=7.6 Hz, 3H), 0.99 (t, J=7.6 Hz, 3H), 1.15-1.62 (m, 20H), 1.70-1.90 (m, 2H), 2.84-3.00 (m, 4H), 3.63-3.79 (m, 3H), 4.10-4.22 (m, 5H), 4.82 (s, 2H), 7.26 (d, 3=8.3 Hz, 2H), 7.54 (d, J=8.3 Hz, 2H).

### EXAMPLE 2

### 2-[4-(5-Ethyl-1-methyl-3-propyl-1H-pyrazolo[4,3-d]pyrimidin-7-yloxymethyl-(heptyl)carboxamido)phenylsulfanyl]pentanoic Acid

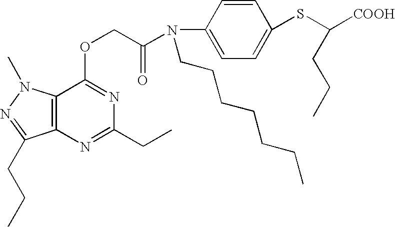

To a solution of ethyl 2-[4-(5-ethyl-1-methyl-3-propyl-1H-pyrazolo[4,3-d]pyrimidin-7-yloxymethyl(heptyl)carboxamido)phenylsulfanyl]pentanoate (527 mg, 0.86 mmol), obtained in example 1, in methanol (5 mL) sodium carbonate (457 mg, 4.31 mmol) in water (5 mL) was added and stirred at room temperature for 12 h. Methanol was removed from the reaction mixture under reduced pressure and the resulting aqueous layer was acidified with 2N HCl at 0° C. to pH-2. The aqueous layer was extracted with ethyl acetate, dried (Na₂SO₄) and concentrated. The residue was chromatographed using 60% ethyl acetate in pet ether as eluent to afford the title compound (27 mg, 5.4%), mp 84-86° C.

¹H NMR (CDCl₃): δ 0.87 (t, J=6.4 Hz, 3H), 1.01 (t, J=7.3 Hz, 6H), 1.20-1.42 (m, 11H), 1.45-1.68 (m, 3H), 1.74-1.95 (m, 21), 2.84-3.00 (m, 6H), 3.63-3.84 (m, 4H), 4.15 (s, 3H), 4.79 (s, 21), 7.27 (d, J=8.8 Hz, 2H), 7.59 (d, J=8.3 Hz, 2H).

### EXAMPLE 3

### Ethyl 2(S)-2-ethoxy-3-[4-{2-(5-ethyl-1-methyl-3-propyl-1H-pyrazolo [4,3-d]pyrimidin-7-yloxy)ethoxy}phenyl]propanoate

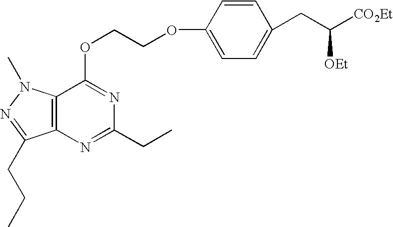

A mixture of 5-ethyl-1-methyl-3-propyl-6,7-dihydro-1H-pyrazolo-[4,3-d]pyrimidin-7-one obtained in preparation 23 (40.0 g, 180 mmol) and anhydrous K₂CO₃ (75.2 g, 544 mmol) in dry N,N-dimethylformamide (200 mL) was stirred under argon atmosphere for 30 min at room temperature. Ethyl(2S)-3-[4-(2-bromoethoxy)phenyl]-2-ethoxypropanoate (3.9 g, 11.3 mmol), obtained in preparation 5, dissolved in dry N,N-dimethylformamide (7 mL) was added drop wise to the above mixture at room temperature. The reaction mixture was stirred at room temperature for 24 h. The reaction mixture was diluted with ethyl acetate (1 L), washed with water (3×400 mL), brine (200 mL), dried (Na₂SO₄) and concentrated. The residue was column chromatographed using 10% ethyl acetate in pet ether to afford the title compound (14.36 g, 16.4%), mp 59-60° C.

[α]_{D} ²=−11.0° (c=1.0, CH₃OH).

¹H NMR (CDCl₃): δ 1.00 (t, J=7.3 Hz, 3H), 1.17 (t, I=6.6 Hz, 3H), 1.23 (t, J=6.8 Hz, 3H), 1.36 (t, J=7.3 Hz, 3H), 1.74-1.93 (m, 2H), 2.87-3.03 (m, 6H), 3.26-3.47 (m, 1H), 3.51-3.88 (m, 1H), 3.97 (t, J=6.6 Hz, 1H), 4.14 (s, 3H), 4.16 (q, J=7.3 Hz, 2H), 4.38 (t, J=4.7 Hz, 2H), 4.91 (t, J=4.7 Hz, 2H), 6.87 (d, J=8.3 Hz, 2H), 7.17 (d, J=8.3 Hz, 2H).

### EXAMPLE 4

### Methyl 2(S)-2-ethoxy-3-[4-{2-(5-ethyl-1-methyl-3-propyl-1H-pyrazolo [4,3-d]-pyrimidin-7-yloxy)ethoxy}phenyl]propanoate (A) &

### 2(S)-2-Ethoxy-3-[4-{2-(5-ethyl-1-methyl-3-propyl-1H-pyrazolo[4,3-d]pyrimidin-7-yloxy)ethoxy}phenyl]propanoic Acid (B)

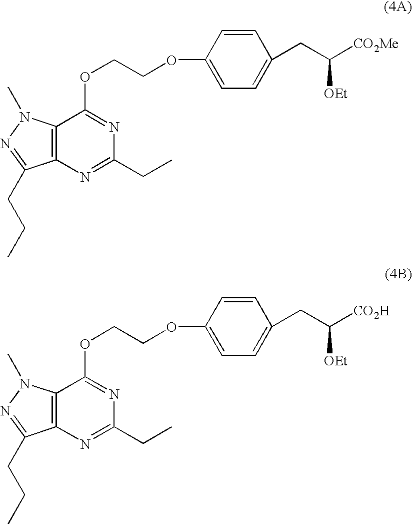

To a solution of ethyl 2(8)-2-ethoxy-3-[4-{2-5-ethyl-1-methyl-3-propyl-1H-pyrazolo[4,3-d]pyrimidin-7-yloxy)ethoxy}phenyl]propanoate (5.0 g, 10.33 mmol), obtained in example 3, in methanol (250 mL) sodium carbonate (5.47 g, 51.65 mmol) in water (25 mL) was added and stirred at room temperature for 24 h Methanol was removed from the reaction mixture under vacuum and the resulting aqueous layer was extracted with ethyl acetate which was washed with water, brine, dried (Na₇SO₄) and concentrated to get a trans esterified product i.e., the corresponding methyl ester (4A) of the free acid (1.44 g, 29.7%), mp 68-70° C. The aqueous layer was acidified with 2N HCl at low temperature to pH˜2. The solids were filtered and washed with cold water, dried under vacuum overnight to yield the title compound (4B) (2.9 g, 59.9%), mp 138-140° C.

4A: [α]_{D}²⁵=−9.0° (c=0.5, CH₃OH).

¹H NMR (CDCl₃): δ 1.01 (t, J=7.3 Hz, 3H), 1.17 (t, J=7.1 Hz, 3H), 1.23 (t, J=7.6 Hz, 3H), 1.77-1.95 (m, 2H), 2.87-3.03 (m, 6H), 3.27-3.43 (m, 1H), 3.54-3.69 (m, 1H), 3.72 (s, 3H), 4.01 (t, J=6.6 Hz, 1H), 4.16 (s, 3H), 4.40 (t, J=4.7 Hz, 2H), 4.92 (t, J=4.7 Hz, 2H), 6.88 (d, J=8.3 Hz, 2H), 7.17 (d, J=8.3 Hz, 2H).

4B: [α]_{D}²⁵=−18.6° (c=0.5, CH₃OH).

¹H NMR (CDCl₃): δ 0.99 (t, I=7.3 Hz, 311), 1.18 (t, J=6.8 Hz, 3H), 1.36 (t, J=7.6 Hz, 3H), 1.77-1.95 (m, 2H), 2.82-3.16 (m, 6H), 3.40-3.65 (m, 2H), 4.00-4.12 (m, 1H), 4.14 (s, 3H), 4.39 (t, J=4.4 Hz, 2H), 4.92 (t, J=4.7 Hz, 2H), 6.87 (d, J=8.8 Hz, 2H), 7.18 (d; J=8.8 Hz, 2H).

### EXAMPLE 5

### Ethyl 2(S)-3-[4-{2-1,5-dimethyl-3-propyl-1H-pyrazolo[4,3-dipyrimidin-7-yloxy)ethoxy}phenyl]-2-ethoxypropanoate

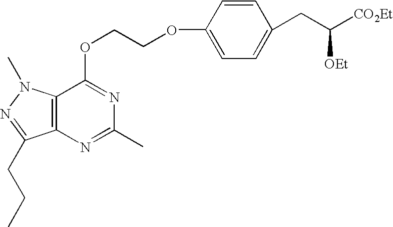

Method A: The title compound (2.26 g, 15%) was obtained as white solid by condensing 1,5-dimethyl-3-propyl-6,7-dihydro-1H-pyrazolo[4,3-d]pyrimidin-7-one (prepared as disclosed in our U.S. application Ser. No. 09/507,373) (6.6 g, 32 mmol) with ethyl(2S)-3-[4-2-bromoethoxy)phenyl]-2-ethoxypropanoate (3.9 g, 11.3 mmol), obtained in preparation 5, in dry N,N-dimethylformamide (80 mL) in the presence of anhydrous K₂CO₃ (11.05 g, 80.1 mmol) at room temperature by following the similar procedure as described in example 3, mp 84-86° C.

Method B: To a cooled solution of triphenyl phosphine (1.78 g, 6.8 mmol) in dry THF (5 mL) DIAD (1.35 mL, 6.8 mmol) was added drop wise and stirred for 15 min. 1,5-Dimethyl-3-propyl-6,7-dihydro-1H-pyrazolo[4,3-d]pyrimidin-7-one (0.7 g, 3.4 mmol) in THF (25 mL) was added drop wise to the above reaction mixture at 0° C. The reaction mixture was stirred for 10 min at room temperature. The reaction mixture was cooled and a solution of ethyl(2S)-2-ethoxy-3-[4-2-hydroxyethoxy)phenyl]propanoate (1.054 g, 3.79 mmol), obtained in preparation 9, in THF (10 mL) was added to the reaction mixture slowly. The reaction mixture was allowed to stir at room temperature for 3 days. THF was removed under reduced pressure and the residue was purified by column chromatography using 15% ethyl acetate in pet ether to get the title compound (0.8 g, 50%).

¹H NMR (CDCl₃): δ 1.00 (t, J=7.3 Hz, 3H), 1.17 (t, J=6.8 Hz, 3H), 1.23 (t, J=6.8 Hz, 3H), 1.72-1.84 (m, 2H), 2.70 (s, 3H), 2.92-3.02 (m, 4H), 3.25-3.42 (m, 1H), 3.52-3.70 (m, 1H), 3.97 (t, J=6.6 Hz, 1H), 4.15 (s, 3H), 4.17 (q, J=6.6 Hz, 2H), 4.39 (t, J=4.7 Hz, 2H), 4.91 (t, 3=4.4 Hz, 2H), 6.87 (d, J=8.3 Hz, 2H), 7.18 (d, J=8.3 Hz, 2H).

### EXAMPLE 6

### 2(S)-3-[4-{2(1,5-Dimethyl-3-propyl-1H-pyrazolo[4,3-d]pyrimidin-7-yloxy)ethoxy}phenyl]-2-ethoxypropanoic Acid

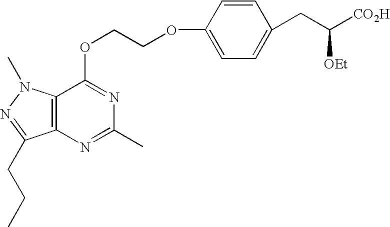

The title compound (0.3 g, 40%) was obtained as white solid from ethyl 2(S)-3-[4-{2-(1,5-dimethyl-3-propyl-1H-pyrazolo[4,3-d]pyrimidin-7-yloxy)ethoxy}-phenyl]-2-ethoxypropanoate (0.8 g, 1.7 mmol) obtained in example 5 by hydrolyzing in methanol-water (2:1, 30 mL) using sodium carbonate (0.9 g, 8.5 mmol) at room temperature for 48 h following a similar procedure as described in example 2, mp 148-150° C.

[α]_{D}²⁵=−7.6° (c=0.5, CHCl₃).

¹H NMR (CDCl₃): δ 1.00 (t, J=7.3 Hz, 3H), 1.20 (t, J=7.0 Hz, 3H), 1.75-1.93 (m, 2H), 2.69 (s, 3H), 2.17-3.10 (m, 4H), 3.45-3.65 (m, 2H), 4.06 (dd, J=7.0 and 4.7 Hz, 1H), 4.14 (s, 3H), 4.39 (t, J=4.6 Hz, 2H), 4.91 (t, J=5.1 Hz, 2H), 6.88 (d, J=8.6 Hz, 2H), 7.18 (d, J=8.6 Hz, 2H).

### EXAMPLE 7

### Ethyl 2-ethoxy-3-[4-{2-(1-methyl-5-phenyl-3-propyl-1H-pyrazolo-[4,3-d]-pyrimidin-7-yloxy)ethoxy}phenyl]propanoate

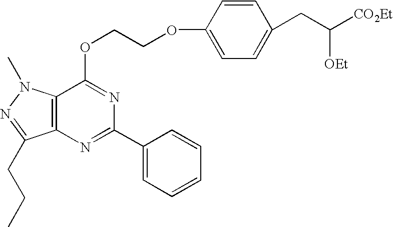

The title compound (480 mg, 96.7%) was obtained as white solid by condensing 1-methyl-5-phenyl-3-propyl-6,7-dihydro-1H-pyrazolo[4,3-d]pyrimidin-7-one, obtained in preparation 11, (250 mg, 0.93 mmol) with ethyl 3-[4-(2-bromoethoxy)phenyl]-2-ethoxypropanoate obtained in step ii. Preparation 6 (386 mg, 1.12 mmol) was taken in dry N,N-dimethylformamide (10 mL) in the presence of anhydrous K₂CO₃ (386 mg, 2.79 mmol) at room temperature for 12 h following the similar procedure as described in example 1, mp 80-82° C.

¹H NMR (CDCl₃): δ 1.04 (t, J=7.3 Hz, 3H1), 1.17 (t, J=7.1 Hz, 3H), 1.23 (t, J=6.8 Hz, 3H), 1.84-2.01 (m, 2H), 2.92-3.09 (m, 4H), 3.28-3.44 (m, 1H), 3.52-3.70 (m, 1H), 3.97 (t, J=6.6 Hz, 1), 4.17 (q, J=7.1 Hz, 2H), 4.19 (s, 3H), 4.45 (t, J=4.7 Hz, 2H), 5.05 (t, J=4.6 Hz, 2H), 6.88 (d, J=8.8 Hz, 2H), 7.18 (d, J=8.8 Hz, 2H), 7.40-7.55 (m, 3H), 8.42-8.55 (m, 2H).

### EXAMPLE 8

### 3-[4-{2-(1-Methyl-5-phenyl-3-propyl-1H-pyrazolo-14,3-d]pyrimidin-7-yloxy)ethoxy}phenyl-2-ethoxypropanoic Acid

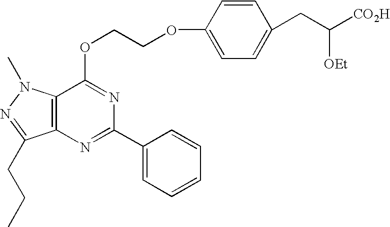

The title compound (149 mg, 63%) obtained as a white solid from ethyl 2-ethoxy-3-[4-{2-(1-methyl-5-phenyl-3-propyl-1H-pyrazolo-[4,3-d]pyrimidin-7-yloxy)-ethoxy}phenyl]propanoate (253 mg, 0.47 mmol) obtained in example 7 by hydrolyzing in methanol-water (3:2, 8 mL) using sodium carbonate (252 mg, 2.37 mmol) at room temperature for 24 h following the similar procedure as described in example 2, mp 124-126° C.

¹H NMR (CDCl₃): δ 1.04 (t, J=7.3 Hz, 3H), 1.19 (t, J=7.0 Hz, 3H), 1.84-2.01 (m, 2H), 2.92-3.17 (m, 4H), 3.42-3.69 (m, 2H), 4.06 (t, J=5.6 Hz, 1H), 4.19 (s, 3H), 4.47 (t, J=4.7 Hz, 2H), 5.06 (t, J=4.8 Hz, 2H), 6.89 (d, J=8.8 Hz, 2H), 7.18 (d, J=8.3 Hz, 2H), 7.39-7.56 (m, 3H), 8.48 (d, J=8.3 Hz, 2H).

### EXAMPLE 9

### Ethyl(2S)-3-[4-{2-(5-cyclopropyl-1-methyl-3-propyl-1H-pyrazolo[4,3-d]pyrimidin-7-yloxy)ethoxy}phenyl]-2-ethoxypropanoate

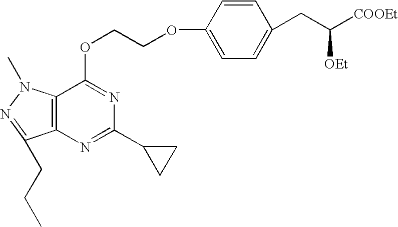

The title compound (230 g, 38%) was obtained by condensing 1-methyl-3-propyl-5-cyclopropyl-1H-pyrazolo[4,3-d]pyrimidin-7-one (200 mg, 0.86 mmol) obtained in preparation 12 with ethyl(2S)-3-[4-2-bromoethoxy)phenyl]-2-ethoxypropanoate (357 mg, 1.03 mmol) obtained in preparation 5 in dry N,N-dimethylformamide (8 mL) in the presence of anhydrous K₂CO₃ (357 mg, 2.58 mmol) at room temperature for 24 h following the similar procedure as described in example 3.

¹H NMR (CDCl₃): δ 1.01 (t, J=7.3 Hz, 3H), 1.05-1.31 (m, 10H), 1.71-1.93 (m, 2H), 2.17-2.31 (m, 1H), 2.87-3.05 (m, 4H), 3.28-3.43 (m, 1H), 3.51-3.68 (m, 1H), 3.97 (t, J=7.3 Hz, 1H), 4.12 (s, 3H), 4.17 (q, J=7.1 Hz, 21), 4.36 (t, J=4.4 Hz, 2H), 4.83 (t, J=4.4 Hz, 2H), 6.86 (d, J=8.3 Hz, 2H), 7.17 (d, J=8.3 Hz, 2H).

### EXAMPLE 10

### (2S)-3-[4-{2-(5-Cyclopropyl-1-methyl-3-propyl-1H-pyrazolo[4,3-d]pyrimidin-7-yloxy)ethoxy}phenyl]-2-ethoxypropanoic Acid

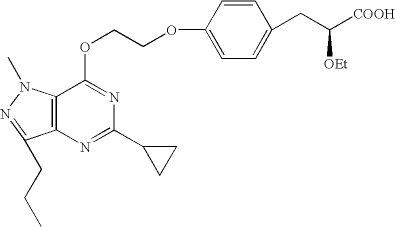

The title compound (145 mg, 81%) was obtained as a white solid from ethyl(2S)-3-[4-{2-(5-cyclopropyl-1-methyl-3-propyl-1H-pyrazolo[4,3-d]pyrimidin-7-yloxy)-ethoxy]phenyl]-2-ethoxypropanoate (190 mg, 0.38 mmol) obtained in example 9 by hydrolyzing in methanol-water (5:2, 7 mL) using sodium carbonate (203 mg, 1.9 mmol) at room temperature for 18 h following the similar procedure as described in example 2, mp 118-120° C.

[α]_{D}²⁵=−14.0° (c=0.5, CH₃OH).

¹H NMR (CDCl₃): δ 0.93-1.13 (m, 7H), 1.18 (t, J=7.1 Hz, 3H), 1.72-1.93 (m, 2H), 2.17-2.31 (m, 1H), 2.93 (t, J=7.6 Hz, 2H), 2.96-3.14 (m, 2H), 3.40-3.68 (m, 2H), 4.02-4.08 (m, 1H), 4.10 (s, 3H), 4.35 (t, J=4.6 Hz, 2H), 4.83 (t, J=4.6 Hz, 2H), 6.85 (d, J=8.3 Hz, 2H), 7.17 (d, J=8.3 Hz, 2H).

### EXAMPLE 11

### Ethyl 6-[5-ethyl-1-methyl-3-propyl-1H-pyrazolo[4,3-d]pyrimidin-7-yloxy-methyl{4-(1-ethyloxycarbonylbutoxy)phenyl}carboxamido]hexanoate

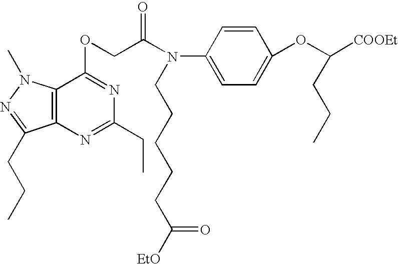

The title compound (400 mg, 55%) was obtained by condensing 5-ethyl-1-methyl-3-propyl-6,7-dihydro-1H-pyrazolo[4,3-d]-pyrimidin-7-one obtained in preparation 23 (250 mg, 1.14 mmol) with ethyl 6-chloromethyl[4-(1-ethyloxycarbonylbutoxy)phenyl]carboxamidohexanoate, obtained in preparation 16, in dry N,N-dimethylformamide (10 mL) in the presence of anhydrous K₂CO₃ (470 mg, 3.41 mmol) at room temperature for 96 h following a similar procedure as described in example 1.

¹H NMR (CDCl₃): δ 1.00 (t, J=7.3 Hz, 6H), 1.23 (t, J=7.1 Hz, 3H), 1.24-1.38 (m, 10H), 1.43-1.70 (m, 4H), 1.75-2.00 (m, 4H), 2.26 (t, J=7.6 Hz, 2H), 2.82-2.98 (m, 4H), 3.67 (t, J=7.3 Hz, 2H), 4.09 (q, J=7.2 Hz, 2H), 4.17 (q, J=7.3 Hz, 2H), 4.19 (s, 3H), 4.63 (t, J=5.8 Hz, 1H), 4.81 (s, 2H), 6.95 (d, J=8.8 Hz, 2H), 7.22 (d, J=8.8 Hz, 2H).

### EXAMPLE 12

### 6-[4-(1-Carboxybutoxy)phenyl(5-ethyl-1-methyl-3-propyl-1H-pyrazolo [4,3-d]pyrimidin-7-yloxymethyl)carboxamido]hexanoic Acid

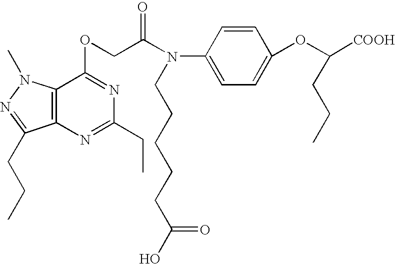

The title compound (200 mg, 73%) was obtained from ethyl 6-[5-ethyl-1-methyl-3-propyl-1H-pyrazolo[4,3-d]pyrimidin-7-yloxymethyl{4-(1-ethyloxycarbonyl-butoxy)phenyl}carboxamido]hexanoate (300 mg, 0.47 mmol), obtained in example 11, by hydrolyzing in methanol-water using sodium carbonate (248 mg, 2.35 mmol) at room temperature for 18 h following a similar procedure as described in example 2.

¹H NMR (CDCl₃): δ 0.98 (t, J=7.3 Hz, 31), 1.02 (t, J=7.3 Hz, 3H), 1.22-1.42 (m, 7H), 1.43-1.88 (m, 6H), 1.92-2.11 (m, 2H), 2.31 (t, J=6.8 Hz, 2H), 2.80-2.99 (m, 4H), 3.58-3.77 (m, 21), 4.17 (s, 31), 4.63 (t, J=5.9 Hz, 1H), 4.77 (d, I=5.2 Hz, 1H), 4.91 (d, J=4.8 Hz, 1H), 6.98 (d, J=8.8 Hz, 2H), 7.21 (d, J=8.8 Hz, 2H).

### EXAMPLE 13

### Bis-arginine Salt of 6-[4-(1-carboxybutoxy)phenyl(5-ethyl-1-methyl-3-propyl-1H-pyrazolo[4,3-d]pyrimidin-7-yloxymethyl)carboxamido]hexanoic Acid

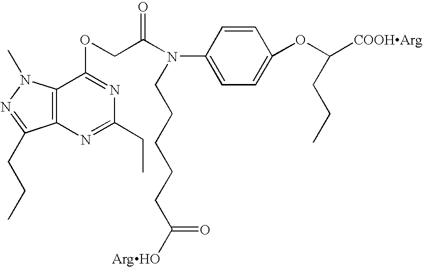

To a solution of 6-[4-(1-carboxybutoxy)phenyl(5-ethyl-1-methyl-3-propyl-1H-pyrazolo[4,3-d]pyrimidin-7-yloxymethyl)carboxamido]hexanoic acid obtained in example 12 (72 mg, 0.123 mmol) in ethanol (2 mL) was added arginine (43 mg, 0.246 mmol) dissolved in ethanol (1 mL) and stirred the reaction mixture at room temperature for 18 h. Ethanol was then distilled off under vacuum, flushed with toluene (3×3 mL), dried under high vacuum to yield the title compound as a hygroscopic solid (110 mg, 96%), mp 158-160° C.

¹H NMR (CD₃OD): δ 0.80-1,03 (m, 6H), 1.22-1.90 (m, 17H), 2.15 (t, J=6.8 Hz, 2H), 2.83-2.99 (m, 2H), 3.10-3.37 (m, 8H), 3.41-3.75 (m, 6H), 4.16 (s, 3H), 4.63-4.92 (m, 5H), 7.02 (d, J=8.8 Hz, 2H), 7.21 (d, I=9.2 Hz, 2H).

### EXAMPLE 14

### 3-[4-{2-(1,5-Dimethyl-3-propyl-1H-pyrazolo [4,3-d]pyrimidin-7-yloxy)ethoxy}phenyl]-2-ethoxypropanoic Acid

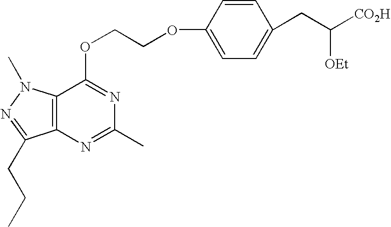

To a cooled solution of triphenyl phosphine (420 mg, 1.6 mmol) in dry THF (4 mL) DIAD (0.32 mL, 1.6 mmol) was added drop wise and stirred for 15 min. 1,5-Dimethyl-3-propyl-6,7-dihydro-1H-pyrazolo[4,3-d]pyrimidin-7-one (165 mg, 0.8 mmol) in THF (6 mL) was added drop wise to the above reaction mixture at 0° C. The reaction mixture was stirred for 10 min at room temperature and cooled. To this ethyl 2-ethoxy-3-[4-(2-hydroxyethoxy)phenyl]propanoate (248 mg, 0.881 mmol), obtained in preparation 7, in THF (5 mL) was added slowly. The reaction mixture was allowed to stir at room temperature for 4 days. THF was removed under reduced pressure and the residue was purified by column chromatography using 10% ethyl acetate in pet ether to get the corresponding ester of the title compound.

To a solution of compound obtained above in methanol (4 mL) lithium hydroxide (44 mg, 1.05 mmol) in water (1 mL) was added and stirred at room temperature for 18 h. Methanol was removed from the reaction mixture under reduced pressure and the resulting aqueous layer was acidified with 2N HCl at 0° C. to pH-2. The aqueous layer was extracted with ethyl acetate, dried (Na₂SO₄) and concentrated. Column chromatography of the residue, using 60% ethyl acetate in pet ether as eluent, afforded the title compound (50 mg), mp 142-144° C.

¹H NMR (CDCl₃): δ 0.98 (t, J=7.3 Hz, 3H), 1.18 (t, J=7.1 Hz, 3H), 1.73-1.92 (m, 2H), 2.68 (s, 3H), 2.90 (t, J=7.6 Hz, 2H), 2.98-3.15 (m, 2H), 3.43-3.68 (m, 21), 4.06 (dd, J=7.1 and 4.7 Hz, 1H), 4.13 (s, 3H), 4.37 (t, J=4.4 Hz, 2H), 4.90 (t, J=4.0 Hz, 2H), 6.86 (d, J=8.8 Hz, 2H), 7.17 (d, J=8.3 Hz, 2H).

### EXAMPLE 15

### 3-[4-{2-(5-Ethyl-1-methyl-3-propyl-1H-pyrazolo[4,3-d]pyrimidin-7-yloxy)ethoxy}phenyl]-2-ethoxypropanoic Acid

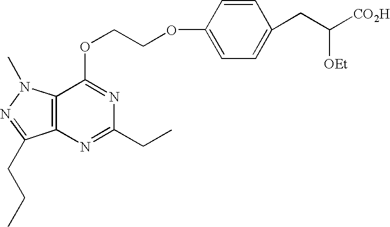

The title compound (65 mg, 9%) was prepared from 5-ethyl-1-methyl-3-propyl-6,7-dihydro-1H-pyrazolo[4,3-d]pyrimidin-7-one (0.35 g, 1.6 mmol) following the similar procedure as described in the example 14, mp 140-142° C.

¹H NMR (CDCl₃): δ 1.00 (t, J=7.3 Hz, 3H), 1.20 (t, J=7.1 Hz, 3H), 1.37 (t, J=7.6 Hz, 3H), 1.75-1.91 (m, 2H), 2.86-3.10 (m, 6H), 3.42-3.68 (m, 2H), 4.06 (dd, J=7.3 and 4.4 Hz, 1H), 4.14 (s, 31), 4.40 (t, J=4.4 Hz, 2H), 4.93 (t, J=4.9 Hz, 2H), 6.88 (d, J=8.3 Hz, 2H), 7.18 (d, J=8.3 Hz, 2H).

### EXAMPLE 16

### Ethyl 3-[4-{1,5-dimethyl-3-propyl-1H-pyrazolo[4,3-d]pyrimidin-7-yloxy-methyl(4-fluorobenzyl)carboxamido}phenyl]-2-ethoxypropanoate

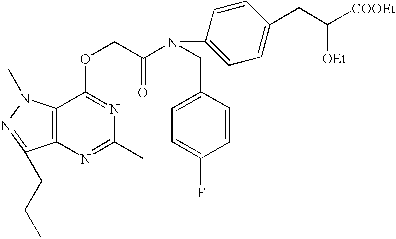

The title compound (170 mg, 47%) was obtained as white solid by condensing 1,5-dimethyl-3-propyl-6,7-dihydro-1H-pyrazolo[4,3-d]pyrimidin-7-one obtained in preparation 24 (125 mg, 0.606 mmol) with ethyl 3-[4-chloromethyl(4-fluorobenzyl)carboxamidophenyl]-2-ethoxypropanoate obtained in preparation 20 in dry N,N-dimethylformamide (7 mL) in the presence of anhydrous K₂CO₃ (251 mg, 1.82 mmol) at room temperature for 24 h following a similar procedure as described in example 1, mp 86-88° C.

¹H NMR (CDCl₃): δ 1.00 (t, J=7.3 Hz, 3H), 1.15 (t, J=6.8 Hz, 3H), 1.23 (t, J=7.1 Hz, 3H), 1.87-1.94 (m, 2H), 2.61 (s, 3H), 2.94 (t, J=7.5 Hz, 2H), 3.00-3.09 (m, 2H), 3.30-3.44 (m, 1H), 3.55-3.73 (m, 1H), 3.97-4.27 (m, 6H), 4.82 (s, 2H), 4.85 (s, 2H), 6.95 (t, J=8.5 Hz, 2H), 7.08 (d, J=8.3 Hz, 2H), 7.12-7.34 (m, 4H).

### EXAMPLE 17

### 3-[4-{1,5-Dimethyl-3-propyl-1H-pyrazolo[4,3-d]pyrimidin-7-yloxymethyl(4-fluorobenzyl)carboxamido}phenyl]-2-ethoxypropanoic Acid

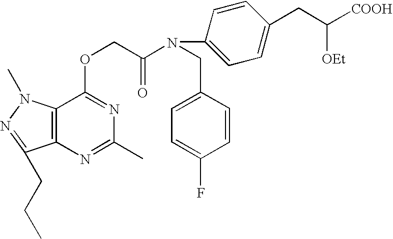

The title compound (59 mg, 86%) obtained as white solid from Ethyl 3-[4-{1,5-dimethyl-3-propyl-1H-pyrazolo[4,3-d]pyrimidin-7-yloxy-methyl(4-fluorobenzyl)carboxamido}phenyl]-2-ethoxypropanoate (72 mg, 122 μmol) obtained in example 16 by hydrolyzing in methanol-water (3:1, 4 mL) using sodium carbonate (64 mg, 0.61 mmol) at room temperature for 18 h by following a similar procedure as described in example 2.

¹H NMR (CDCl₃): δ 0.99 (t, J=7.3 Hz, 3H), 1.15 (t, J=7.3 Hz, 3H), 1.72-1.88 (m, 2H), 2.60 (s, 3H), 2.94 (t, J=7.1 Hz, 2H), 3.01-3.17 (m, 2H), 3.35-3.73 (m, 2H), 4.03-4.21 (m, 4H), 4.78 (s, 2H), 4.86 (s, 2H), 6.87 (d, J=8.3 Hz, 1H), 6.96 (t, J=8.8 Hz, 2H), 7.05-7.38 (m, 5H).

### EXAMPLE 18

### Ethyl 2-(4-[5-ethyl-1-methyl-3-propyl-1H-pyrazolo[4,3-]pyrimidin-7-yloxy-methyl(4-(4-ethyloxycarbonylphenyl)butyl}carboxamido]phenoxy)pentanoate

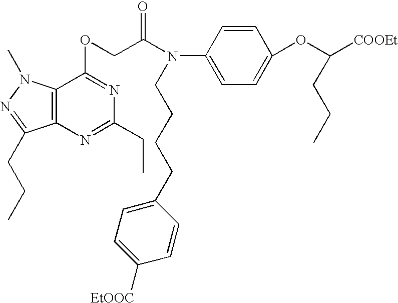

The title compound (530 mg, 86%) was obtained by condensing 5-ethyl-1-methyl-3-propyl-6,7-dihydro-1H-pyrazolo[4,3-d]-pyrimidin-7-one obtained in preparation 23 (193 mg, 0.88 mmol) with ethyl 2-[4-chloromethyl {4-4-ethyloxycarbonylphenyl)butyl}carboxamidophenoxy]pentanoate (530 mg, 1.03 mmol), obtained in preparation 22, in dry N,N-dimethylformamide (8 mL) in the presence of anhydrous K₂CO₃ (358 mg, 2.59 mmol) at room temperature for 48 h following a similar procedure as described in example 1.

¹H NMR (CDCl₃): δ 1.00 (t, J=7.3 Hz, 6H), 1.27 (t, J=7.1 Hz, 3H), 1.32-1.44 (m, 6H), 1.47-1.72 (m, 6H), 1.77-2.02 (m, 4H), 2.65 (t, J=6.8 Hz, 2H), 2.81-2.94 (m, 4H), 3.70 (t, J=6.4 Hz, 2H), 4.19 (s, 3H), 4.25 (q, J=7.3 Hz, 2H), 4.36 (q, J=7.3 Hz, 2H), 4.62 (t, J=6.1 Hz, 1H), 4.80 (s, 2H), 6.93 (d, 1=8.8 Hz, 2H), 7.17 (d, J=7.8 Hz, 2H), 7.19 (d, J=8.8 Hz, 211), 7.92 (d, J=7.8 Hz, 2H).

### EXAMPLE 19

### 2-[4-{4-(4-Carboxyphenyl)butyl(5-ethyl-1-methyl-3-propyl-1H-pyrazolo [4,3-d]pyrimidin-7-yloxymethyl)carboxamido}phenoxy]pentanoic Acid

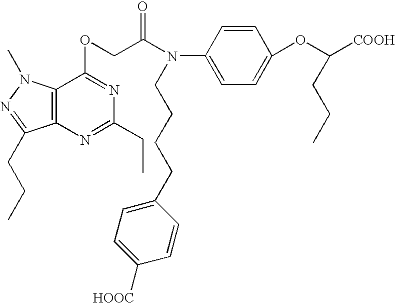

The title compound (81 mg, 88%) obtained as white solid from ethyl 2-(4-[5-ethyl-1-methyl-3-propyl-1H-pyrazolo[4,3-d]pyrimidin-7-yloxymethyl {4-(4-ethyloxycarbonylphenyl)butyl}carboxamido]phenoxy)pentanoate (100 mg, 142 μmol), obtained in example 18, by hydrolyzing in methanol-water using sodium carbonate (76 mg, 0.71 mmol) at room temperature for 18 h by following a similar procedure as described in example 2, mp 0.178-180° C.

¹H NMR (CD₃OD+DMSO-d₆): δ 1.06-1.11 (m, 6H), 1.45 (t, J=7.5 Hz, 3H), 1.62-2.18 (m, 10H), 2.74-3.12 (m, 61), 3.89 (t, J=5.4 Hz, 2H), 4.30 (s, 31), 4.90 (br s, 1H), 5.06 (s, 2H), 7.16 (d, J=8.6 Hz, 2H), 7.36 (d, J=8.1 Hz, 2H), 7.46 (d, J=8.6 Hz, 2H), 8.00 (d, J=8.1 Hz, 2H).

The compounds of the present invention lowered random blood sugar level, triglyceride, total cholesterol, LDL, VLDL and increased HDL. This was demonstrated by in vitro as well as in vivo animal experiments.

Demonstration of Efficacy of Compounds

A) In Vitro:

a) Determination of hPPARα Activity

Ligand binding domain of hPPARα was fused to DNA binding domain of Yeast transcription factor GAL4 in eucaryotic expression vector. Using superfect (Qiagen, Germany) as transfecting reagent HEK-293 cells were transfected with this plasmid and a reporter plasmid harboring the luciferase gene driven by a GAL4 specific promoter. Compound was added at different concentrations after 42 hrs of transfection and incubated overnight. Luciferase activity as a function of compound binding/activation capacity of PPARα was measured using Packard Luclite kit (Packard, USA) in Top Count (Ivan Sadowski, Brendan Bell, Peter Broag and Melvyn Hollis. Gene 1992, 118: 137-141; Superfect Transfection Reagent Handbook. February 1997. Qiagen, Germany).

b) Determination of hPPARγ Activity

Ligand binding domain of hPPARγ1 was fused to DNA binding domain of Yeast transcription factor GAL4 in eucaryotic expression vector. Using lipofectamine (Gibco BRL, USA) as transfecting reagent HEK-293 cells were transfected with this plasmid and a reporter plasmid harboring the luciferase gene driven by a GAL4 specific promoter. Compound was added at 1 μM concentration after 48 hrs of transfection and incubated overnight. Luciferase activity as a function of drug binding/activation capacity of PPARγ1 was measured using Packard Luclite kit (Packard, USA) in Packard Top Count (Ivan Sadowski, Brendan Bell, Peter Broag and Melvyn Hollis. Gene 1992, 118: 137-141; Guide to Eukaryotic Transfections with Cationic Lipid Reagents. Life Technologies, GIBCO BRL, USA).

| Example No. | Concentration | PPARα | PPARγ | Concentration |
|---|---|---|---|---|
| 2 | 50 μM | 2.7 | 1 μM | 1.4 |
| 4b | 50 μM | 5.1 | 1 μM | 6.9 |

c) Determination of HMG CoA Reductase Inhibition Activity

Liver microsome bound reductase was prepared from 2% cholestyramine fed rats at mid-dark cycle. Spectrophotometric assays were carried out in 100 mM KH₂PO₄, 4 mM DTT, 0.2 mM NADPH, 0.3 mM HMG CoA and 125 μg of liver microsomal enzyme. Total reaction mixture volume was kept as 1 ml. Reaction was started by addition of HMG CoA. Reaction mixture was incubated at 37° C. for 30 min and decrease in absorbance at 340 nm was recorded. Reaction mixture without substrate was used as blank (Goldstein, J. L and Brown, M. S. Progress in understanding the LDL receptor and HMG CoA reductase, two membrane proteins that regulate the plasma cholesterol J. Lipid Res 1984, 25: 1450-1461). The test compounds inhibited the HMG CoA reductase enzyme.

B) In Vivo

a) Efficacy in Genetic Models

Mutation in colonies of laboratory animals and different sensitivities to dietary regimens have made the development of animal models with non-insulin dependent diabetes and hyperlipidemia associated with obesity and insulin resistance possible. Genetic models such as db/db and ob/ob (Diabetes, (1982) 31(1): 1-6) mice and zucker fa/fa rats have been developed by the various laboratories for understanding the pathophysiology of disease and testing the efficacy of new antidiabetic compounds (Diabetes, (1983) 32: 830-838; Annu. Rep. Sankyo Res. Lab. (1994). 46: 1-57). The homozygous animals, C57 BL/KsJ-db/db mice developed by Jackson Laboratory, US, are obese, hyperglycemic, hyperinsulinemic and insulin resistant (J. Clin. Invest., (1990) 85: 962-967), whereas heterozygous are lean and normoglycemic. In db/db model, mouse progressively develops insulinopenia with age, a feature commonly observed in late stages of human type II diabetes when blood sugar levels are insufficiently controlled. The state of pancreas and its course vary according to the models. Since this model resembles that of type II diabetes mellitus, the compounds of the present invention were tested for blood sugar and triglycerides lowering activities.

Male C57BL/KsJ-db/db mice of 8 to 14 weeks age, having body weight range of 35 to 60 grams, bred at Dr. Reddy's Research Foundation (DRF) animal house, were used in the experiment The mice were provided with standard feed (National Institute of Nutrition (NIN), Hyderabad, India) and acidified water, ad libitum. The animals having more tan 350 mg/dl blood sugar were used for testing. The number of animals in each group was 4.

Test compounds were suspended on 0.25% carboxymethyl cellulose and administered to test group at a dose of 0.1 mg to 30 mg/kg through oral gavage daily for 6 days. The control group received vehicle (dose 10 ml/kg). On 6th day the blood samples were collected one hour after administration of test compounds/vehicle for assessing the biological activity.

The random blood sugar and triglyceride levels were measured by collecting blood (100 μl) through orbital sinus, using heparinised capillary in tubes containing EDTA which was centrifuged to obtain plasma. The plasma glucose and triglyceride levels were measured spectrometrically, by glucose oxidase and glycerol-3-PO₄ oxidase/peroxidase enzyme (Dr. Reddy's Lab. Diagnostic Division Kits, Hyderabad, India) methods respectively.

The blood sugar and triglycerides lowering activities of the test compound was calculated according to the formula.

No adverse effects were observed for any of the mentioned compounds of invention in the above test.

| | Dose (mg/ | Reduction in Blood | Triglyceride |
|---|---|---|---|
| Compound | kg) | Glucose Level (%) | Lowering (%) |
| Example 4a | 10 | 48 | 69 |
| Example 4b | 10 | 56 | 17 |
| Example 5 | 3 | 61 | 51 |

The ob/ob mice were obtained at 5 weeks of age from Bomholtgard, Denmark and were used at 8 weeks of age. Zucker fa/fa fatty rats were obtained from IffaCredo, France at 10 weeks of age and were used at 13 weeks of age. The animals were maintained under 12 hour light and dark cycle at 25±1° C. Animals were given standard laboratory chow (NIN, Hyderabad, India) and water, ad libitum (Fujiwara, T., Yoshioka, S., Yoshioka, T., Ushiyama, I and Honikoshi, H. Characterization of new oral antidiabetic agent CSO-045. Studies in KK and ob/ob mice and Zucker fatty rats. Diabetes 1988, 37: 1549-1558).

The test compounds were administered at 0.1 to 30 mg/kg/day dose for 9 days. The control animals received the vehicle (0.25% carboxymethylcellulose, dose 10 ml/kg) through oral gavage.

The blood samples were collected in fed state 1 hour after drug administration on 0 and 9 day of treatment. The blood was collected from the retro-orbital sinus through heparinised capillary in EDTA containing tubes. After centrifugation, plasma sample was separated for triglyceride, glucose, free fatty acid, total cholesterol and insulin estimations. Measurement of plasma triglyceride, glucose, total cholesterol were done using commercial kits (Dr. Reddy's Laboratory, Diagnostic Division, India). The plasma free fatty acid was measured using a commercial kit from Boehringer Mannheim, Germany. The plasma insulin was measured using a RIA kit (BARC, India). The reduction of various parameters examined are calculated according to the formula given below.

In ob/ob mice oral glucose tolerance test was performed after 9 days treatment. Mice were fasted for 5 hrs and challenged with 3 gm/kg of glucose orally. The blood samples were collected at 0, 15, 30, 60 and 120 min for estimation of plasma glucose levels.

The experimental results from the db/db mice, ob/ob mice, Zucker fa/fa rats suggest that the novel compounds of the present invention also possess therapeutic utility as a prophylactic or regular treatment for diabetes, obesity, cardiovascular disorders such as hypertension, hyperlipidaemia and other diseases; as it is known from the literature that such diseases are interrelated to each other.

Blood glucose level and triglycerides are also lowered at doses greater than 10 mg/kg. Normally, the quantum of reduction is dose dependent and plateaus at certain dose.

b) Plasma Triglyceride and Cholesterol Lowering Activity in Hypercholesterolemic Rat Models

Male Sprague Dawley rats (NIN stock) were bred in DRF animal house. Animals were maintained under 12 hour light and dark cycle at 25±1° C. Rats of 180-200 gram body weight range were used for the experiment Animals were made hypercholesterolemic by feeding 2% cholesterol and 1% sodium cholate mixed with standard laboratory chow [National Institute of Nutrition (NIN), Hyderabad, India] for 6 days. Throughout the experimental period the animals were maintained on the same diet (Petit, D., Bonnefis, M. T., Rey, C and Infante, R. Effects of ciprofibrate on liver lipids and lipoprotein synthesis in normal and hyperlipidemic rats. Atherosclerosis 1988, 74: 215-225).

The test compounds were administered orally at a dose 0.1 to 30 mg/kg/day for 3 days. Control group was treated with vehicle alone (0.25% Carboxymethylcellulose; dose 10 ml/kg).

The blood samples were collected in fed state 1 hour after drug administration on 0 and 3 day of compound treatment. The blood was collected from the retro-orbital sinus through heparinised capillary in EDTA containing tubes. After centrifugation, plasma sample was separated for total cholesterol, HDL and triglyceride estimations. Measurement of plasma triglyceride, total cholesterol and HDL were done using commercial kits (Dr. Reddy's Laboratory, Diagnostic Division, India). LDL and VLDL cholesterol were calculated from the data obtained for total cholesterol, HDL and triglyceride. The reduction of various parameters examined are calculated according to the formula.

c) Plasma Triglyceride and Total Cholesterol Lowering Activity in Swiss Albino Mice and Guinea Pigs

Male Swiss albino mice (SAM) and male Guinea pigs were obtained from NIN and housed in DRF animal house. All these animals were maintained under 12 hour light and dark cycle at 25±1° C. Animals were given standard laboratory chow (NIN, Hyderabad, India) and water, ad libitum. SAM of 20-25 g body weight range and Guinea pigs of 500-700 g body weight range were used (Oliver, P., Plancke, M. O., Marzin, D., Clavey, V., Sauzieres, J and Fruchart, J. C. Effects of fenofibrate, gemfibrozil and nicotinic acid on plasma lipoprotein levels in normal and hyperlipidemic mice. Atherosclerosis. 1988. 70: 107-114).

The test compounds were administered orally to Swiss albino mice at 0.3 to 30 mg/kg/day dose for 6 days. Control mice were treated with vehicle (0.25% Carboxymethylcellulose; dose 10 ml/kg). The test compounds were administered orally to Guinea pigs at 0.3 to 30 mg/g/day dose for 6 days. Control animals were treated with vehicle (0.25% Carboxymethylcellulose; dose 5 ml/kg).

The blood samples were collected in fed state 1 hour after drug administration on 0 and 6 day of treatment. The blood was collected from the retro-orbital sinus through heparinised capillary in EDTA containing tubes. After centrifugation, plasma sample was separated for triglyceride and total cholesterol (Wieland, O. Methods of Enzymatic analysis. Bergermeyer, H. O., Ed., 1963. 211-214; Trinder, P. Ann. Clin. Biochem 1969, 6: 24-27). Measurement of plasma triglyceride, total cholesterol and HDL were done using commercial kits (Dr. Reddy's Diagnostic Division, Hyderabad, India).

| Compound | Dose (mg/kg) | Triglyceride Lowering (%) |
|---|---|---|
| 8 | 10 | 61 |
| 7 | 3 | 43 |
| 10 | 3 | 42 |

c) Body Weight Reducing Effect in Cholesterol Fed Hamsters

Male Syrian Hamsters were procured from NIN, Hyderabad, India. Animals were housed at DRF animal house under 12 hour light and dark cycle at 25±1° C. with free access to food and water. Animals were maintained with 1% cholesterol containing standard laboratory chow (NIN) from the day of treatment.

The test compounds were administered orally at 1 to 30 mg/kg/day dose for 15 days. Control group animals were treated with vehicle (Mill Q water, dose 10 ml/kg/day). Body weights were measured on every 3^{rd} day.

Formulae for Calculation:

1. Percent reduction in Blood sugar/triglycerides/total cholesterol were calculated according to the formula:

$$Percent reduction ( \% ) = [ 1 - \frac{TT / OT}{TC / OC} ] \times 100$$
OC=Zero day control group value
OT=Zero day treated group value
TC=Test day control group value
TT=Test day treated group value
2. LDL and VLDL cholesterol levels were calculated according to the formula:

$$\begin{matrix}LDL \text{cholesterol} in mg \text{/dl} = [ \text{Total} \text{cholesterol} - \\ HDL \text{cholesterol} - \\ \frac{\text{Triglyceride}}{5} ] mg \text{/dl}\end{matrix}$$

$$\begin{matrix}VLDL \text{cholesterol} in mg \text{/dl} = [ \text{Total} \text{cholesterol} - \\ HDL \text{cholesterol} - \\ LDL \text{cholesterol} ] mg \text{/dl} .\end{matrix}$$

1. Novel alkyl carboxylic acids of compound of the general formula (I) 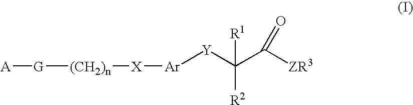
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, alkanoyl, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together form a substituted or unsubstituted 5 or 6 membered cyclic structure containing carbon atoms, a nitrogen atom and which can optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, —(CH₂)ₚO—, —(CH₂)NR⁵CO—; where R⁵ represents hydrogen or substituted or unsubstituted groups selected from alkyl, aryl, aralkyl, hydroxyalkyl, carboxyalkyl, alkanoyl, alkanoyloxy, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 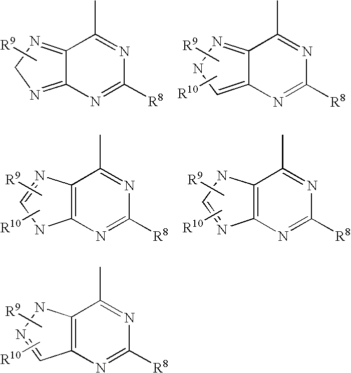
where R⁸ and R⁹, R¹⁰ when attached to carbon atom are same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives.

2. A compound according to claim 1, wherein when the groups represented by R² are substituted, the substituents are selected from halogen, hydroxy, nitro or unsubstituted or substituted groups selected from alkyl, cycloalkyl, alkoxy, cycloalkoxy, aryl, aralkyl, aralkoxyalkyl, heterocyclyl, heteroaryl, heteroaralkyl, alkanoyl, alkanoyloxy, hydroxyalkyl, amino, alkanoylamino, arylamino, aminoalkyl, aryloxy, aralkoxy, alkoxycarbonyl, alkylamino, alkoxyalkyl, aryloxyalkyl, alkylthio, thioalkyl groups, carboxylic acid or its derivatives or sulfonic acid or its derivatives.

3. A compound according to claims 1 to 2 wherein Ar represents substituted or unsubstituted groups selected from divalent phenylene, naphthylene, pyrrolyl, pyridyl, quinolinyl, benzofuryl, dihydrobenzofuryl, benzopyranyl, dihydrobenzopyranyl, indolyl, indolinyl, azaindolyl, azaindolinyl, pyrazolyl, benzothiazolyl or benzoxazolyl groups.

4. A compound according to claim 1, which is selected from:
Ethyl 2-[4-(5-ethyl-1-methyl-3-propyl-1H-pyrazolo[4,3-d]pyrimidin-7-yloxymethyl(heptyl)carboxamido)phenylsulfanyl]pentanoate or its salts in its single enantiomeric form or as a racemate;
2-[4-(5-Ethyl-1-methyl-3-propyl-1H-pyrazolo[4,3-d]pyrimidin-7-yloxymethyl-(heptyl)carboxamido)phenylsulfanyl]pentanoic acid or its salts in its single enantiomeric form or as a racemate;
Ethyl 2-ethoxy-3-[4-{2-(S-ethyl-1-methyl-3-propyl-1H-pyrazolo[4,3-d]pyrimidin-7-yloxy)ethoxy}phenyl]propanoate or its salts in its single enantiomeric form or as a racemate;
Methyl 2-ethoxy-3-[4-{2-(5-thyl-1-methyl-3-propyl-1H-pyrazolo[4,3-d]-pyrimidin-7-yloxyethoxy}phenyl]propanoate or its salts in its single enantiomeric form or as a racemate;
2-Ethoxy-3-[4-{2-(5-ethyl-1-methyl-3-propyl-1H-pyrazolo[4,3-d]pyrimidin-7-yloxy)ethoxy}phenyl]propanoic acid or its salts in its single enantiomeric form or as a racemate;
Ethyl 3-[4-{2-(1,5-dimethyl-3-propyl-1H-pyrazolo[4,3-d]pyrimidin-7-yloxy)ethoxy}phenyl]-2-ethoxypropanoate or its salts in its single enantiomeric form or as a racemate;
3-[4-{2-(1,5-Dimethyl-3-propyl-1H-pyrazolo[4,3-d]pyrimidin-7-yloxy)-ethoxy}phenyl]-2-ethoxypropanoic acid or its salts in its single enantiomeric form or as a racemate;
Ethyl 2-ethoxy-3-[4-{2-(1-methyl-5-phenyl-3-propyl-1H-pyrazolo-[4,3-d]pyrimidin-7-yloxy)ethoxy}phenyl]propanoate or its salts in its single enantiomeric form or as a racemate;
3-[4-{2-(1-Methyl-5-phenyl-3-propyl-1H-pyrazolo-[4,3-d]pyrimidin-7-yloxy)ethoxy}phenyl]-2-ethoxypropanoic acid or its salts in its single enantiomeric form or as a racemate;
Ethyl 3-[4-{2-(5-cyclopropyl-1-methyl-3-propyl-1H-pyrazolo[4,3-d]pyrimidin-7-yloxy)ethoxy}phenyl]-2-ethoxypropanoate or its salts in its single enantiomeric form or as a racemate;
3-[4-{2-(5-Cyclopropyl-1-methyl-3-propyl-1H-pyrazolo[4,3-d]pyrimidin-7-yloxy)ethoxy}phenyl]-2-ethoxypropanoic acid or its salts in its single enantiomeric form or as a racemate;
Ethyl 6-[5-ethyl-1-methyl-3-propyl-1H-pyrazolo[4,3-d]pyrimidin-7-yloxy-methyl{4-(1-ethyloxycarbonylbutoxyl)phenyl}carboxamido]hexanoate or its salts in its single enantiomeric form or as a racemate;
6-[4-(1-Carboxybutoxy)phenyl(5-ethyl-1-methyl-3-propyl-1H-pyrazolo[4,3-d]pyrimidin-7-yloxymethyl)carboxamido]hexanoic acid or its salts in its single enantiomeric form or as a racemate;
Ethyl 3-[4-{1,5-dimethyl-3-propyl-1H-pyrazolo[4,3-d]pyrimidin-7-yloxy-methyl(4-fluorobenzyl)carboxamido}phenyl]-2-ethoxypropanoate or its salts in its single enantiomeric form or as a racemate;
3-[4-{1,5-Dimethyl-3-propyl-1H-pyrazolo[4,3-d]pyrimidin-7-yloxymethyl(4-fluorobenzyl)carboxamido}phenyl]-2-ethoxypropanoic acid or its salts in its single enantiomeric form or as a racemate;
Ethyl 2-(4-[5-ethyl-1-methyl-3-propyl-1H-pyrazolo[4,3-d]pyrimidin-7-yloxy-methyl{4-(4-ethyloxycarbonylphenyl)butyl}carboxamido]phenoxy)pentanoate or its salts in its single enantiomeric form or as a racemate;
2-[4-{4-(4-Carboxyphenyl)butyl(5-ethyl-1-methyl-3-propyl-1H-pyrazolo[4,3-d]pyrimidin-7-yloxymethyl)carboxamido}phenoxy]pentanoic acid or its salts in its single enantiomeric form or as a racemate;

5. A compound according to claim 1 wherein the pharmaceutically acceptable salt is selected from the group consisting of Li, Na, K, Ca, Mg, Fe, Cu, Zn, Al, Mn; organic bases selected from N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, theobromine, glycinol, diethylamine, triethylamine, trimethylamine, tripropylamine, tromethamine, adamentyl amine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, thiamine, aminopyrimidine, aminopyridine, purine or spermidine; chiral bases like alkylphenylamine or phenyl glycinol; salts of natural amino acids selected from glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids selected from D-isomers or substituted amino acids; guanidine, substituted guanidine wherein the substituents are selected from nitro, amino, alkyl, alkenyl, alkynyl, ammonium or substituted ammonium salts. Salts include acid addition salts where appropriate which are, sulphates, nitrates, phosphates, perchlorates, borates, hydrohalides, acetates, tartrates, maleates, citrates, succinates, palmoates, methanesulfonates, benzoates, salicylates, hydroxynaphthoates, benzenesulfonates, ascorbates, glycerophosphates or ketoglutarates.

6. A process for the preparation of compound of formula (I) 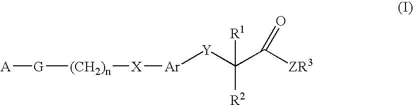
their derivatives, their analogs, their tautomeric forms, their stereoisomers, their polymorphs, their pharmaceutically acceptable salts, their pharmaceutically acceptable solvates where R¹ and R⁷ together represent a bond; R² represents hydrogen, hydroxy, halogen, substituted or unsubstituted groups selected from alkyl, cycloalkyl, cycloalkylalkyl alkoxy, aryl, alkanoyl alkanoyloxy, aroyl aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents CHR⁷; where R⁷ forms a bond together with the adjacent group R¹; m and p are integers ranging from 0-4; n is an integer in the range of 1-4; A represents pyrazolopyrimidine or imidazolopyrimidine of the formula given below: 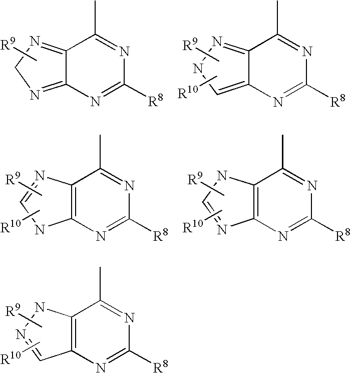
where R¹ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives, which comprises:
a) reacting the compound of formula (IIIa)
A-G-(CH₂)ₙ—X—Ar—CHO (IIIa)
where all symbols are as defined above with a compound of formula (IIIb) 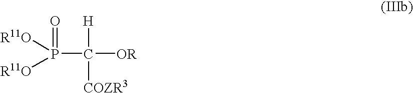
where R¹¹ represents (C₁₋₆)alkyl, R represents substituted or unsubstituted groups selected from alkyl, aryl, aralkyl, heteroaryl or heteroaralkyl and all other symbols are as defined earlier to yield compound of general formula (I) where R² represents substituted or unsubstituted groups selected from alkoxy, aryloxy, aralkoxy, heteroaryloxy, heteroaralkoxy and all other symbols are as defined above;
b) reacting the compound of formula (IIIa)
A-G-(CH₂)ₙ—X—Ar—CHO (IIIa)
where all symbols are as defined above with Wittig reagent to yield a compound of formula (I) where all symbols are as defined above; or
c) reacting the compound of formula (IIIc)
B—H (IIIc)
where B represents pyrazolopyrimidine or imidazolopyrimidine of the formula given below: 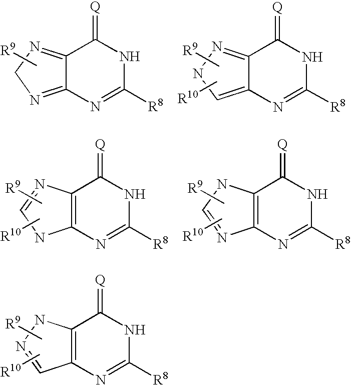
wherein Q represents O or S and all other symbols are as defined above with compound of general formula (IIId) 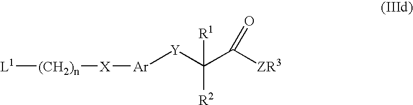
where L¹ is a leaving group; R¹ and R⁷ together represent a bond and all other symbols are as defined above to yield compound of formula (I) where all symbols are as defined above; or
d) reacting the compound of formula (IIIa)
A-G-(CH₂)ₙ—X—Ar—CHO (IIIa)
where all symbols are as defined above with a compound of formula (IIIe) 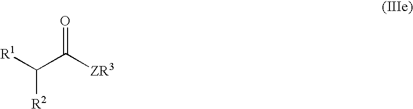
where R¹ represents hydrogen atom and all other symbols are as defined above; or
e) reacting the compound of formula (IIIg)
A-G-(CH₂)ₙ-L¹ (IIIg)
where L¹ represents a leaving group and all other symbols are as defined above with compound of formula (IIIf) 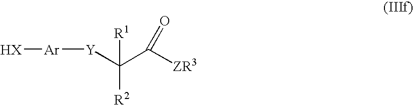
where R¹ and R⁷ together represent a bond and all other symbols are as defined above; or
f) reaction of compound of general formula (IIIh)
A-Hal (IIIh)
A is as defined above and Hal represents halogen atom with compound of formula (IIIi) 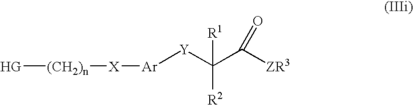
where all symbols are as defined above; or
g) reacting the of compound of general formula (IIIj)
A-G-(CH₂)ₙ—OH (IIIj)
where all symbols are as defined above with a compound of general formula (IIIf) 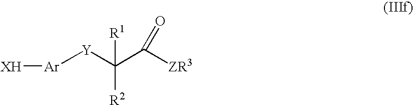
where all symbols are as defined above; or
h) reacting the compound of formula (IIIk)
A-G-(CH₂)ₙ—X—Ar—CH₂—P⁺Ph₃Hal⁻ (IIIk)
where all symbols are as defined above with a compound of formula (IIIl) 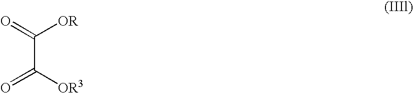
where R represents substituted or unsubstituted groups selected from alkyl, aryl, aralkyl, heteroaryl or heteroaralkyl and where R³ is as defined earlier excluding hydrogen to yield compound of general formula (I) where R² represents substituted or unsubstituted groups selected from alkoxy, aryloxy, aralkoxy, heteroaryloxy, heteroaralkoxy and all other symbols are as defined above
i) reacting the compound of formula (IIIc)
B—H (IIIc)
where B represents pyrazolopyrimidine or imidazolopyrimidine of the formula given below: 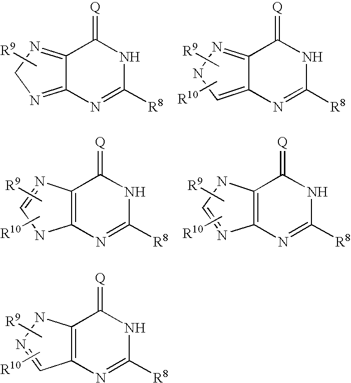
wherein Q represents O or S and all other symbols are as defined above with compound of general formula (IIIm) 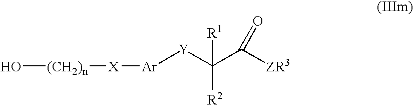
where all symbols are as defined above;
j) converting the compounds of formula (I) obtained in any of the processes described above into pharmaceutically acceptable salts or pharmaceutically acceptable solvates by conventional methods.

7. A process for the preparation of compound of formula (I) 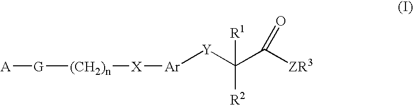
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents CHR⁷; where R⁷ represents hydrogen atom, halogen, hydroxy, alkyl, alkoxy, substituted or unsubstituted aralkyl group; m and p are integers ranging from 0-4; n is an integer in the range of 1-4; A represents pyrazolopyrimidine or imidazolopyrimidine of the formula given below: 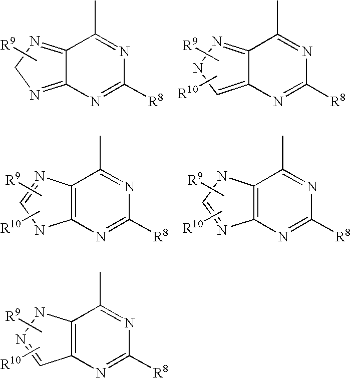
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives, which comprises:
a) reducing the compound of the formula (IVa) 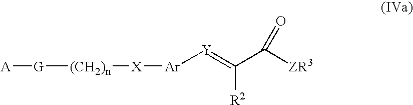
which represents a compound of formula (I) where R¹ and R⁷ together represent a bond and Z represents oxygen atom and all other symbols are as defined above to yield a compound of the general formula (I) where R¹ and R⁵ each represent hydrogen atom and all other symbols are as defined above; or
b) reacting the compound of formula (IVb) 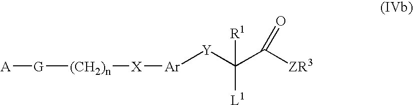
where L¹ is a leaving group, R³ is as defined earlier excluding hydrogen and all other symbols are as defined earlier with an alcohol of general formula (IVc),
R—OH (IVc)
where R represents substituted or unsubstituted groups selected from alkyl, aryl, aralkyl, heteroaryl or heteroaralkyl and all other symbols are as defined earlier to yield compound of general formula (I) where R² represents substituted or unsubstituted groups selected from alkoxy, aryloxy, aralkoxy, heteroaryloxy, heteroaralkoxy and all other symbols above; or
c) reacting the compound of formula (IIIg)
A-G-(CH₂)ₙ-L¹ (IIIg)
where all symbols are as defined above with compound of formula (IIIf) 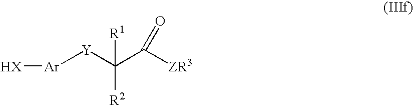
where all symbols are as defined above; or
d) reacting the compound of general formula (IIIj)
A-G-(CH₂)ₙ—OH (III)
where all symbols are as defined above with a compound of general formula (IIIf) 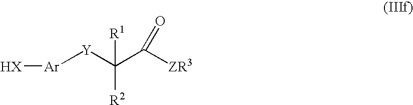
where all symbols are as defined above; or
e) reacting the compound of formula (IVd), 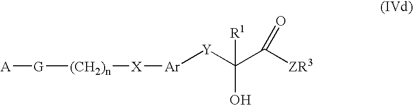
which represents a compound of formula (I), when R² represents hydroxy group and all other symbols are as defined above with a compound of formula (IVe)
R-L² (IVe)
where R represents substituted or unsubstituted groups selected from alkyl, aryl, aralkyl, heteroaryl or heteroaralkyl and L² is a halogen atom to yield compound of general formula (I) where R² represents substituted or unsubstituted groups selected from alkoxy, aryloxy, aralkoxy, heteroaryloxy, heteroaralkoxy and all other symbols are as defined above; or
f) reacting the compound of general formula (IIIh)
A-Hal (IIIh)
where Hal represents halogen atom and A is as defined earlier with the compound of formula (IIIi) 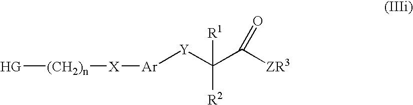
where all other symbols are as defined above; or
g) reacting the compound of general formula (IIIc)
B—H (IIIc)
where B represents pyrazolopyrimidine or imidazolopyrimidine of the formula given below: 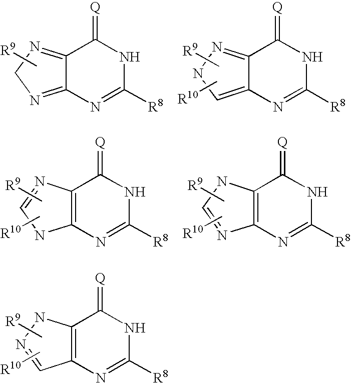
wherein Q represents O or S and all other symbols are as defined above with compound of general formula (IIIm) 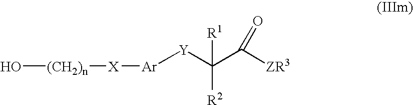
where symbols are as defined above; or
h) reacting the compound of the general formula (IIIa)
A-G-(CH₂)ₙ—X—Ar—CHO (IIIa)
where all symbols are as defined above with a compound of formula (IIIe) 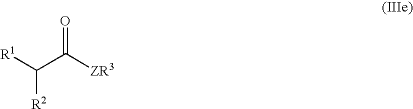
where R¹ represents hydrogen atom and all other symbols are as defined above; or
i) reacting the compound of the general formula (IIIc)
B—H (IIIc)
where B represents pyrazolopyrimidine or imidazolopyrimidine of the formula given below: 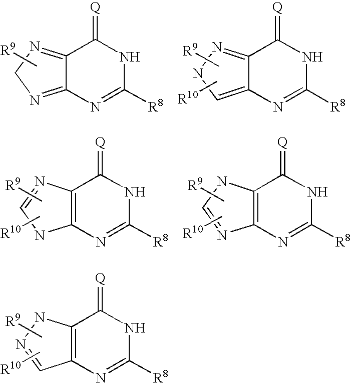
wherein Q represents O or S and all other symbols are as defined above with compound of general formula (IIId) 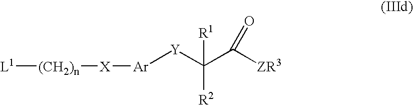
where L¹ is a leaving group and all other symbols are as defined above; or
j) converting the compound of formula (IVf) 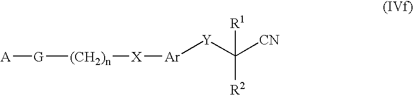
where all symbols are as defined above to a compound of formula (I) where all symbols are as defined above; or
k) reacting the compound of formula (IVg) 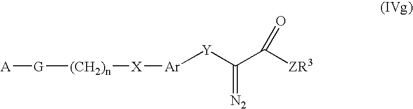
where R³ is as defined above excluding hydrogen and all other symbols are as defined above with a compound of formula (IVc)
R—OH (IVc)
where R represents substituted or unsubstituted groups selected from alkyl, aryl, aralkyl, heteroaryl or heteroaralkyl and L² is a halogen atom to yield compound of general formula (I) where R² represents substituted or unsubstituted groups selected from alkoxy, aryloxy, aralkoxy, heteroaryloxy, heteroaralkoxy and all other symbols are as defined above; or
l) converting the compounds of formula (I) obtained in any of the processes described above into pharmaceutically acceptable salts, or pharmaceutically acceptable solvates.

8. A process for the preparation of compound of formula (I) 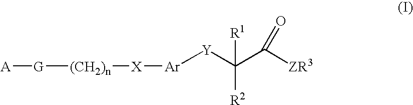
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen; Ar represents substituted or unsubstituted, divalent, single or fused, aromatic, heteroaromatic or heterocyclic group; G represents O or S; X represents NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; m and p are integers ranging from 0-4; n is an integer in the range of 1-4; A represents pyrazolopyrimidine or imidazolopyrimidine of the formula given below: 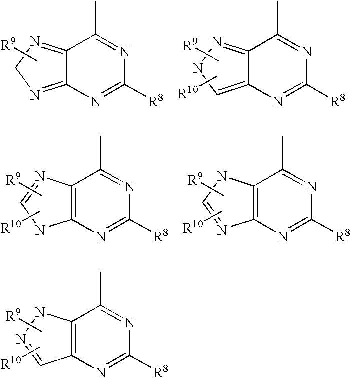
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives, which comprises:
a) reacting the compound of formula (Va) 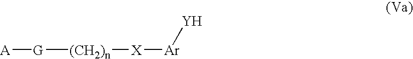
where all symbols are as defined above with compound of formula (Vb) 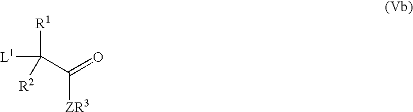
where L¹ is a leaving group and all other symbols are as defined above; or
b) reacting the compound of general formula (IIIc)
B—H (IIIc)
where B represents pyrazolopyrimidine or imidazolopyrimidine of the formula given below: 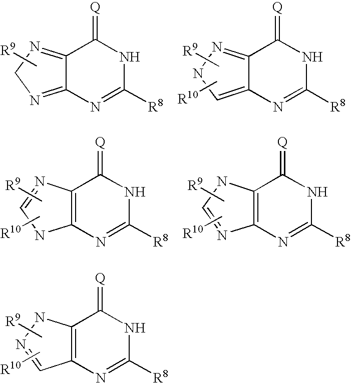
wherein Q represents O or S and all other symbols are as defined above with compound of general formula (IIId) 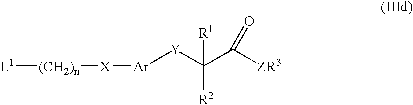
where L¹ is a leaving group and all other symbols are as defined above; or
c) reacting the compound of formula (Vc)
A-G-(CH₂)ₙ—CO-L¹ (Vc)
where L¹ represents a leaving group and all other symbols are as defined above with compound of formula (IIIf) 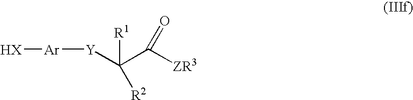
where all symbols are as defined above; or
d) reacting the compound of general formula (IIIh)
A-Hal (IIIh)
where A is as defined above and Hal represents halogen atom with the compound of formula (IIIi) 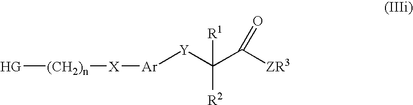
where all symbols are as defined above; or
e) reacting the compound of formula (Vd)
A-G-(CH₂)ₙ—COOH (Vd)
where all symbols are as defined above with a compound of general formula (IIIf) 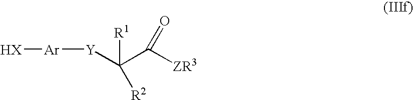
where all symbols are as defined above; or
f) reacting the compound of general formula (IIIc)
B—H (IIIc)
where B represents pyrazolopyrimidine or imidazolopyrimidine of the formula given below: 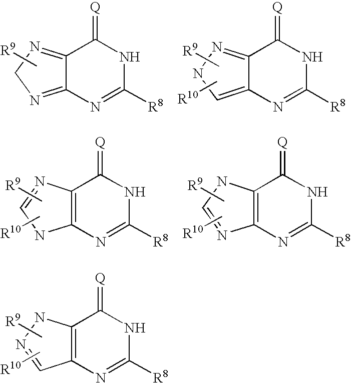
wherein Q represents O or S and all other symbols are as defined above with compound of general formula (IIIm) 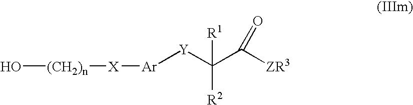
where all symbols are as defined above; or
g) converting the compound of formula (IVf) 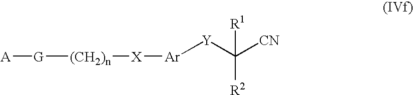
where all symbols are as defined above to a compound of formula (I) where all symbols are as defined above; or
h) reacting the compound of general formula (IIIj)
A-G-(CH₂)ₙ—OH (IIIj)
where A, G and n are as defined above with a compound of general formula (IIIf) 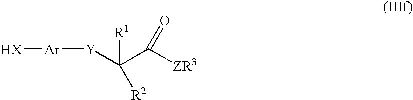
where all symbols are as defined above; or
i) converting the compounds of formula (I) obtained in any of the processes described above into pharmaceutically acceptable salts, or pharmaceutically acceptable solvates.

9. A process for the preparation of compound of formula (I) 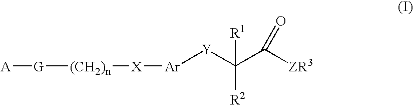
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl arylaminocarbonyl groups; R³ represents hydrogen; Z represents oxygen; Ar represents substituted or unsubstituted, divalent, single or fused, aromatic, heteroaromatic or heterocyclic group; G represents O or S; X represents O, NHR⁵, —CO(C₂)NR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 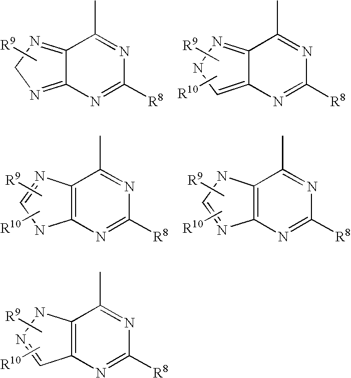
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl aryloxyalkyl, aralkoxyalkyl, alkylthio, thioalkyl groups, carboxylic acid or its derivatives, or sulfonic acid or its derivatives, which comprises, hydrolysing a compound of formula (I) described in any of the claims 5, 6 and 7, wherein R³ represents substituted or unsubstituted groups selected from alkyl, cycloalkyl, aryl, aralkyl, heterocyclyl, heteroaryl or heteroaralkyl groups and all other symbols are as defined above by conventional methods.

10. A process for the preparation of compound of formula (I) 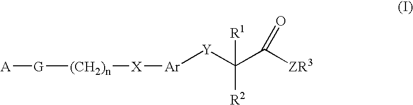
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 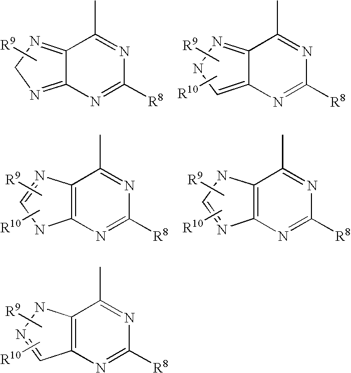
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives, which comprises:
a) reacting a compound of formula (I) where all symbols are as defined above and Y represent oxygen or ZR³ represents a halogen atom or COZR³ represents a mixed anhydride group with appropriate amines of the formula NHR³R⁴, where R³ and R³ are as defined above and if desired;
b) converting the compounds of formula (I) obtained above into pharmaceutically acceptable salts or pharmaceutically acceptable solvates by conventional methods.

11. An intermediate of formula (IIIa)
A-G-(CH₂)ₙ—X—Ar—CHO (IIIa)
their derivatives, their analogs, their tautomeric forms, their stereoisomers, their polymorphs, their pharmaceutically acceptable salts, their pharmaceutically acceptable solvates where Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; m and p are integers ranging from 0-4; n is an integer in the range of 1-4; A represents pyrazolopyrimidine or imidazolopyrimidine of the formula given below: 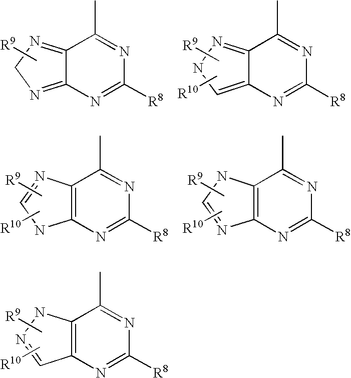
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives.

12. An intermediate of formula (IVb) 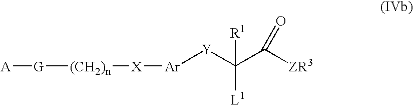
their derivatives, their analogs, their tautomeric forms, their stereoisomers, their polymorphs, their pharmaceutically acceptable salts, their pharmaceutically acceptable solvates where L² is a leaving group such as halogen atom, p-toluenesulfonate, methanesulfonate, trifluoromethanesulfonate and the like; R¹ represents hydrogen atom, halogen, hydroxy, alkyl, alkoxy, acyl, substituted or unsubstituted aralkyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, (CH₂)ₚO, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 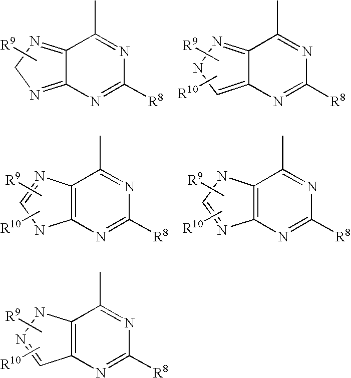
where R⁸ and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives.

13. An intermediate of formula (IVf) 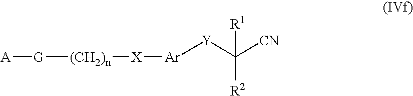
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; Ar represents substituted or unsubstituted, divalent, single or fused, aromatic, heteroaromatic, or heterocyclic group; G represents O or S; X represents O, NHR⁵, —CO(CH₂)ₚNR⁵(CH₂)ₘ—, (CH₂)ₚO, —(CH₂)NR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4; n is an integer in the range of 1-4; A represents pyrazolopyrimidine or imidazolopyrimidine of the formula given below: 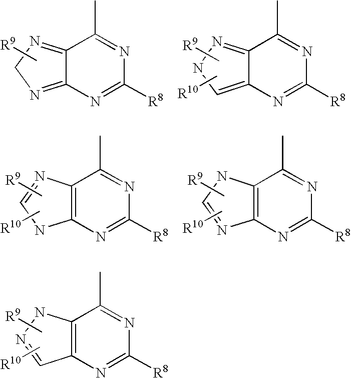
where and R⁹, R¹⁰ when attached to carbon atom may be same or different and represent hydrogen, halogen, hydroxy, nitro, cyano, formyl or substituted or unsubstituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl, cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives.

14. An intermediate of formula (IVg) 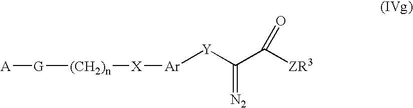
their derivatives, their analogs, their tautomeric forms, their stereoisomers, their polymorphs, their pharmaceutically acceptable salts, their pharmaceutically acceptable solvates where R³ represents-hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; G represents O or S; X represents O, NHR⁵, —CO(CH₂)ₚNR⁵(CH₂)ₘ—, (CH₂)ₚO, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl, heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group; m and p are integers ranging from 0-4; n is an integer in the range of 1-4; A represents pyrazolopyrimidine or imidazolopyrimidine of the formula given below: 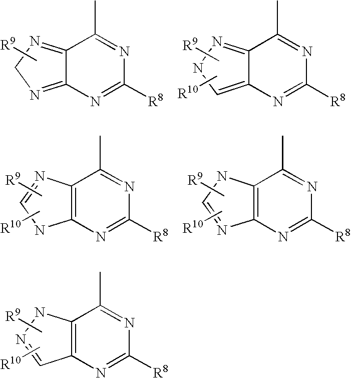
where R⁸ and R⁹, R₁₀ when attached to carbon atom may be same or different and represent hydrogen, halogen, hydroxy, nitro, cyano, formyl or substituted or unsubstituted groups selected from alkyl cycloalkyl, alkoxy, cycloalkoxy, aryl, aryloxy, aralkyl, aralkoxy, heterocyclyl, heteroaryl, heteroaralkyl, heteroaryloxy, heteroaralkoxy, alkanoyl, aroyl, alkanoyloxy, hydroxyalkyl, amino, alkanoylamino, monoalkylamino, dialkylamino, arylamino, aralkylamino, aminoalkyl alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R⁹ and R¹⁰ when attached to nitrogen atom represents hydrogen, hydroxy, formyl or substituted or unsubstituted groups selected from alkyl cycloalkyl, alkoxy, cycloalkoxy, aryl, aralkyl, aryloxy, aralkoxy, heterocyclyl, heteroaryl, heteroaralkyl, heteroaryloxy, heteroaralkoxy, alkanoyl, aroyl, alkanoyloxy, hydroxyalkyl, aminoalkyl, alkoxycarbonyl, aryloxycarbonyl, aralkoxycarbonyl, alkoxyalkyl, aryloxyalkyl, aralkoxyalkyl, alkylthio, thioalkyl groups, carboxylic acid or its derivatives, or sulfonic acid or its derivatives.

15. An intermediate of formula (IIIf) 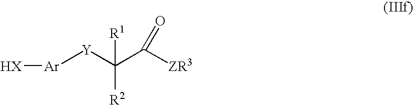
their derivatives, their analogs, their tautomeric forms, their stereoisomers, their polymorphs, their pharmaceutically acceptable salts, their pharmaceutically acceptable solvates where R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl carboxyalkyl, alkanoyl, aroyl, aralkanoyl heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl heteroaralkyl groups or (C₁-C₁₂)alkylcarboxylic acid and its derivatives; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; m and p are integers ranging from 0-4.

16. A process for preparing the compound of formula (IIIf) 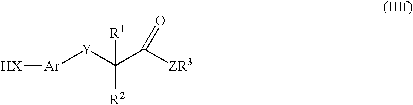
wherein all the symbols are as defined in claim 15, which comprises:
a. reducing a compound of formula (IIIf-1) 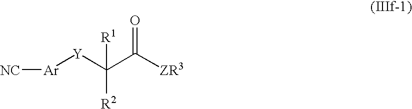
where all symbols are as defined in claim 15, in the presence of gaseous hydrogen and a catalyst selected from Pd/C, Rh/C, Pt/C or a mixture thereof. The reaction is carried out in the presence of a solvent selected from dioxane, acetic acid, ethyl acetate, alcohol like methanol or ethanol or a mixture thereof. A pressure between atmospheric pressure and 40 to 80 psi is employed. The catalyst used is preferably 5-10% Pd/C and the amount from 5-100% w/w. Alternatively the reaction can be carried out by employing metal solvent reduction selected from magnesium or samarium in alcohol or sodium amalgam in alcohol, preferably methanol to yield a compound of formula (IIIf) 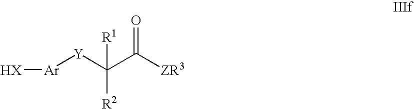
where all symbols are as defined earlier or
b. reacting a compound of formula (IIIf-2) 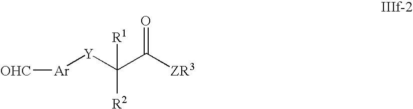
where all symbols are as defined earlier with a compound of formula (IIIf-3)
R⁵—NH₂ IIIf-3
where R⁵ represents alkyl group using a solvent selected from CH₂Cl₂, CHCl₃, chlorobenzene, benzene, THF, in the presence of catalyst selected from p-toluenesulfonic acid, methanesulfonic acid, TFA, TfOH, BF₃—OEt₂ or in the presence of activated molecular sieves at a temperature in the range of 10° C. to 100° C., preferably at a temperature in the range from 10° C. to 60° C. for a period in the range of 1 h to 48 h. The imine thus obtained is reduced in the presence of Na(CN)BH₃—HCl to obtain the compound of formula (IIIf) where R⁵ represents alkyl group and all other symbols are as defined earlier. Or
c. Conversion of the compound of formula (IIIf-4) 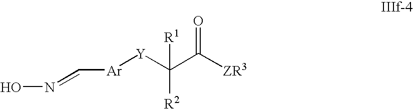
where R⁵ represents hydrogen and all other symbols as defined earlier, in the presence of a solvent selected from MeOH, EtOH or i-PrOH using hydroxylamine hydrochloride, in the presence or absence of a promoter selected from NaOAc, KOAc or a mixture thereof. The reaction is carried out at a temperature in the range of room temperature to the reflux temperature of the solvent used for a period in the range of 2 h to 24 h, preferably in the range 2 h to 12 h. The imine product thus obtained above may be reduced by using Na(CN)BH₃—HCl to obtain the compound of formula (IIIf) wher R⁵ represents hydrogen and all other symbols are as defined earlier. Or
d. reducing the compound of formula (IIIf-5) 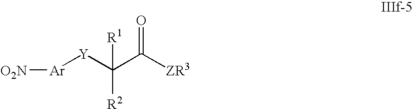
to a compound of formula (IIIf) where R⁵ represents hydrogen and all other symbols are as defined earlier, in the presence of gaseous hydrogen and a catalyst selected from Pd/C, Rh/C, Pt/C or a mixture thereof. The reaction is carried out in the presence of a solvent selected from dioxane, acetic acid, ethyl acetate, alcohol like methanol or ethanol or a mixture thereof. A pressure between atmospheric pressure and 40 to 80 psi is employed. The catalyst used is preferably 5-10% Pd/C and the amount from 5-100% w/w. Alternatively the reaction can be carried out by employing metal solvent reduction selected from magnesium or samarium in alcohol or sodium amalgam in alcohol, preferably methanol. Or
e. i. diazotization of the compound of formula (IIIf-6) 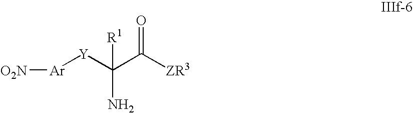
where Z is as defined earlier excluding NH and all other symbols are as defined earlier to obtain compound of formula (IIIf-7) 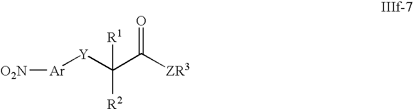
where R² represents substituted or unsubstituted groups selected from alkoxy, aryloxy, aralkoxy, heteroaryloxy, heteroaralkoxy and all other symbols are as defined earlier is carried out using a diazotizing agent selected from sodium nitrite, isoamyl nitrite, potassium nitrite or ammonium nitrite under aqueous acidic conditions using an acid selected from sulfuric acid, HCl or acetic acid, in an organic solvent selected from an alcohol like methanol, ethanol or propanol; 1,4-dioxane, THF or acetone. Etherifying the residue obtained using an alkylating agent selected from alkyl sulfate like diethyl sulphate or dimethylsulphate; alkyl halide like ethyl iodide or methyliodide, in a solvent selected from a hydrocarbon like toluene or benzene or DMF, DMSO or methyl isobutyl ketone (MIBK), in the presence of a alkali base selected from sodium carbonate, potassium carbonate, sodium methoxide, sodium hydride or potassium hydride.
ii. The compound of formula (IIIf-7) is converted to a compound of formula (IIIf) where R⁵ represents hydrogen atom and all other symbols are as defined earlier in the presence of gaseous hydrogen and a catalyst selected from Pd/C, Rh/C, Pt/C or a mixture thereof. The reaction is carried out in the presence of a solvent selected from dioxane, acetic acid, ethyl acetate, alcohol like methanol or ethanol or a mixture thereof. A pressure between atmospheric pressure and 40 to 80 psi is employed. The catalyst used is preferably 5-10% Pd/C and the amount from 5-100% w/w. Alternatively the reaction can be carried out by employing metal solvent reduction selected from magnesium or samarium in alcohol or sodium amalgam in alcohol, preferably methanol. Or
f. i. reacting a compound of formula (IIIb) 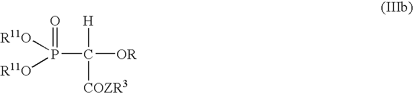
where all symbols are as defined earlier, with a compound of formula (IIIf-9) 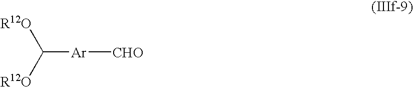
where R¹² represents (C₁-C₆)alkyl group to yield compound of formula (IIIf-10) 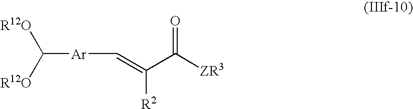
where Z is as defined earlier excluding NH and all symbols are as defined earlier, is carried out in the presence of a base like a metal hydride like NaH or KH; organolithiums like CH₃Li or BuLi; alkoxides like NaOMe, NaOEt or t-BuO⁻K⁺ or a mixture thereof. The reaction is carried out in the presence of a solvent selected from diethyl ether, THF, dioxane, DMF, DMSO, DME, dimethyl acetamide or a mixture thereof in the presence or absence of HMPA as a cosolvent at a temperature in the range of −78° C. to 50° C., preferably at a temperature in the range of −10° C. to 30° C.
ii. The compound of formula (IIIf-10) is reduce to compound of formula (IIIf-2) 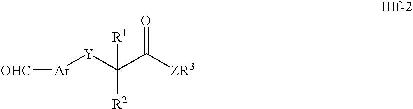
where all symbols are as defined earlier in the presence of gaseous hydrogen and a catalyst selected from Pd/C, Rh/C, Pt/C or a mixture thereof. The reaction is carried out in the presence of a solvent selected from dioxane, acetic acid, ethyl acetate, alcohol like methanol or ethanol or a mixture thereof. A pressure between atmospheric pressure and to 80 psi is employed. The catalyst used is preferably 5-10% Pd/C and the amount from 5-50% w/w. Alternatively the reaction can be carried out by employing metal solvent reduction selected from magnesium, iron, tin or samarium in alcohol or sodium amalgam in alcohol, preferably methanol.
iii. reacting a compound of formula (IIIf-2)
R⁵—NH₂ IIIf-3
where R⁵ is as defined earlier using a solvent selected from CH₂Cl₂, CHCl₃, chlorobenzene, benzene, THF, in the presence of catalyst selected from p-toluenesulfonic acid, methanesulfonic acid, TFA, TfOH, BF₃—OEt₂ or in the presence of activated molecular sieves at a temperature in the range of 10° C. to 100° C., preferably at a temperature in the range from 10° C. to 60° C. for a period in the range of 1 h to 48 h. The imine thus obtained is reduced in the presence of Na(CN)BH₃—HCl to obtain the compound of formula (IIIf) where R⁵ represents alkyl group and all other symbols are as defined earlier.

17. An intermediate of the formula (IIId) 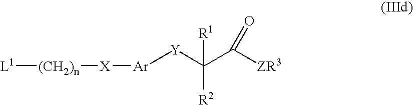
their derivatives, their analogs, their tautomeric forms, their stereoisomers, their polymorphs, their pharmaceutically acceptable salts, their pharmaceutically acceptable solvates where L¹ is a leaving group such as halogen atom, p-toluenesulfonate, methanesulfonate, trifluoromethanesulfonate; R¹ represents hydrogen atom, halogen, hydroxy, alkyl, alkoxy, acyl, substituted or unsubstituted aralkyl groups; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; Y represents O, S. NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; n is an integer in the range of 1-4; m and p are integers ranging from 0-4.

18. A process for preparation of the compound of formula (IIId) 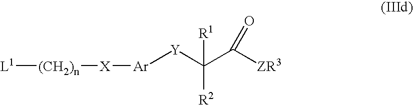
where all the symbols are as defined in claim 17, which comprises reacting the compound of formula (IIIf) 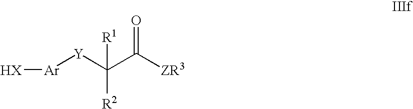
where X represents NHR⁵ and all other symbols are as defined earlier with compound of formula (IIId-1)
L¹-(CH₂)ₙ—CO-L² (IIId-1)
where L¹ and n are as defined earlier and L² represents halogen, in the presence of a solvent selected from DCM, DCE, THF, DMF, DMSO, DME or a mixture thereof. The reaction is carried out in an inert atmosphere maintained by using inert a gase like N₂, Ar or He, in the presence of a base selected from triethyl amine, lutidine, collidine or a mixture thereof at a temperature in the range of −20° C.-120° C. for a period in the range oft to 48 hours, preferably from 2 to 12 hours to yield a compound of formula (IIId) where all symbols are as defined earlier.

19. An intermediate of the formula (IIId) 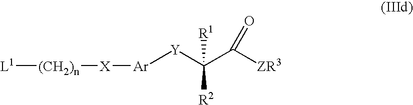
their derivatives, their analogs, their tautomeric forms, their polymorphs, their pharmaceutically acceptable salts, their pharmaceutically acceptable solvates where L¹ is a leaving group such as halogen, p-toluenesulfonate, methanesulfonate, trifluoromethanesulfonate; R¹ represents hydrogen; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl, aryl, aralkyl, heterocyclyl, heteroaryl or heteroaralkyl groups; Z represents oxygen or NR⁴, where R⁴ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; n is an integer in the range of 1-4; m and p are integers ranging from 0-4.

20. A process for preparation of the compound of formula (IIId) 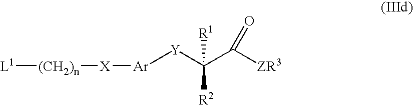
where all the symbols are as defined in claim 19, which comprises reacting the compound of formula (IIIf) 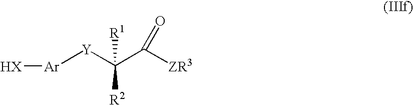
where X represents NHR⁵ and all other symbols are as defined above with compound of formula (IIId-1)
L¹-(CH₂)ₙ—CO-L² (IIId-1)
where L¹ and n are as defined earlier and L² represents halogen, in the presence of a solvent selected from DCM, DCE, THF, DMF, DMSO, DME or a mixture thereof. The reaction is carried out in an inert atmosphere maintained by using inert a gase like N₂, Ar or He, in the presence of a base selected from triethyl amine, lutidine, collidine or a mixture thereof at a temperature in the range of −20° C.-120° C. for a period in the range of 1 to 48 hours, preferably from 2 to 12 hours to yield a compound of formula (IIId) where all symbols are as defined earlier.

21. An intermediate of the formula (IIId) 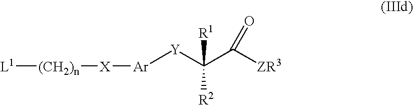
their derivatives, their analogs, their tautomeric forms, their polymorphs, their pharmaceutically acceptable salts, their pharmaceutically acceptable solvates where L¹ is a leaving group such as halogen, p-toluenesulfonate, methanesulfonate, trifluoromethanesulfonate; R¹ represents hydrogen; R² represents hydrogen, hydroxy, halogen, substituted or unsubstituted groups selected from alkyl, cycloalkyl, cycloalkylalkyl, alkoxy, aryl, alkanoyl, alkanoyloxy, aroyl, aralkyl, aryloxy, aralkoxy, heterocyclyl, heteroaryl, heteroaralkyl, heteroaryloxy, heteroaralkoxy, alkoxyalkyl, alkoxycarbonyl, aryloxycarbonyl, alkylaminocarbonyl, arylaminocarbonyl groups; R³ represents hydrogen or substituted or unsubstituted groups selected from alkyl, cycloalkyl aryl, aralkyl, heterocyclyl, heteroaryl or heteroaralkyl groups; Z represents oxygen or NR⁴, where R⁴ represents hydrogen or substituted or unsubstituted groups selected from alkyl aryl, aralkyl, hydroxyalkyl, carboxyalkyl, alkanoyl, aroyl, aralkanoyl, heterocyclyl, heteroaryl or heteroaralkyl groups or R³ and R⁴ together may form a substituted or unsubstituted 5 or 6 membered cyclic structure containing carbon atoms, a nitrogen atom and which may optionally contain one or two additional heteroatoms selected from oxygen, sulfur or nitrogen; Ar represents substituted or unsubstituted, divalent, single or fused, aromatic, heteroaromatic or heterocyclic group; X represents O, NHR⁵, —CO(CH₂)ₚNR⁵(CH₂)ₘ—, —(CH₂)ₚO—, —(CH₂)ₚNR⁵CO—; where R⁵ represents hydrogen or substituted or unsubstituted groups selected from alkyl aryl, aralkyl, hydroxyalkyl, carboxyalkyl, alkanoyl, aroyl, aralkanoyl, heterocyclyl, heteroaryl or heteroaralkyl groups; Y represents O, S, NR⁶ or CHR⁷; where R⁶ represents hydrogen or substituted or unsubstituted groups selected from alkyl, aryl, aralkyl, hydroxyalkyl, carboxyalkyl, alkanoyl, aroyl, aralkanoyl, heterocyclyl, heteroaryl or heteroaralkyl groups; R⁷ represents hydrogen atom, halogen, hydroxy, alkyl, alkoxy, substituted or unsubstituted aralkyl group or forms a bond together with the adjacent group R¹; n is an integer in the range of 1-4; m and p are integers ranging from 0-4.

22. A process for preparation of the compound of formula (IIId) 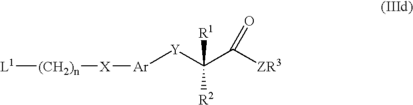
where all the symbols are as defined in claim 19, which comprises reacting the compound of formula (IIIf) 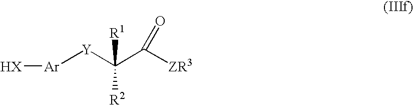
where X represents NHR⁵ and all other symbols are as defined above with compound of formula (IIId-1)
L¹-(CH₂)ₙ—CO-L² (IIId-1)
where L¹ and n are as defined earlier and L² represents halogen, in the presence of a solvent selected from DCM, DCE, THF, DMF, DMSO, DME or a mixture thereof. The reaction is carried out in an inert atmosphere maintained by using inert a gase like N₂, Ar or He, in the presence of a base selected from triethyl amine, lutidine, collidine or a mixture thereof at a temperature in the range of −20° C.-120° C. for a period in the range of 1 to 48 hours, preferably from 2 to 12 hours to yield a compound of formula (IIId) where all symbols are as defined earlier.

23. A compound according to claim 1, wherein when the groups represented by R⁵ are substituted, the substituents are selected from halogen, hydroxy, nitro or substituted or unsubstituted groups selected from alkyl, cycloalkyl, alkoxy, cycloalkoxy, aryl, aralkyl, aryloxy, aralkoxy, aralkoxyalkyl, heterocyclyl, heteroaryl, heteroaralkyl, hydroxyalkyl, amino, arylamino, aminoalkyl, alkylamino, alkoxyalkyl, alkylthio, thioalkyl groups, carboxylic acid or its derivatives; sulfonic acid or its derivatives.

24. A pharmaceutical composition which comprises a compound of formula (I) 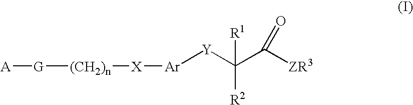
as defined in claim 1 and a pharmaceutically acceptable carrier, diluent, excipient or solvate.

25. A pharmaceutical composition as claimed in claim 24, in the form of a tablet, capsule, powder, syrup, solution or suspension.

26. A composition which comprises a compound of formula (I) as defined in claim 1 or a compound as claimed in claim 4 and an HMG CoA reductase inhibitor; cholesterol absorption inhibitor; antiobesity drug; lipoprotein disorder treatment drug; hypoglycemic agent; insulin; biguanide; sulfonylurea; thiazolidinedione; dual PPARα and γ agonis or a mixture thereof.

27. A method of preventing or treating hyperlipemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a compound of formula (I) as defined in claim 1 or a compound as claimed in claim 4 or a pharmaceutical composition according to claim 24 or 25 to a patient in need thereof.

28. A method according to claim 27, wherein the disease is type II diabetes, impaired glucose tolerance, dyslipidemia, disorders related to Syndrome X including hypertension, obesity, insulin resistance, coronary artery disease and other cardiovascular disorders; renal diseases including glomerulonephritis, glomerulosclerosis, nephrotic syndrome, hypertensive nephrosclerosis, retinopathy, nephropathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer or osteoporosis or as inflammatory agents.

29. A method according to claim 27, for the treatment and/or prophylaxis of disorders related to Syndrome X, which comprises administering an agonist of PPARα and/or PPARγ of formula (I) as claimed in claim 1 or a compound as claimed in claim 4 or a pharmaceutical composition according to claim 24 or 25 to a patient in need thereof.

30. A method of reducing total cholesterol, body weight, blood plasma glucose, triglycerides, LDL, VLDL or free fatty acids or increasing HDL in the plasma comprising administering a compound of formula (I), as defined in claim 1 or a compound as claimed in claim 4 or a pharmaceutical composition according to claim 24 or 25 to a patient in need thereof.

31. A method of preventing or treating hyperlipemia, hypercholesteremia, hyperglycemia, osteoporosis, obesity, impaired glucose tolerance, atherosclerosis, leptin resistance, insulin resistance, or diseases in which insulin resistance is the underlying pathophysiological mechanism comprising administering to a patient in need thereof an effective amount of a compound of formula (I) as defined in claim 1 or a compound as claimed in claim 4 or a pharmaceutical composition according to claim 24 or 25 in combination/concomittant with a HMG CoA reductase inhibitor; cholesterol absorption inhibitor; antiobesity drug; lipoprotein disorder treatment drug; hypoglycemic agent; insulin; biguanide; sulfonylurea; thiazolidinedione; dual PPARα and γ agonis or a mixture thereof within such a period so as to act synergistically.

32. A method according to claim 31, wherein the disease is type II diabetes, impaired glucose tolerance, dyslipidemia, disorders related to Syndrome X such as hypertension, obesity, insulin resistance, coronary artery disease and other cardiovascular disorders; certain renal diseases including glomerulonephritis, glomerulosclerosis, nephrotic syndrome, hypertensive nephrosclerosis, retinopathy, nephropathy, disorders related to endothelial cell activation, psoriasis, polycystic ovarian syndrome (PCOS), dementia, diabetic complications, osteoporosis, inflammatory bowel diseases, myotonic dystrophy, pancreatitis, arteriosclerosis, xanthoma, eating disorders, cancer or as inflammatory agents.

33. A method according to claim 31, for the treatment and/or prophylaxis of disorders related to Syndrome X which comprises administering to a patient in need thereof an agonist of PPARα and/or PPARγ of formula (I) as claimed in claim 1 or a compound as claimed in claim 4 or a pharmaceutical composition according to claim 24 or 25 and a HMG CoA reductase inhibitor; cholesterol absorption inhibitor; antiobesity drug; lipoprotein disorder treatment drug; hypoglycemic agent; insulin; biguanide; sulfonylurea; thiazolidinedione; dual PPARα and γ agonis or a mixture thereof within such a period as to act synergistically.

34. A method of reducing plasma glucose, triglycerides, total cholesterol, LDL, VLDL or free fatty acids or increasing HDL in the plasma, which comprises administering a compound of formula (I) claimed in claim 1 or a compound as claimed in claim 4 or a pharmaceutical composition according to claim 24 or 25, in combination/concomittant with a HMG CoA reductase inhibitor; cholesterol absorption inhibitor; antiobesity drug; lipoprotein disorder treatment drug; hypoglycemic agent; insulin; biguanide; sulfonylurea; thiazolidinedione; dual PPARα and γ agonis or a mixture thereof which may be administered together or within such a period as to act synergistically together to a patient in need thereof.
